(19) United States
(12) Patent Application Publication
(10) Pub. No.: US 2007/0218461 A1
(43) Pub. Date: Sep. 20, 2007

(54) Indole-Diterpene Biosynthesis

(76) Inventors: Gregory Thomas Bryan, Feilding (NZ); Richard Johnson, Palmerston North (NZ); Barry Scott, Palmerston North (NZ); Carolyn A. Young, Ardmore, OK (US); Brian Anthony Tapper, Palmerston North (NZ); Emily Jane Parker, Palmerston North (NZ)

Correspondence Address: KNOBBE MARTENS OLSON & BEAR LLP, 2040 MAIN STREET, FOURTEENTH FLOOR, IRVINE, CA, 92614, US

(21) Appl. No.: 10/584,429

(22) PCT Filed: Dec. 22, 2004
(86) PCT No.: PCT/NZ04/00333

§ 371 Date: Oct. 27, 2006

(30) Foreign Application Priority Data
Dec. 22, 2003 (NZ) 530331

(51) Int. Cl. C12Q 1/68; A01H 17/00; C07H 21/04; C07K 14/37; C12N 1/21; C12N 15/01; C12N 15/63; C12N 9/00

(52) U.S. Cl. 435/6; 435/117; 435/183; 435/252.3; 435/440; 530/371; 536/23.1; 536/24.32; 536/24.33; 800/295

(57) ABSTRACT

The present invention relates to the biosynthesis of indole diterpene compounds. In particular, the invention relates to genes encoding enzymes considered responsible for the synthesis of lolitrems.

### TECHNICAL FIELD

The present invention relates to the biosynthesis of indole diterpene compounds. In particular, the invention relates to genes encoding enzymes considered responsible for the synthesis of lolitrems.

### BACKGROUND ART

Indole-Diterpenes

The indole-diterpenes are a large, structurally diverse group of natural products principally found in filamentous fungi notably of the genera Penicillium, Aspergillus, Claviceps, Epichloe and Neotyphodium (Steyn and Vleggaar 1985; Mantle 1987; Scott et al. 2003). They may be classified into the following structural sub-groups, the penitrems, janthitrems, sulphinines (Laakso et al., 1992), nodulisporic acid (Ondeyka et al., 1997) and thiersinines (Li et al., 2002), and lolitrems. These metabolites all have a common core structure comprised of a cyclic diterpene skeleton derived from geranylgeranyl diphosphate (GGPP) and an indole moiety derived from either tryptophan or a tryptophan precursor (Acklin et al. 1977; de Jesus et al. 1983; Laws and Mantle 1989). Further complexity of the carbon skeleton is achieved by additional prenylations, different patterns of ring substitutions and different ring stereochemistry. Many of these compounds are potent mammalian tremorgens (Cole and Cox 1981) while others are known to have confer anti-insect activity (Gloer 1995).

Paxilline Biosynthesis

Until recently, very little was known about the pathways for the biosynthesis of the indole-diterpenes, although putative biosynthetic schemes have been proposed on the basis of chemical identification of likely intermediates from the organism of interest and related filamentous fungi (Mantle and Weedon 1994; Munday-Finch et al. 1996; Gatenby et al. 1999). The recent cloning and characterization of a cluster of genes from Penicillium paxilli required for the biosynthesis of paxilline has provided for the first time an insight into the genetics and biochemistry of indole-diterpene biosynthesis (Young et al. 2001).

Key genes identified in this cluster include a GGPP synthase (paxG), a FAD-dependent monooxygenase (paxM), a prenyl transferase (paxC) and two cytochrome P450 monooxygenases, paxP and paxQ. Deletion of paxG resulted in mutants that were paxilline negative, confirming that this gene is essential for paxilline biosynthesis (Young et al. 2001). Targeted deletion of paxM and paxC in P. paxilli also result in mutants that are defective in paxilline biosynthesis (B. Scott, L. McMillan, J. Astin, C. Young, E. Parker, unpublished results). It is proposed that PaxM and paxC are required to catalyse the addition of indole-3-glycerol phosphate to GGPP and subsequent cyclisation to form the first stable indole-diterpene, possibly paspaline (Parker and Scott 2004). Deletion of paxP and paxQ give rise to strains that accumulate paspaline and 13-desoxypaxilline, respectively, suggesting that these are the substrates for the corresponding enzymes (McMillan et al. 2003). Overall, these results establish that at least 5 genes are required for the biosynthesis of paxilline in P. paxilli.

The identification of a geranyl-geranyl diphosphate (GGPP) synthase gene (paxG) within this cluster, and confirmation by deletion analysis that it is necessary for paxilline biosynthesis, suggest that the synthesis of GGPP is one of the first steps in the synthesis of this indole-diterpene (Young et al. 2001). P. paxilli, like Gibberella fujikuroi (Mende et al. 1997: Tudzynski and Hölter 1998), recently renamed Fusarium fujikuroi (O'Donnell et al. 1998), has two GGPP synthase genes, but the second, ggsl, is unable to complement the paxG deletion, presumably because of cellular partitioning of the two enzymes (Young et al. 2001). The synthesis of paxilline is predicted to involve several oxygenation steps (Munday-Finch et al. 1996), and the presence within the cluster of genes for two FAD-dependent monooxygenases (paxM and paxn) and for two cytochrome P450 monooxygenases (paxP and paxQ) is consistent with this chemistry (Young et al. 2001).

The only other fungal diterpene gene cluster reported to date is that for the biosynthesis of gibberellins in Fusarium fujikuroi (teleomorph Gibberella fujikuroi) (Tudzynski and Hölter 1998). This cluster also includes a GGPP synthase gene, ggs-2, required for the first committed step in gibberellin biosynthesis. Interestingly, both fungal species contain an additional copy of a GGPP synthase gene, ggs1 in P. paxilli (Young et al. 2001) and ggs-1 in F. fujikuroi (Mende et al. 1997). This suggests that the presence of two copies of GGPP synthases could be a molecular signature for diterpene biosynthesis in filamentous fungi, one copy being required for primary metabolism and the second for secondary (diterpene) metabolism. Given that genes for secondary metabolite biosynthesis in fungi are generally organised in clusters (Keller and Hohn 1997), molecular cloning of GGPP synthases combined with chromosome walking provides a rapid strategy for cloning new indole-diterpene gene clusters.

Lolitrems

Epichloë/Neotyphodium endophytes are a group of Clavicipitaceous fungi (Clavicipitaceae, Ascomycota) that form symbiotic associations with temperate grasses such as perennial ryegrass and tall fescue (Schardl 2001; Scott 2001). The plant provides nutrients for the endophyte and a means of dissemination through the seed. The endophyte protects the host from biotic (e.g. insect and mammalian herbivory) and abiotic stress (e.g. drought). Fungal synthesis of secondary metabolites appears to be the main mechanism for protection of the symbiotum from herbivory.

The ability of Epichloë/Neotyphodium endophytes to synthesize bioprotective metabolites in planta constitutes a major ecological benefit for the symbiotum (Schardl 1996). Metabolites identified to date include both anti-insect (e.g. peramine and lolines) and anti-mammalian (ergot alkaloids and indole-diterpenes) (Bush et al. 1997). However, from an agricultural perspective endophyte production of mammalian toxins such as the indole-diterpenes of the lolitrem group with the moiety: 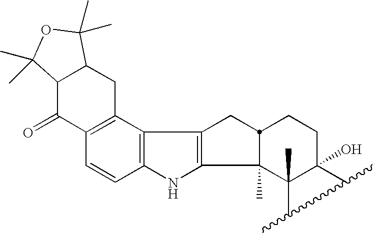
or derivatives thereof, and in particular lolitrem B: 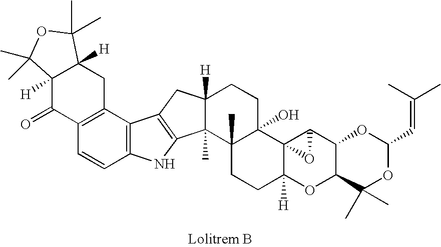
are detrimental to grazing livestock. Consequently, there is considerable commercial interest in developing associations containing endophytes that are not toxic to mammals (Fletcher 1999; Popay et al. 1999).

The lolitrems are produced by the Epichloë endophytes in association with temperate grass species (Gallagher et al., 1984). These fungi are often found as an infection in perennial ryegrass (Lolium perenne) and tall fescue grasses (Festuca arundinacea).

Endophytes are symbiotic fungi and are prevalent in New Zealand pastures. The fungal metabolites from these endophytes are thought to serve as chemical defence systems for the fungi that produce them. They may also be of use in protecting the food source from consumption by other organisms (U.S. Pat. No. 4,973,601).

However some of these fungi also pose a problem in that, at least lolitrem B, is known to be the main causative agent in ryegrass staggers (Fletcher and Harvey, 1981). This is a condition in which animals grazing on endophyte infected pastures develop ataxia, tremors, and hypersensitivity to external stimuli. The lolitrem neurotoxin (staggers) reaction is long acting but is however completely reversible (Smith et al 1997, McLeay et al 1999). The time course of tremors induced by lolitrem B is dramatically different from that of other indole diterpenes, for example paxilline and analogues. Paxilline analogues induce tremors of rapid onset and short duration while tremors induced by lolitrem derivatives take hours to reach maximum intensity and last for days.

The mechanism by which lolitrem B and related indole-diterpenes cause tremorgenicity in mammals is not well defined but biochemical and clinical studies indicate that these effects are due in part to effects on receptors and interference with neurotransmitter release in the central and peripheral nervous system (Selala et al. 1991). Some have been shown to potentiate chloride currents through GABA_{A} receptor chloride channels heterologously expressed in Xenopus oocytes (Yao et al. 1989). Many of the indole-diterpenes are potent inhibitors of high conductance Ca²⁺-activated K⁺ (maxi-K) channels (Knaus et al. 1994; McMillan et al. 2003)

It would therefore be useful if the genes involved in the biosysthesis of indole diterpenes related to lolitrems could be identified as this would provide information useful in: manipulating this biosynthetic pathway; producing indole diterpenes related to lolitrems; identifying mutations in endophytes which produce indole diterpenes related to lolitrems.

All references, including any patents or patent applications cited in this specification are hereby incorporated by reference. No admission is made that any reference constitutes prior art. The discussion of the references states what their authors assert, and the applicants reserve the right to challenge the accuracy and pertinency of the cited documents. It will be clearly understood that, although a number of prior art publications are referred to herein, this reference does not constitute an admission that any of these documents form part of the common general knowledge in the art, in New Zealand or in any other country.

It is acknowledged that the term ‘comprise’ may, under varying jurisdictions, be attributed with either an exclusive or an inclusive meaning. For the purpose of this specification, and unless otherwise noted, the term ‘comprise’ shall have an inclusive meaning—i.e. that it will be taken to mean an inclusion of not only the listed components it directly references, but also other non-specified components or elements. This rationale will also be used when the term ‘comprised’ or ‘comprising’ is used in relation to one or more steps in a method or process.

It is an object of the present invention to address the foregoing problems or at least to provide the public with a useful choice.

Further aspects and advantages of the present invention will become apparent from the ensuing description which is given by way of example only.

### DISCLOSURE OF INVENTION

According to one aspect of the present invention there is provided an isolated nucleic acid molecule having a nucleic acid sequence selected from the group consisting of:
a) SEQ ID NOs 1, 3, 5, 17, 19, 21, 7, 9, 11, 13 and 15 or a combination of these sequences;
b) SEQ ID NOs 23, 24 and 25;
c) a functional fragment or variant of the sequences in a) or b);
d) a complement to the sequences in a), b) or c).

In some embodiments the isolated nucleic acid molecule may have at least 70% sequence homology to a nucleic acid molecule substantially as described above.

More preferably the isolated nucleic acid molecule may have:
at least 80% sequence homology or
at least 90% sequence homology or
at least 95% sequence homology to a nucleic acid substantially as described above.

Most preferably the isolated nucleic acid molecule may have at least 99% sequence homology to a nucleic acid molecule substantially as described above.

According to another aspect of the present invention there is provided an isolated polypeptide having an amino acid sequence selected from the group consisting of:
a) SEQ ID NOs 2, 4, 6, 8, 10, 12, 14, 16, 18, 20, and 22 or a combination of these sequences;
b) A functional fragment or variant of the sequences listed in a).

In some embodiments the isolated polypeptide molecule may have at least 70% sequence homology to a polypeptide substantially as described above.

More preferably the isolated nucleic acid molecule may have:
at least 70% sequence homology to a polypeptide or
at least 80% sequence homology to a polypeptide or
at least 90% sequence homology to a polypeptide or
at least 95% sequence homology to a polypeptide substantially as described above.

Most preferably the isolated polypeptide molecule may have at least 99% sequence homology to a polypeptide substantially as described above.

According to another aspect of the present invention there is provided a primer capable of binding to a nucleic acid molecule substantially as described above.

Most preferably a primer having a nucleotide sequence which comprises at least substantially 15-20 contiguous nucleotides of a nucleic acid molecule selected from the group consisting of: SEQ ID NOs. 1, 3, 5, 17, 19, 21, 7, 9, 11, 13 and 15.

In some preferred embodiments there may be a primer having a nucleotide sequence selected from the group consisting of SEQ ID NOs 26-51.

According to another aspect of the present invention there is a probe capable of binding to a nucleic acid molecule substantially as described above.

The use of a probe capable of binding to a nucleic acid molecule substantially as described above to identify at least one gene of the lolitrem gene cluster in an endophyte.

The said endophyte may preferably be derived from the Epichloë or Neotyphodium genus.

An isolated nucleic acid molecule which is able to stringently hybridize to a nucleic acid molecule substantially as described above.

An isolated nucleic acid molecule substantially as described above wherein the molecule is a primer.

An isolated nucleic acid molecule substantially as described above wherein the molecule is a probe.

A method for identifying mutations in the lolitrem gene cluster of an endophyte exhibiting useful phenotypic traits, characterized by the steps of:
a) identifying at least one gene in the lolitrem gene cluster of an endophyte;
b) sequencing the gene(s) identified at a);
c) comparing the sequence at b) to SEQ ID NOs 1, 3, 5, 17, 19, 21, 7, 9, 11, 13 and 15 or a combination of these sequences to ascertain any differences in nucleotide sequence.

Preferably, these phenotypic traits may include non-tremorgenic strains/isolates or strains with increased insecticidal activity including those that produce lolitrem intermediates and/or shearinines and/or janthitrems and/or which produce an effect or effects selected from: a less toxic effect, a more toxic effect, a desired agricultural effect, a desired biochemical effect, a desired neurological effect, a desired insecticidal effect, and combinations thereof.

An endophyte in which at least one of the genes in the lolitrem gene cluster has been mutated or otherwise disrupted to manipulate the indole diterpene biosynthetic pathway.

Preferably these include but are not limited to: lolitrem intermediates and/or shearinines, and/or janthitrems.

Preferably, indole diterpenes are lolitrem compounds.

The use of a nucleic acid molecule substantially as described above to produce an indole diterpene, enzyme, intermediate or other chemical compound associated with the indole diterpene biosynthetic pathway.

The use of a nucleic acid molecule substantially as described above to study the indole diterpene pathway.

A construct which includes a nucleic acid molecule substantially as described above.

A host cell which includes a non-endogenous nucleic acid molecule substantially as described above.

An endophyte which includes a non-endogenous nucleic acid molecule substantially as described above.

The use of a polypeptide substantially as described above to catalyze in vitro or in vivo a reaction involved in the biosynthesis of an idole diterpene.

A kit for identifying the lolitrem gene cluster which includes a probe.

A kit for identifying the lolitrem gene cluster which includes at least one primer pair.

A method of manipulating the indole diterpene biosynthetic pathway characterized by the step of altering a nucleic acid substantially as described above to produce a gene encoding a non-functional polypeptide.

The use of a gene produced by the method substantially as described above to manipulate the indole diterpene biosynthetic pathway.

An expression system which includes a non-endogenous nucleic acid molecule substantially as described above.

The use of an expression system substantially as described above to produce indole diterpene, enzyme, intermediate or other chemical compound associated with the indole diterpene biosynthetic pathway.

The use of a primer substantially as described above to amplify a nucleic acid molecule.

A plant including a cell which includes a non-endogenous nucleic acid molecule substantially as described above.

A plant substantially as described above wherein the plant is a grass.

A plant substantially as described above wherein the plant is a rye grass.

A plant substantially as described above wherein the cell is present as an endophyte.

The use of an isolated nucleic acid molecule in the biosysthesis of an indole diterpene.

Throughout this specification the terms ‘pax’ and ‘ltm’ refer to orthologous genes, i.e. genes present in two different species which are different to one another but to a certain extent correspond (having homology) as they were derived from a common ancestor. The prefix used relates to the compound expressed by the gene, i.e. paxiline in the case of pax and lolitrem in the case of ltm.

Further, for the purposes of the specification, the terms ‘biosynthesis’ or ‘biosynthetic’ refer to the production of a chemical compound in a living organism via processes of that organism.

The alteration of a nucleic acid molecule to produce a gene expressing a non-functional polypeptide may be achieved in a variety of different ways which may include mutagenesis or gene silencing using techniques well known in the art.

The term ‘manipulate’ or ‘manipulating’ as used herein refers to the ability to up-regulate or down-regulate or otherwise control the indole diterpene biosynthetic pathway.

The term ‘non-endogenous nucleic acid’ as used herein refers to a nucleic acid molecule that does not naturally occur within a organism.

The term ‘expression system’ refers to any cell which can be used to express the polypeptides encoded by at least one nucleic acid molecule of interest. In general suitable cells for use as expression systems include bacteria, yeast, fungi, plants and animal cells.

The term ‘indole diterpene’ refers to any compound having a cyclic diterpene skelton derived from geranylgeranyl diphosphate (GGPP) and an indole moiety derived from either tryptophan or a tryptophan precursor. Most preferably the term indole diterpene refers to a lolitrem compound.

The term ‘non-functional’ refers to a polypeptide which is incapable of acting as an enzyme in indole diterpene biosynthesis.

It is to be clearly understood that the invention also encompasses peptide analogues, which include but are not limited to the following:
1. Compounds in which one or more amino acids is replaced by its corresponding D-amino acid. The skilled person will be aware that retro-inverso amino acid sequences can be synthesised by standard methods; see for example Choreo and Goodman, 1993;
2. Peptidomimetic compounds, in which the peptide bond is replaced by a structure more resistant to metabolic degradation. See for example Olson et al, 1993; and
3. Compounds in which individual amino acids are replaced by analogous structures for example, gem-diaminoalkyl groups or alkylmalonyl groups, with or without modified termini or alkyl, acyl or amine substitutions to modify their charge.

The use of such alternative structures can provide significantly longer half-life in the body, since they are more resistant to breakdown under physiological conditions.

Methods for combinatorial synthesis of peptide analogues and for screening of peptides and peptide analogues are well known in the art (see for example Gallop et al, 1994; Hogan, 1997).

For the purposes of this specification, the term “peptide and peptide analogue” includes compounds made up of units which have an amino and carboxy terminus separated in a 1,2, 1,3, 1,4 or larger substitution pattern. This includes the 20 naturally-occurring or “common” α-amino acids, in either the L or D configuration, the biosynthetically-available or “uncommon” amino acids not usually found in proteins, such as 4-hydroxyproline, 5-hydroxylysine, citrulline and ornithine; synthetically-derived α-amino acids, such as α-methylalanine, norleucine, norvaline, Cα- and N-alkylated amino acids, homocysteine, and homoserine; and many others as known in the art.

It also includes compounds that have an amine and carboxyl functional group separated in a 1,3 or larger substitution pattern, such as β-alanine, γ-amino butyric acid, Freidinger lactam (Freidinger et al, 1982), the bicyclic dipeptide (BTD) (Freidinger et al, 1982; Nagai and Sato, 1985), amino-methyl benzoic acid (Smythe and von Itzstein, 1994), and others well known in the art. Statine-like isosteres, hydroxyethylene isosteres, reduced amide bond isosteres, thioamide isosteres, urea isosteres, carbamate isosteres, thioether isosteres, vinyl isosteres and other amide bond isosteres known to the art are also useful for the purposes of the invention.

A “common” amino acid is a L-amino acid selected from the group consisting of glycine, leucine, isoleucine, valine, alanine, phenylalanine, tyrosine, tryptophan, aspartate, asparagine, glutamate, glutamine, cysteine, methionine, arginine, lysine, proline, serine, threonine and histidine. These are referred to herein by their conventional three-letter or one-letter abbreviations.

An “uncommon” amino acid includes, but is not restricted to, one selected from the group consisting of D-amino acids, homo-amino acids, N-alkyl amino acids, dehydroamino acids, aromatic amino acids (other than phenylalanine, tyrosine and tryptophan), ortho-, meta- or para-aminobenzoic acid, ornithine, citrulline, norleucine, α-glutamic acid, aminobutyric acid (Abu), and α-α disubstituted amino acids.

The term “nucleic acid molecule” as used herein may be an RNA, cRNA, genomic DNA or cDNA molecule, and may be single- or doublestranded. The nucleic acid molecule may also optionally comprise one or more synthetic, non-natural or altered nucleotide bases, or combinations thereof.

“Primers” are short nucleic acids, preferably DNA oligonucleotides 15 nucleotides or more in length, which are annealed to a complementary target DNA strand by nucleic acid hybridization to form a hybrid between the primer and the target DNA strand, then extended along the target DNA strand by a polymerase, preferably a DNA polymerase. Primer pairs can be used for amplification of a nucleic acid sequence, e.g. by the polymerase chain reaction (PCR) or other nucleic acid amplification methods well known in the art. PCR-primer pairs can be derived from the sequence of a nucleic acid according to the present invention, for example, by using computer programs intended for that purpose such as Primer (Version 0.5© 1991, Whitehead Institute for Biomedical Research, Cambridge, Mass.).

Methods for preparing and using probes and primers are described, for example, in Sambrook et al. Molecular Cloning: A Laboratory Manual, 2nd ed, vol. 1-3, ed Sambrook et al. Cold Spring Harbour Laboratory Press, Cold Spring Harbour, N.Y., 1989.

“Probes” are single-stranded nucleic acid molecules with a known nucleotide sequence which is labelled in some way (for example, radioactively, fluorescently or immunologically), which are used to find and mark a target DNA or RNA sequence by hybridising to it.

The term “vector” as used herein encompasses both cloning and expression vectors. Vectors are often recombinant molecules containing nucleic acid molecules from several sources.

A “cloning vector” refers to a nucleic acid molecule originating or derived from a virus, a plasmid or a cell of a higher organism into which another exogenous (foreign) nucleic acid molecule of interest, of appropriate size can be integrated without loss of the vector's capacity for self-replication. Thus vectors can be used to introduce at least one foreign nucleic acid molecule of interest (e.g. gene of interest) into host cells, where the gene can be reproduced in large quantities.

An “expression vector” refers to a cloning vector which also contains the necessary regulatory sequences to allow for transcription and translation of the integrated gene of interest, so that the gene product of the gene can be expressed.

The term “gene” as used herein refers to a nucleic acid molecule comprising an ordered series of nucleotides that encodes a gene product (i.e. specific protein).

The term “protein (or polypeptide or peptide)” refers to a protein encoded by the nucleic acid molecule of the invention, including fragments, mutations and homologs or analogs having the same biological activity i.e. ovulation manipulation activity. The protein or polypeptide or peptide of the invention can be isolated from a natural source, produced by the expression of a recombinant nucleic acid molecule, or can be chemically synthesized.

The term “host cell” refers to a cell which is capable of containing a vector or construct and supports the replication and/or expression of the vector or construct. Suitable hosts cells may be prokaryotic cells such as bacteria, or eukaryotic cells such as yeast, insect, amphibian, or mammalian cells. Preferably, the host cells are bacterial cells.

Understandably, the term “host cell” should also be taken to include a transgenic organism which comprises a host cell.

The term “hybridisation” or grammatical variants thereof means the process of joining two complementary strands of DNA or one each of DNA and RNA to form a double stranded molecule.

The term “construct” as used herein refers to an artificially assembled or isolated nucleic acid molecule which includes the gene of interest. In general a construct may include the gene or genes of interest and appropriate regulatory sequences. It should be appreciated that the inclusion of regulatory sequences in a construct is optional for example, such sequences may not be required in situations where the regulatory sequences of a host cell are to be used. The term construct includes vectors but should not be seen as being limited thereto.

The term “nucleic acid amplification technique” as used herein may generally be considered to refer to polymerase chain reaction or PCR however; it may equally refer to other equivalent techniques for amplifying nucleic acids known to those skilled in the art.

The term ‘variant’ as used herein refers to a nucleic acid molecule or polypeptide wherein the nucleotide or amino acid sequence exhibits substantially 70, 80, 95, or 99% homology with the nucleotide or amino acid sequence as set forth in the sequence listing—as assessed by GAP or BESTFIT (nucleotides and peptides), or BLASTP (peptides), or BLASTX (nucleotides). It should be appreciated that the variant may result from a modification of the native nucleotide or amino acid sequences, or by modifications including insertion, substitution or deletion of one or more nucleotides or amino acids. Where such a variant is desired, the nucleotide sequence of the native DNA may be altered appropriately for example by systhensis of the DNA de novo, or by modification of the native DNA, for example by site-specific or cassette mutagenesis. Preferably, where portions of the cDNA or genomic DNA require sequence modifications, site-specific primer directed mutagenesis is employed using techniques standard in the art. Alternatively, a variant may be naturally occurring. The term variant also encompasses homologus sequences which hybridise under stringent conditions to the sequences of the invention.

The term ‘variant’ also encompasses “conservative substitutions” wherein the alteration of the nucleotide or amino acid sequences, as set out in the sequence listing of this specification, results in the substitution of a functionally similar amino acid residue—see Creighton (1984).

The term ‘fragment nucleic acid molecule’ as used herein refers to a nucleic acid molecule which represents a portion of the nucleic acid molecule of the present invention and is therefore less than full length and comprises at least a minimum sequence capable of hybridising stringently with a nucleic acid molecule of the present invention (or a sequence complementary thereto).

A ‘fragment polypeptide’ as used herein refers to a fragment of a polypeptide which represents a portion of the polypeptide of the present invention and is therefore less than full length and comprises at least a minimum sequence capable of hybridising stringently with a polypeptide of the present invention (or a sequence complementary thereto).

The term “isolated” means substantially separated or purified away from contaminating sequences in the cell or organism in which the nucleic acid naturally occurs and includes nucleic acids purified by standard purification techniques as well as nucleic acids prepared by recombinant technology, including PCR technology, and those chemically synthesised.

The term ‘functional’ refers to either: a nucleic acid molecule which encodes a polypeptide capable of acting as an enzyme in the indole diterpene biosynthetic pathway; or a polypeptide which is capable of acting as an enzyme in the indole diterpene biosynthetic pathway.

Stringent hybridization conditions is a term of art understood by those of ordinary skill in the art. For any given nucleic acid sequence, stringent hybridization conditions are those conditions of temperature, chaotrophic acids, buffer, and ionic strength which will permit hybridization of that nucleic acid sequence to its complementary sequence and not to substantially different sequences. The exact conditions which constitute “stringent conditions” depend upon the nature of the nucleic acid sequence, the length of the sequence, and the frequency of occurrence of subsets of that sequence within other non-identical sequences. By varying hybridization conditions from a level of stringency at which non-specific hybridization occurs to a level at which only specific hybridization is observed, one of ordinary skill in the art can, without undue experimentation, determine conditions which will allow a given sequence to hybridize only with complemtary sequences. Suitable ranges of such stringency conditions are described in Krause and Aaronson (1991). Hybridization conditions, depending upon the length and commonality of a sequence, may include temperatures of 20° C.-65° C. and ionic strengths from 5× to 0.1×SSC. Highly stringent hybridization conditions may include temperatures as low as 40-42° C. (when denaturants such as formamide are included) or up to 60-65° C. in ionic strengths as low as 0.1×SSC. These ranges, however, are only illustrative and, depending upon the nature of the target sequence, and possible future technological developments, may be more stringent than necessary. Less than stringent conditions are employed to isolate nucleic acid sequences which are substantially similar, allelic or homologous to any given sequence.

The preferred nucleic acid molecules and polypeptides of the present invention methods, and uses of same may have a number of utilities which can include:
Manipulating the indole diterpene biosynthetic pathway.
Producing indole diterpene(s), lolitrem(s), enzyme(s), intermediate(s) or other compound(s), associated with the indole diterpene biosynthetic pathway.
Identifying mutations of these genes in endophytes which:
   Do not produce, or produce insufficient levels of lolitrem B, to exhibit toxic effects such as ryegrass staggers; or
   Provide increased insecticidal activity.
Providing nucleic acid molecules which can be used in constructs for expression of lolitrems or other indole diterpenes or intermediate compounds involved in the indole diterpene biosynthetic pathway.

It should also be appreciated from the above description that there is provided nucleic acid molecules for the biosynthesis of indole diterpene compounds. It will be appreciated further that through knowledge of these molecules, further molecules can be determined that relate to various aspects of the biosynthesis process. Further, it will be appreciated that the genes have a variety of resulting applications such as screening to determine biosynthesis products and manipulation of the genes to create desireable intermediate and end product indole diterpene compounds.

Sequence Listings:

| ID No. | Corresponding sequence | Corresponding Figure |
|---|---|---|
| 1. | ItmG nt (N. lolii) | |
| 2. | ItmG pp (N. lolii) | |
| 3. | ItmM nt (N. lolii) | |
| 4. | ItmM pp (N. lolii) | |
| 5. | ItmK nt (N. lolii) | |
| 6. | ItmK pp (N. lolii) | |
| 7. | ItmC nt (N. lolii) | |
| 8. | ItmC pp (N. lolii) | |
| 9. | ItmP nt (N. lolii) | |
| 10. | ItmP pp (N. lolii) | |
| 11. | ItmJ nt (N. lolii) | |
| 12. | ItmJ pp (N. lolii) | |
| 13. | ItmQ nt (N. lolii) | |
| 14. | ItmQ pp (N. lolii) | |
| 15. | ItmD nt (N. lolii) | |
| 16. | ItmD pp (N. lolii) | |
| 17. | ItmG nt (E. festucae) | |
| 18. | ItmG pp (E. festucae) | |
| 19. | ItmM nt (E. festucae) | |
| 20. | ItmM pp (E. festucae) | |
| 21. | ItmK nt (E. festucae) | |
| 22. | ItmK pp (E. festucae) | |
| 23. | Cluster 1 | |
| 24. | Cluster 2 | |
| 25. | Cluster 3 | |
| 26. | Primer ggpps27 | Table 2 |
| 27. | Primer ggpps28 | Table 2 |
| 28. | Primer ggpps29 | Table 2 |
| 29. | Primer CY 4 | Table 2 |
| 30. | Primer CY 5 | Table 2 |
| 31. | Primer lol 1 | Table 2 |
| 32. | Primer lol 2 | Table 2 |
| 33. | Primer lol 3 | Table 2 |
| 34. | Primer lol 7 | Table 2 |
| 35. | Primer lol 14 | Table 2 |
| 36. | Primer lol 15 | Table 2 |
| 37. | Primer lol 17 | Table 2 |
| 38. | Primer lol 18 | Table 2 |
| 39. | Primer lol 28 | Table 2 |
| 40. | Primer lol 29 | Table 2 |
| 41. | Primer lol 32 | Table 2 |

| ID No. | Corresponding sequence | Corresponding Figure |
|---|---|---|
| 42. | Primer lol 34 | Table 2 |
| 43. | Primer lol 35 | Table 2 |
| 44. | Primer lol 43 | Table 2 |
| 45. | Primer lol 48 | Table 2 |
| 46. | Primer lol 49 | Table 2 |
| 47. | Primer lol 63 | Table 2 |
| 48. | Primer lol 79 | Table 2 |
| 49. | Primer lol 135 | Table 2 |
| 50. | Primer lol 147 | Table 2 |
| 51. | Primer lol 148 | Table 2 |

### BRIEF DESCRIPTION OF DRAWINGS

Further aspects of the present invention will become apparent from the following description which is given by way of example only and with reference to the accompanying drawings in which:

FIG. 1. Structure of lolitrem B

FIG. 2. Degenerate PCR and Southern hybridisation of the GGPP synthase gene fragments CY28 and CY29 Degenerate PCR analysis using primers (A) ggpps27 and ggpps28, and (B) ggpps27 and ggpps29. Lane (1) 1 kb+ladder, (2) N. lolii strain Lp19, (3) E. festucae strain Fl1, (4) E. typhina strain E8 (5) wild-type P. paxilli, (6) P. paxilli strain LM662, (7) blank. Southern hybridisation of the ggs fragments. (C) probed with fragment CY29 (ggs1). (D) probed with fragment CY28 (ltmG). Lane (1, 4 and 7) N. lolii strain Lp19, (2, 5 and 8) E. festucae strain Fl1 (3, 6 and 9) E. typhina strain E8. Lanes 1-3 are digested with EcoRI, lanes 4-6 are digested with Hindlll and lanes 7-9 are digested with Sstl. The size standards are in kb.

FIG. 3. N. lolii and E. festucae lolitrem gene cluster. Physical map of the (A) Lp19 and (B) Fl1 lolitrem gene cluster. The CY28 PCR fragment used as a probe to isolate lambda clones, is a green box. Each gene is shown as a black rectangle with intron marked and an arrow above the genes shows the gene direction. The yellow box is a microsatellite with a core sequence of TMTG. The red and blue boxes are the fragments used to make the ltmM knockout construct. The retrotransposons, Tahi and Rua, are shown as red and blue lines with arrow heads as the LTR sequences. Each fragment used as a probe is a green oval placed under the region of the probe. (C) The ltmM knockout construct, pCY39. (D) The PCR screen for a knockout in Fl1. Lanes (1) 1kb+ ladder, (2) CYFl1M-28, (3) CYFl1M-142, (4) CYFl1M-61, (5) CYFl1M-151, (6) Fl1, (7) pCY39, (8) H₂O control. The 7-kb Xhol fragment used for preparing the complementation construct is also shown.

FIG. 4. The nucleotide sequence of N. lolii strain LP19 ltmG.

FIG. 5. The polypeptide sequence of N. lolii strain LP19 LtmG.

FIG. 6. The nucleotide sequence of N. lolii strain LP19 ltmM.

FIG. 7. The polypeptide sequence of N. lolii strain LP19 LtmM.

FIG. 8. The nucleotide sequence of N. lolii strain LP19 ltmK.

FIG. 9. The polypeptide sequence of N. lolii strain LP19 LtmK.

FIG. 10. The nucleotide sequence of N. lolii strain LP19 ltmG, ltmM and ltmK gene cluster.

FIG. 11. The nucleotide sequence of E. festucae strain Fl1 ltmG.

FIG. 12. The nucleotide sequence of E. festucae strain Fl1 ltmM.

FIG. 13. The nucleotide sequence of E. festucae strain Fl1 ltmK.

FIG. 14. The polypeptide sequence of E. festucae strain Fl1 LtmG

FIG. 15. The polypeptide sequence of E. festucae strain Fl1 LtmM

FIG. 16. The polypeptide sequence of E. festucae strain Fl1 LtmK

FIG. 17. HPLC analysis of lolitrem alkaloids in leaf extracts of endophyte infected perennial ryegrass. Pseudostem tissue was harvested two months post-infection and analysed for lolitrems by normal phase HPLC. (A) lolitrem B standard (8.4 μg). (B) wild-type strain Fl1 (plant G1137). (C) ltmM mutant PN2303 (plant G1114). (D) ltmM mutant PN2296 (plant G1126). (E) ltmMG mutant PN2301 (plant G1119). (F) ectopic transformant PN2294 (plant G1130). The y-axis shows fluorescence units in millivolts at A440 nm and the x-axis retention time in min. The peak at retention time of 1.9 min corresponds to the solvent front.

FIG. 18. Structure of paspaline.

FIG. 19. An EST derived nucleic acid fragment from the suppressive subtractive hybridization library with homology to Penicillium paxili paxP

FIG. 20. An EST derived nucleic acid fragment from the suppressive subtractive hybridization library with homology to Penicillium paxili paxP

FIG. 21. An EST derived nucleic acid fragment from the suppressive subtractive hybridization library with homology to Penicillium paxili paxP

FIG. 22. An EST derived nucleic acid fragment from the suppressive subtractive hybridization library with homology to Penicillium paxili paxD

FIG. 23. An EST derived nucleic acid fragment from the suppressive subtractive hybridization library with homology to Penicillium paxili paxD

FIG. 24. An EST derived nucleic acid fragment from the suppressive subtractive hybridization library with homology to Penicillium paxili paxD

FIG. 25. An EST derived nucleic acid fragment from the an vitro culture library with homology to cytochrome P450 monooxygenases

FIG. 26. Schematic diagram of PaxP, showing the placement of the EST sequences. The polypeptide sequence is represented as green blocks with the size indicated in amino acid residues underneath. The intron placements are numbered above the polypeptide. The primers used for PCR amplification are positioned above the region used for primer design. The EST sequences that are part of the ltmP or the ltmJ gene are shown as lines below the EST positions. The EST identification numbers (Table 6) have been reduced to the last three numbers.

FIG. 27. A physical and genetic map of the LP19 ltm cluster 2 locus. The five ltm genes are shown as green arrows, the exons of the ltm genes are blue boxes under the gene. Selected lambda clones isolated with the ltmC and ltmP probes are black lines. The fragments used as probes to isolate the lambda clones are shown as red boxes above the restriction enzyme map. The fragments initially isolated by IPCR are boxed in red.

FIG. 28. The nucleotide sequence of N. lolii strain Lp19, cluster 2, ltmP-rev, ltmQ, ltmD, ltmC-rev, ltm25.

FIG. 29. The nucleotide sequence of N. lolli strain LP19 ltmC.

FIG. 30. The polypeptide sequence of N. lolli strain LP19 ltmC.

FIG. 31. The nucleotide sequence of N. lolii strain LP19 ltmP.

FIG. 32. The polypeptide sequence of N. lolii strain LP19 ltmP.

FIG. 33. The nucleotide sequence of N. lolii strain LP19 ltmQ.

FIG. 34. The polypeptide sequence of N. lolii strain LP19 ltmQ.

FIG. 35. The nucleotide sequence of N. lolii strain Lp19 ltm25.

FIG. 36. The polypeptide sequence of N. lolii strain Lp19 ltm25

FIG. 37. The nucleotide sequence of N. lolii strain LP19 ltmD.

FIG. 38. The polypeptide sequence of N. lolii strain LP19 ltmD.

FIG. 39. A physical and genetic map of the LP19 ltm cluster 3 locus. The two ltm genes, ltmE and ltmJ, are green arrows, the exons of the ltm genes are blue boxes under the gene. The lambda clone, ACY324, is shown as an arrow. The primers, lol205 and lol206, used for amplification of the probe fragment are above the gene. The fragment used as a probe to isolate the lambda clones is shown as a red box above the restriction enzyme map. The hybridisation with fragments ltmE and a fragment spanning ltmE-ltmJ was used to extend the map towards the left by IPCR using the restriction enzymes Clal, Xbal and Hindlll.

FIG. 40. The nucleotide sequence of N. lolii strain Lp19, ltm cluster 3, ltmE and ltmJ.

FIG. 41. The nucleotide sequence of N. lolii strain LP19 ltmJ.

FIG. 42. The polypeptide sequence of N. lolii strain LP19 ltmJ.

FIG. 43. The nucleotide sequence of N. lolii strain LP19 ltmE.

FIG. 44. The polypeptide sequence of N. lolii strain LP19 ltmE.

FIG. 45. Making the constructs for complementation of the paxC deletion mutant. (A) The pPN1851 construct. (B) The pCY34 construct. The ltmC gene from LP19 was amplified with primers lol235 and lol236, digested with Ncd and EcoRI and subsequently cloned into pPN1851. The paxM promoters in a green box. (C) The 3.5 kb Hindlll fragment from LP19 containing ltmC was cloned into pUC118 resulting in pCY66. The pCY66 plasmid was used with p1199 in a co-transformation of ABC83 protoplasts. (D) The 2.5 kb Bcll fragment from P. paxilli contiaing paxC was cloned into p1199 resulting in pJA8.

FIG. 46. TLC analysis of paxc complementation transformants. Indole-diterpenes were extracted from mycelium grown for 7 days in CDYE+TE at 28° C.

All plasmids were used to transform the paxC deletion mutant, ABC83. The plasmids were as follows; pll99; pCY66 contained ltmC under the control of its native promoter; pJA8 contained paxC under the control of its native promoter; pCY34 contained LP19 ltmC gene under the control of the paxM promoter. The + under the TLC plate indicates the presence of a green band identical in Rf to the paxilline standard, while the + indicates possible paxilline production. 13dP=the mobility of paspaline and 13-desoxypaxilline.

FIG. 47. Autoradiographs of Southern analysis of (A) EcoRI digested; (B) SalI digested, N. lolii strains Lp19, LP5, AR1 and LP14, E. festucae strains FL1 and E189, Neotyphodium species LpTG2 strain Lp1 and E. typhina strain E8 hybridised with 32P-labelled ltmP amplified with primers lol196 and lol198; ltmJ amplified with primers lol205 and lol206, and ltmE amplified with primers lol356 and lol341. The sizes of the hybridising bands are shown in kb. (C) A schematic map of the ltm cluster 2 and 3 region showing the approximate deletions in Lp14, Lp1 and AR1 as determined by Southern Analysis.

### BEST MODES FOR CARRYING OUT THE INVENTION

### EXAMPLE 1

### Isolation of Nucleic Acid Fragments Containing Homology to GGPP Synthases from N. lolii and E. festucae

Fungal strains, E. coli strains, plasmids and lambda clones used in this experiment are described in Table 1.

TABLE 1
Strains, plasmids, and lambda clones.

| | PN | | |
|---|---|---|---|
| Strain | number | Relevant characteristics | Reference |
| Lp19 | PN2191 | Neotyphodium lolii | |
| FI1 | | Epichlo\ddot{e} festucae | |
| E8 | | Epichlo\ddot{e} typhina | |
| CYFI1-M28 | PN2303 | E. festucae ΔItmM:: hph | This study |
| CYFI1-M61 | PN2301 | E. festucae ΔItmMG:: hph | This study |
| CYFI1-M142 | PN2296 | E. festucae ΔItmM:: hph | This study |
| CYFI1-M151 | PN2294 | E. festucae ΔItmM:: hph | This study |
| | | ectopic integration | |
| pCB1004 | | Amp^{R}/Hyg^{R} | Carroll et al |
| | | | 1994 |
| pCY28 | | 209 bp ItmG fragment in | This study |
| | | pGEM-T, Amp^{R} | |
| pCY29 | | 272 bp ggsA fragment in | This study |
| | | pGEM-T, Amp^{R} | |
| pCY39 | | Amp^{R}/Hyg^{R}, ItmM Knockout | This study |
| | | construct | |
| pGEM-T | | Amp^{R} | Promega |
| pGEM-T-easy | | Amp^{R} | Promega |
| pPN1688 | PN1688 | Amp^{R}/Hyg^{R} | This study |
| pUC118 | | Amp^{R} | This study |
| λCY218 | | Lp19λGEM12 containing ItmG | This study |
| λCY255 | | Lp19λGEM12 containing ItmK | This study |
| λCY275 | | Lp19λGEM12 overlapping | This study |
| | | λCY255 | |
| λCY100 | | Lp19λGEM12 containing ggsA | This study |
| G1114 | | Nui ryegrass, CYFI1-M28 | This study |
| G1119 | | Nui ryegrass, CYFI1-M61 | This study |
| G1126 | | Nui ryegrass, CYFI1-M142 | This study |
| G1130 | | Nui ryegrass, CYFI1-M151 | This study |
| G1137 | | Nui ryegrass, FI1 | This study |
| G1138 | | Nui ryegrass, endophyte free | This study |

All bacteria were grown in LB medium overnight at 37° C. For maintenance, the fungal cultures were grown on 2.4% potato dextrose (PD; Difco) agar plates at 22° C. until suitable growth was attained. For DNA isolation, the fungal strains were grown in PD broth at 22° C. for 5-12 days. The protein sequences of the available fungal GGPPS genes from:
Neurospora crassa al-3, (accession number MC13867)(Barbato et al. 1996)
S. cerevisiae Bts1 (accession number MA83662)
P. paxilli paxG (accession number AF279808) (Young et al. 2001), and
Gibberella fujikuroi ggs-1 (accession number CM65644) (Mende et al. 1997) and

ggs-2 (accession number CM75568) (Tudzynski and Hölter 1998) were aligned (Higgins et al. 1994) to determine conserved domains that would be suitable for degenerate primer design. Primers, ggpps27, ggpps28 and ggpps29, were designed to three highly conserved regions taking in to consideration the placement of any known introns. The sequences obtained of these and other primers are shown in Table 2.

TABLE 2
Primer list

| | | |
|---|---|---|
| Name | sequence 5′ → 3′ | amplifies |

| | | |
|---|---|---|
| CY 4 | GCT TGG ATC CGA TAT TGA AGG | hph/BamHl |
| | AGC | |
| CY 5 | TTG GAT CCG GTT CCC GGT CGG | hph/BamHl |
| | CAT | |
| ggpps 27 | CAY MGI GGT CAR GGT ATG GA | dPCR |
| ggpps 28 | TTC ATR TAG TCG TCI CKT ATY | dPCR |
| | TG | |
| ggpps 29 | AAC TTT CCY TCI GTS ARG TCY | dPCR |
| | TC | |
| lol 1 | TGG ATC ATT CGC AGA TAC | ItmG |
| lol 2 | GTG TGA GAT TAA GAC GTO | LHS |
| lol 3 | ACC GAC GCC ATT AAT GAG | ItmG |
| lol 7 | ACT GGG CAT CTT CCA TAG | ItmM/mid |
| lol 14 | ATT AGA GGO ACC GAA CGC | RT-PCR ItmM |
| lol 15 | ATC AAG CTG GCT ATC CTC | ItmP |
| lol 17 | AAA TAA TGG GCA AGG AGC | KO Pstl |
| lol 18 | TGG GAAT TTT GGA AAT GGC | KO Pstl |
| lol 28 | GCT CCT TGC CCA TTA TTT | RT-PCR ItmM |
| lol 29 | GTC TTG ATC GTC TGC ATC | RT-PCR ItmP |
| lol 32 | TGT CCG TGC ATC CAT TGT | ItmP |
| lol 34 | CAT AGA GCT AGC TAG AGT | LHS |
| lol 35 | GTT CGG TGC CTC TAA TAC | ItmM/mid |
| lol 43 | GAG GAT AGC CAG CTT GAT | RT-POR ItmP |
| lol 48 | GAT TGG TAC CTT GAA GTC GCT | KO Kpnl |
| | AGT | |
| lol 49 | GTA GGG TAC CTC TAG TAC TGC | KO Kpnl |
| | CTC T | |
| lol 63 | TAG CGA ATC ATT GCG TCG | RT-PCR ItmP |
| lol 79 | ATG GCT GCC AAT GAC TTT CC | RT-PCR ItmG |
| lol 135 | AGG CCA TTT TCG ACA GTT GT | KO integration |
| lol 147 | CCA GCA AGC ATG CAC ATT AC | RHS |
| lol 148 | TGC GTG AGA GAT AAA GCA AG | KO integration |
| pUC | GCC AGG GTT TTC CCA GTC | |
| forward | ACG A | |
| pUChph 3 | CTG CAT CAT CGA AAT TGC | hph |
| pUChph 4 | AAA CCG AAC TGC CCG CTG TTC | hph |
| PUC | GAG CGG ATA ACA ATT TCA | |
| reverse | CAC AGG | |
| T7 | TAA TAC GAC TCA CTA TAG GG | |

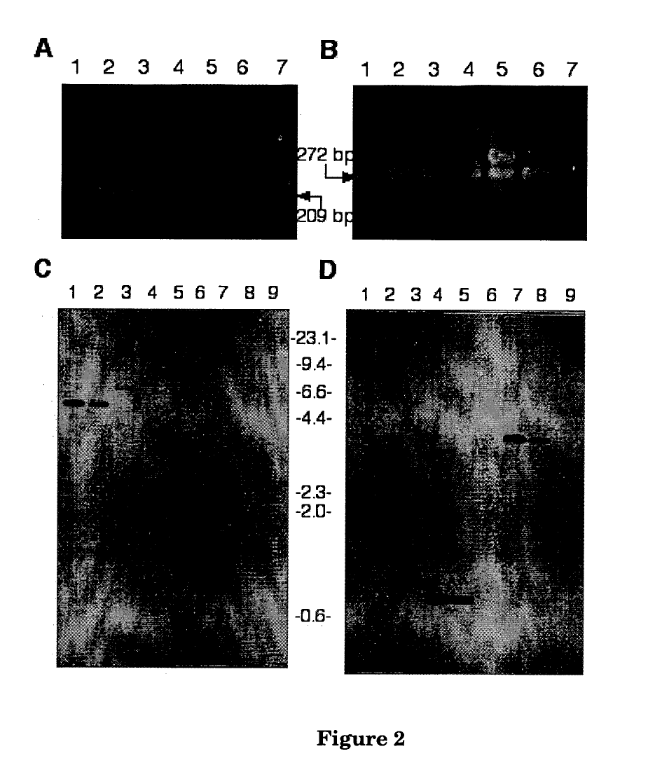

Using degenerate primers designed to fungal GGPP synthase genes, a fragment of the expected size (FIG. 2A) was amplified from lolitrem producing strains, Neotyphodium lolii Lp19, and Epichloë festucae Fl1, and from the lolitrem non-producing strain E. typhina E8. P. paxilli genomic DNA was used as a positive control where two fragments of 330 and 270 bp were amplified, corresponding to paxG, with an intron, and ggs1, without an intron (FIG. 2B). Degenerate PCR amplification was performed using primer pairs ggpps27/ggpps28 and ggpps27/ggpps29 with 5 ng of genomic DNA and 4.8 μM of each primer. The amplification conditions were 95° C. for 2 min followed by 30 cycles of 95° C. for 30 sec, 45° C. for 30 sec and 72° C. for 1 min, then 1 cycle of 72° C. for 5 min. The annealing temperature was also increased to 47° C. with a similar amplification result. The resulting products were cloned into pGEM-T easy (Promega). Plasmid DNA was isolated using a BioRad plasmid mini preparation kit. PCR products were purified using a Qiagen PCR purification kit. Fragments were extracted from agarose using the Qiagen gel extraction kit.

The cloned fragments were distinguished using RFLP analysis by amplifying with primers ggpps27 and ggpps28 using standard PCR conditions. The resulting fragments were digested with an appropriate enzyme (NotI and Sau3AI) and resolved on a 2% agarose gel.

The LP19 PCR product amplified with primer set ggpps27 and ggpps29 was cloned into pGEM-T easy and sequenced. DNA fragments were sequenced by the dideoxynucleotide chain termination method (Sanger et al. 1977) using Big-Dye (Version 3) chemistry with oligonucleotide primers (Sigma Genosys) to pGEM-T easy, N. lolii and E. festucae sequences. Products were separated on an ABI Prism 377 sequencer (Perkin-Elmer).

Sequence data was assembled into contigs using SEQUENCHER version 4.1 (Gene Codes) and analyzed using the Wisconsin Package version 9.1 (Genetics Computer Group, Madison, Wis.). Sequence comparisons were performed through Internet Explorer version 6.0 at the National Center for Biotechnology Information (NCBI) site (http://www.ncbi.nlm.nih.gov/) using the Brookhaven (PDB), SWISSPROT and GenBank (CDS translation), PIR and PRF databases employing algorithms for both local (BLASTX and BLASTP) and global (FASTA) alignments (Pearson and Lipman 1988; Altschul et al. 1990; Altschul et al. 1997).

A BLASTX of the CY29 sequence, showed high sequence similarity (E value of 7e-41) to the N. crassa GGPPS (accession number MC13867) and other GGPPS sequences.

An RFLP screen of the remaining clones revealed a second unique fragment, CY28, that also shows strong similarity to GGPPS genes (the top score was to P. paxilli Ppggs1 accession number AF279807, Young et al 2001).

CY28 was amplified with ggpps27 and ggpps28 and is therefore a shorter product than the CY29 fragment. The two sequences, CY28 and CY29, share 61.7% identity to each other at the DNA level.

To determine which clone is the GGPP synthase involved in lolitrem biosynthesis, each fragment was hybridized to genomic DNA from the two lolitrem producing strains, LP19 and Fl1, and the non-producing E8 strain. DNA was transferred to positively charged nylon membrane (Roche) using standard techniques (Sambrook et al. 1989). Fragments required for radioactive probes were amplified using primer pairs stated in Table 3 below.

Each probe fragment was purified using a Qiagen PCR purification kit and 30 ng of DNA was [α-³²P]-dCTP radiolabelled using HighPrime (Roche). The labeled probes were spun through a Pharmacia ProbeQuant column before hybridisation. Hybridisations were performed overnight at 65° C. and the filters were washed in 2×SSC, 0.1% SDS at 50° C.

TABLE 3
Primer combinations for hybridisation probes and RT-PCR analysis

| | | | Size bp | |
|---|---|---|---|---|
| Gene | primer 1 (5′) | primer 2 (3′) | genomic (cDNA) | introns amplified |
| CY28 | g27 | g28 | 209 | |
| CY29 | g27 | g29 | 272 | |
| ItmG | lol3 | lol1 | 407 (353) | 2 |
| ItmM | lol7 | lol35 | 448 (382) | 1 |
| ItmK | lol33 | lol37 | 3277 | |
| ItmK | lol15 | lol32 | 416 (365) | 5 |
| ItmG | lol79 | lol1 | 630 (525) | 1, 2 |
| ItmM | lol7 | lol35 | 448 (382) | 1 |
| ItmM | lol14 | lol28 | 576 (414) | 2, 3 |
| ItmK | lol29 | lol15 | 1122 (816) | 1, 2, 3, 4, 5 |
| ItmK | lol43 | lol63 | 839 (684) | 6, 7 |

The hybridising patterns (FIGS. 2C & D) showed that CY29 hybridized to all three strains while CY28 hybridised just to the two lolitrem producers, LP19 and Fl1. This data indicates that CY29 is the orthologue of P. paxilli paxG and CY28 the orthologue of P. paxilli ggs1.

For reference these genes are named NlggsA and NlltmG respectively (ltm=lolitrem biosynthesis).

### EXAMPLE 2

### Isolation of Genomic Fragments Corresponding to ltm Genes

Using degenerate primers designed to fungal GGPP synthase genes, a fragment of the expected size (Figure2A) was amplified from lolitrem producing strains, Neotyphodium lolii Lp19, and Epichloe festucae Fl1, and from the lolitrem non-producing strain E. typhina E8 lolitrem non-producing strain. P. paxilli genomic DNA was used as a positive control where two fragments of 330 and 270 bp were amplified, corresponding to paxG, with an intron, and ggs1, without an intron (FIG. 2B).

The LP19 PCR product amplified with primer set ggpps27 and ggpps29 was cloned into pGEM-T easy and sequenced. A BlastX analysis of the CY29 sequence, showed high sequence similarity (E value of 7e-41) to the N. crassa GGPPS (accession number ACC13867) and other GGPPS sequences (Table 4). An RFLP screen of the remaining clones revealed a second unique fragment, CY28, that also showed strong similarity to GGPPS genes (the top score was to P. paxilli Ppggs1 accession number AF279807, Young et al. 2001). CY28 was amplified with ggpps27 and ggpps28 and is therefore a shorter product than the CY29 fragment. The two sequences, CY28 and CY29, share 61.7% identity to each other at the DNA level.

To determine which clone is the GGPP synthase involved in lolitrem biosynthesis, each fragment was hybridized to genomic DNA from the two lolitrem producing strains, LP19 and Fl1, and the non-producing E8 strain. The hybridising patterns (FIGS. 2 C & D) showed that CY29 hybridized to all three strains while CY28 hybridised just to the two lolitrem producers, LP19 and Fl1. This data indicates that CY29 is the orthologue of P. paxilli ggs1 and CY28 the orthologue of P. paxilli paxG.

For reference these genes are named ggsA and ltmG respectively (ltm=lolitrem biosynthesis).

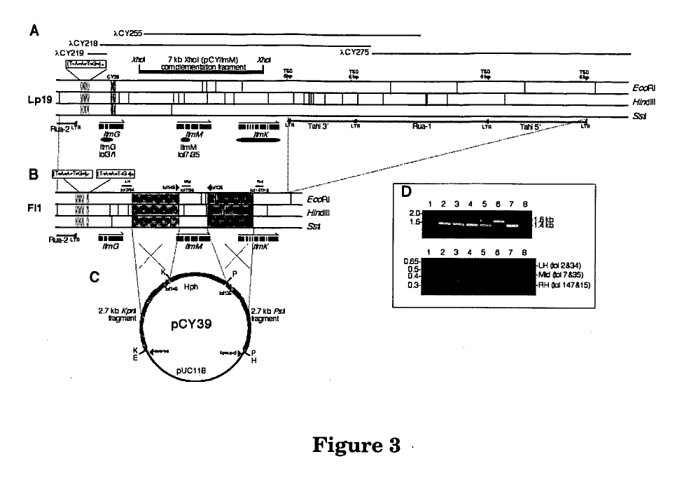

The ltmG fragment, CY28, was used as a probe to isolate sequences from a LP19 λGEM12 genomic library. This region of the genome is under represented in the library with only five clones isolated from ˜80,000 plated. A 15.6-kb lambda clone, λCY218 (FIG. 3), was completely sequenced and shown to contain a complete copy of the ltmG gene.

To obtain further sequence to the left of ltmG, the LP19 λGEM12 library was screened with a probe amplified with primers lol3 and lol1. Hybridization of the library identified one clone λCY219 that contains extra flanking sequence (FIG. 3), however, this clone was severely rearranged and only 1051 bp reflects the correct genomic arrangement. Sequence analysis of ltmG predicts the presence of two introns (FIG. 3). These two introns were confirmed by cDNA analysis with RNA isolated from endophyte infected ryegrass. These introns are conserved in position with two of the four introns found in the ggs-2 gene from G. fujikouri (Tudzynski and Hölter 1998) and two of the three introns found in P. paxilli paxG (Young et al. 2001).

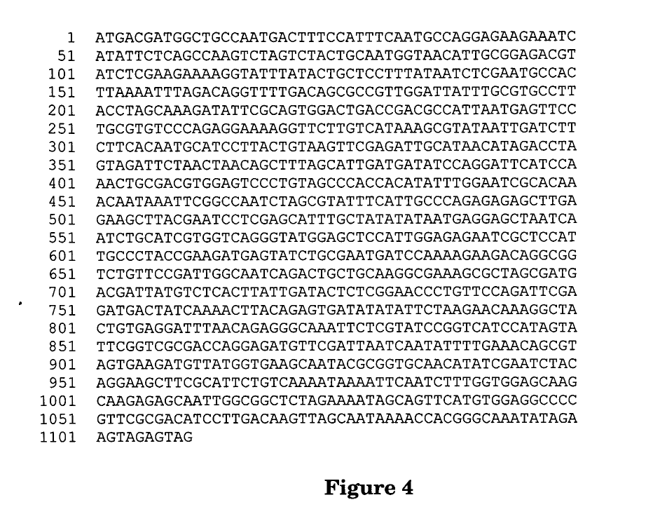

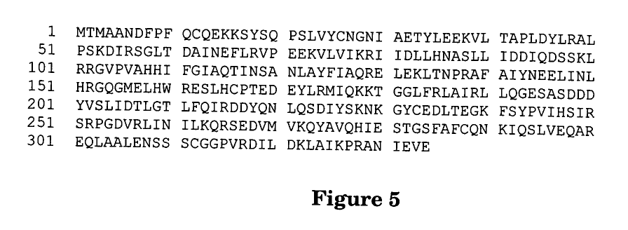

The nucleotide sequence of ltmG from N. lolii strain LP19 is shown in FIG. 4. LtmG is predicted to encode a polypeptide of 334 amino acids with an unmodified molecular weight of 37.9 kDa (Table 4). The amino acid sequence of the deduced N. lolii LtmG polypeptide is shown in FIG. 5. FastA analysis shows that LtmG shares 54.1% and 52.6% identity to N. lolii GgsA and P. paxilli PaxG polypeptide sequences, respectively. LtmG contains the five conserved domains found in all prenyl diphosphate synthases (Chen et al. 1994), including the highly conserved aspartate-rich motifs, DDXXD and DDXXN/D, of domains II and V that are proposed binding sites for the isopentenyl diphosphate (IPP) and the allyl isoprenoid substrates. This analysis suggests that LtmG is a GGPP synthase required for the first commited step in lolitrem biosynthesis.

TABLE 4
Analysis of genes in the lolitrem B biosynthesis cluster

| | | | Tran- | | Ho- | |
|---|---|---|---|---|---|---|
| | | Size | script | Intron | mologous | Protein |
| Gene | Putative activity | (aa) | size | number | pax gene | identity |
| ggsA | Geranylgeranyl | | | 0 | ggs1 | |
| | diphosphate | | | | | |
| | synthase | | | | | |
| ItmG | Geranylgeranyl | 334 | 1002+ | 2 | paxG | 52.6% |
| | diphosphate | | | | | |
| | synthase | | | | | |
| ItmM | FAD dependent | 472 | 1416+ | 3 | paxM | 41.0% |
| | monooxygenase | | | | | |
| ItmK | cytochrome P450 | 533 | 1599+ | 7 | paxP | 31.3% |
| | monooxygenase | | | | | |

### EXAMPLE 3

### Identification of a Gene Cluster for Lolitrem Biosynthesis

Adjacent to ltmG are two genes, ltmM and ltmK, (FIG. 3) proposed to be a FAD-dependent monooxygenase and cytochrome P450 monooxygenase, respectively. Sequence analysis and characterisation by cDNA analysis of the ltmM gene confirms the presence of three introns (FIG. 3).

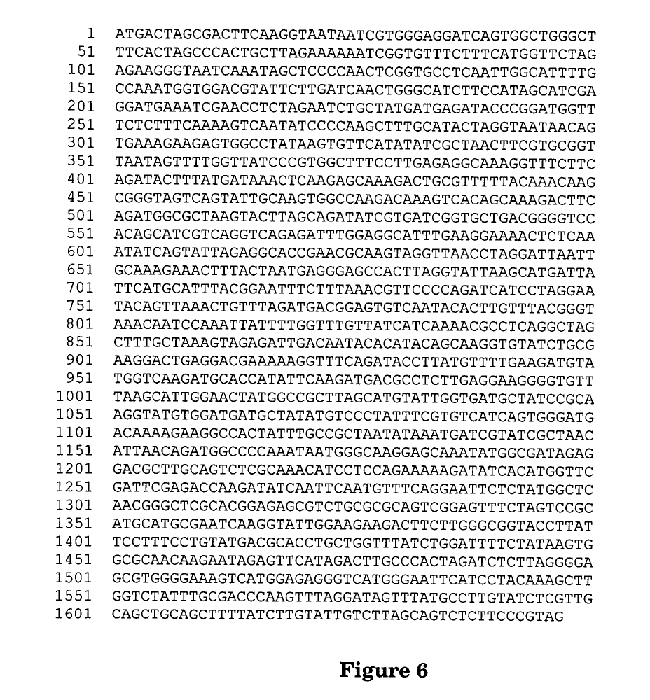

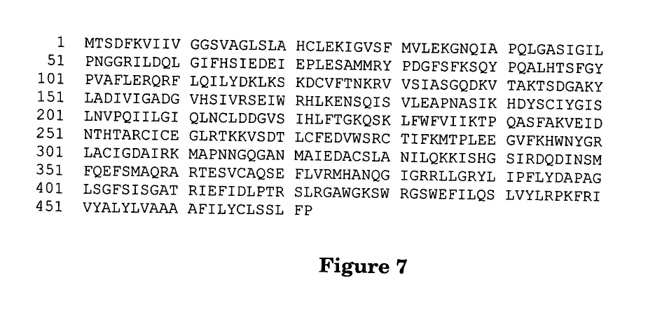

The first two of these introns are conserved with those found in the P. paxilli paxM gene. The third intron is 106 bases, being the largest of the ltm introns confirmed. LtmM is predicted to encode a polypeptide of 472 amino acids with an unmodified molecular weight of 52.5 kDa (Table 4). The nucleotide sequence of N. lolii ltmM and the deduced amino acid sequence of the LtmM polypeptide are shown in FIGS. 6 and 7, respectively. BLASTP analysis showed that LtmM shares 41.0% identity to PaxM from P. paxilli (E value 5e-94). Clustal W alignment (Higgins et al. 1994) of LtmM with PaxM and other closely related polypeptide sequences, identifies the presence of four highly conserved motifs, the dinucleotide binding domain (Wierenga et al. 1986) the ATG motif (Vallon 2000), a GD motif (Eggink et al. 1990) and a G-helix. These motifs are good indicators of a modified Rossman fold, used by many flavoproteins to bind FAD. This analysis suggests that LtmM, like PaxM, is a FAD-dependent monooxygenase, possibly an epoxidase, required for epoxidation of GGPP before cyclisation.

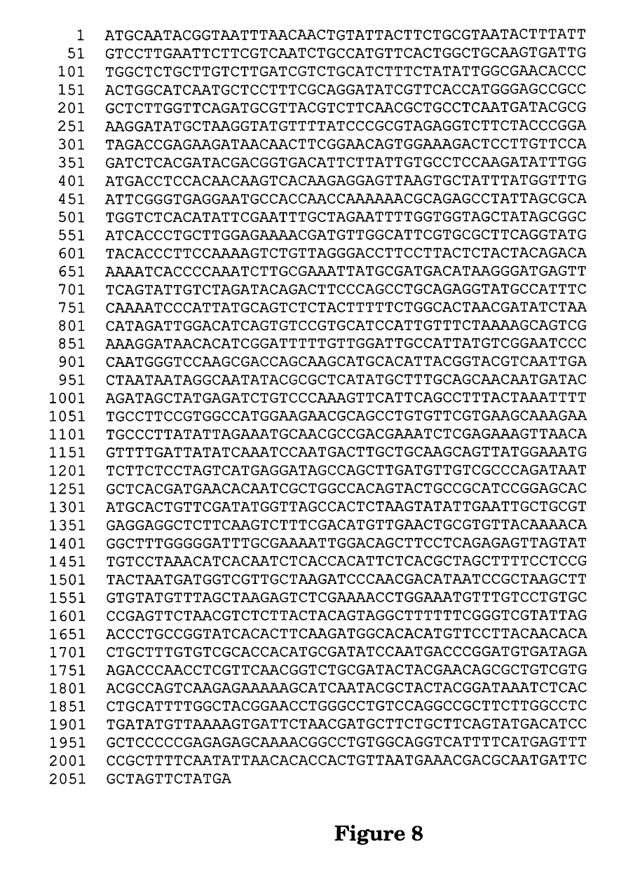

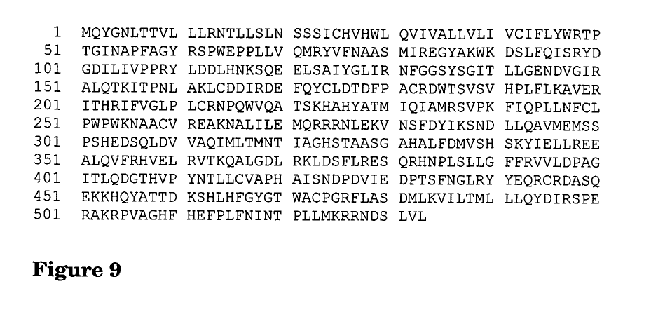

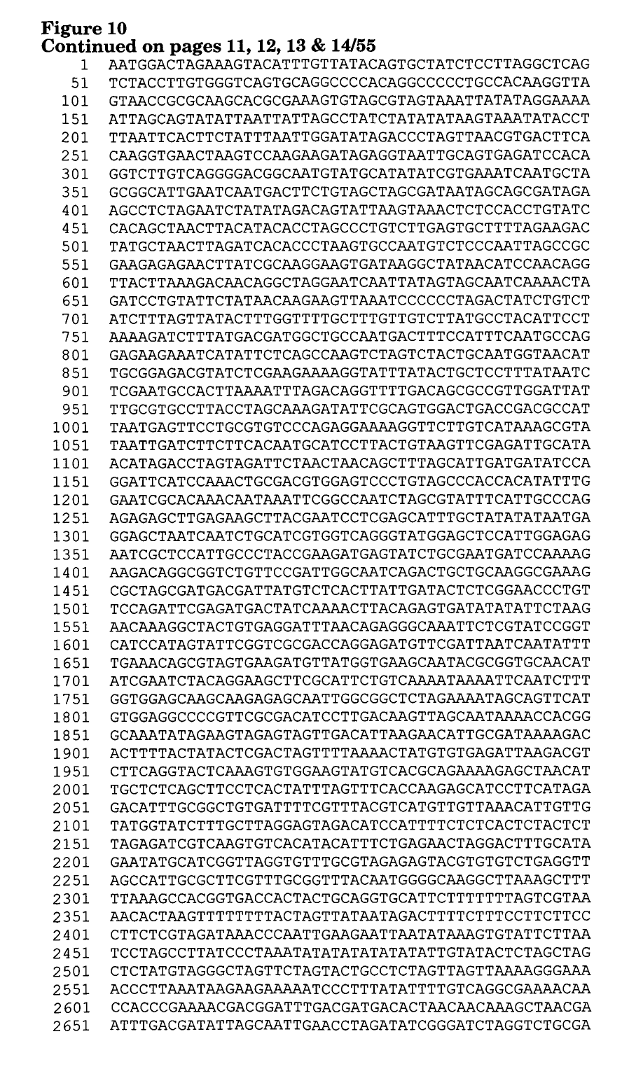

Sequence analysis and characterisation by cDNA analysis of ltmK identified seven introns, four of which are conserved with P. paxilli paxP and three are conserved with P. paxilli paxQ. The nucleotide sequence of N. lolii ltmK and the deduced amino acid sequence of the LtmK polypeptide are shown in FIGS. 8 and 9, respectively.

LtmK is predicted to encode a polypeptide of 533 amino acids with an unmodified molecular weight of 60.9 kDa (Table 4). LtmK contains the classical signature motifs of cytochrome P450 enzymes, including a haem-binding domain (Graham-Lorence and Peterson 1996). However, it does not appear to be an orthologue of either PaxP (E value of 9e-62) or PaxQ (E value of 2e-22), two cytochrome P450 enzymes required for paxilline biosynthesis in P. paxilli (McMillan et al. 2003), as two other cytochrome P450 genes identified from EST sequences have greater similarity to these genes (see below).

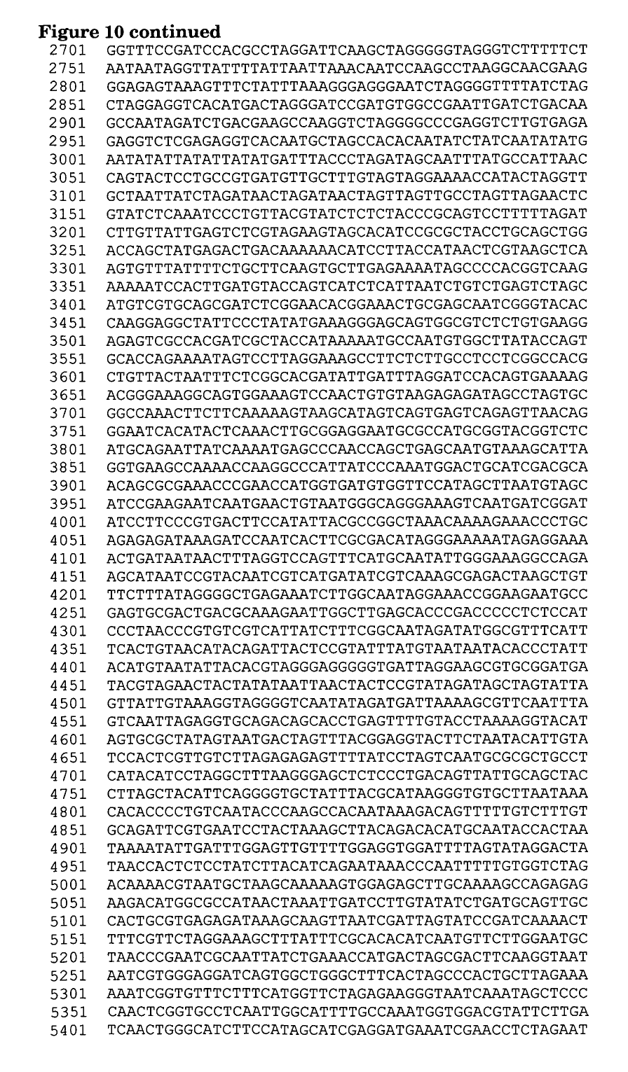

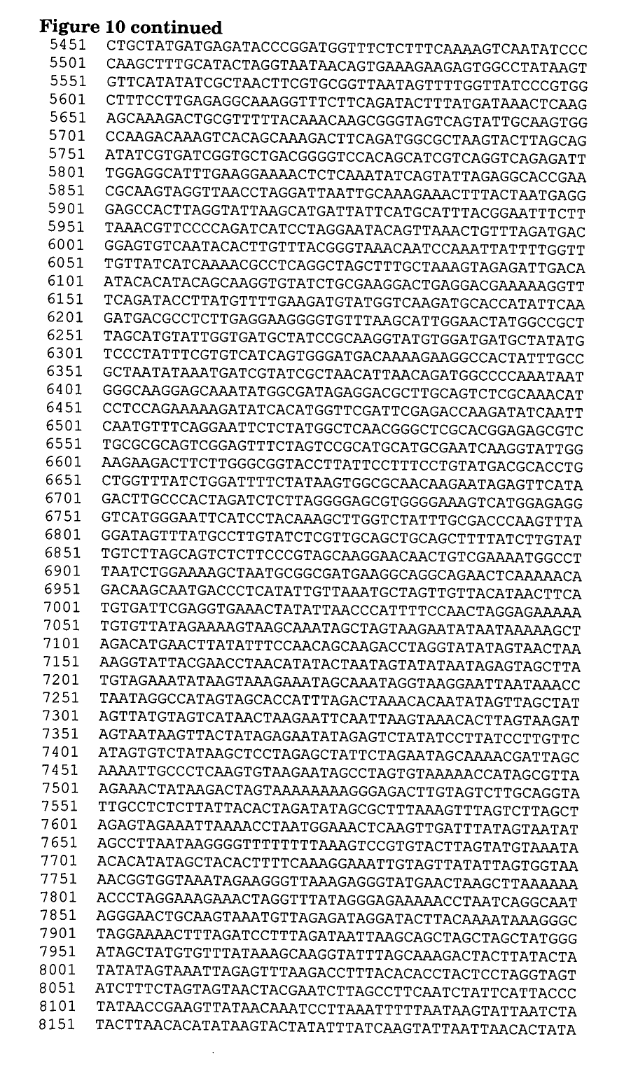

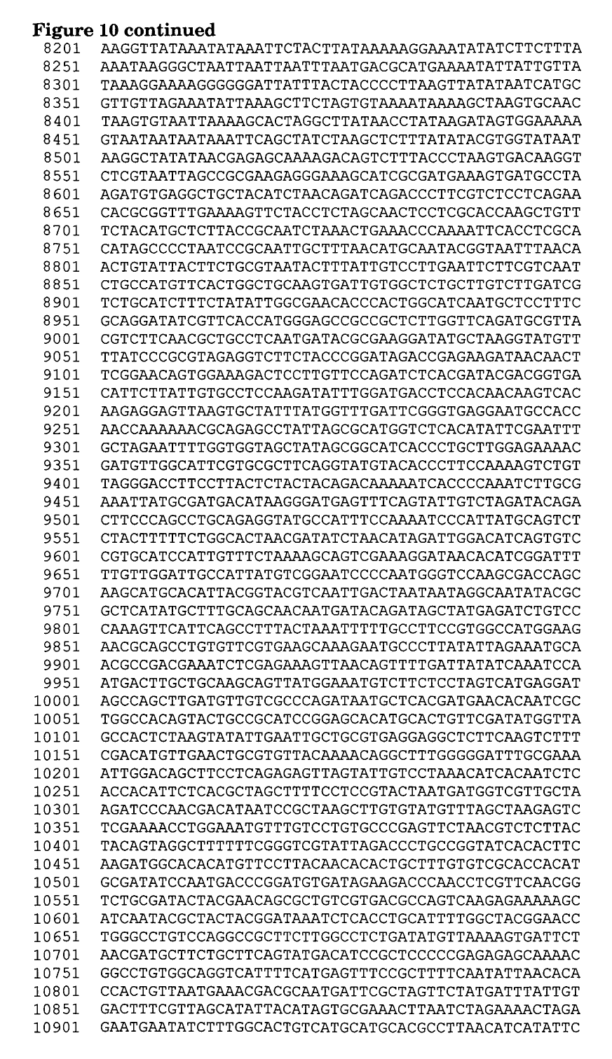

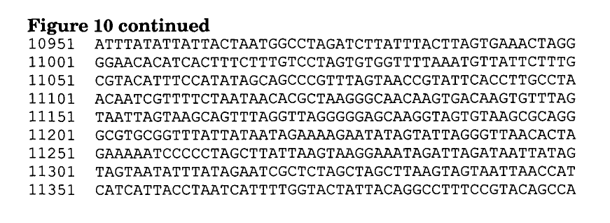

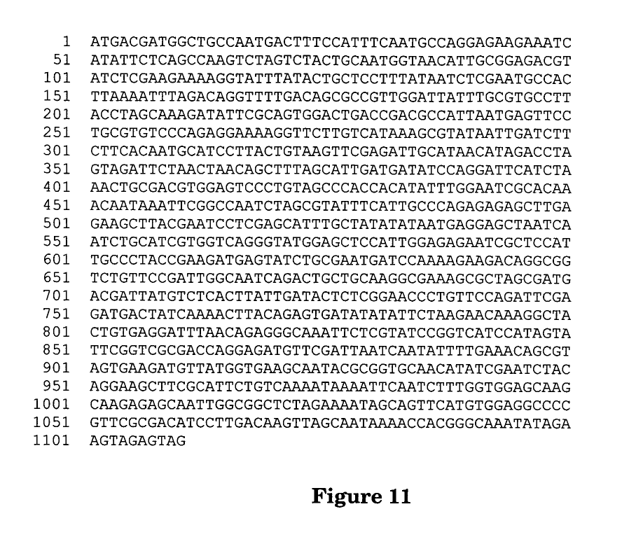

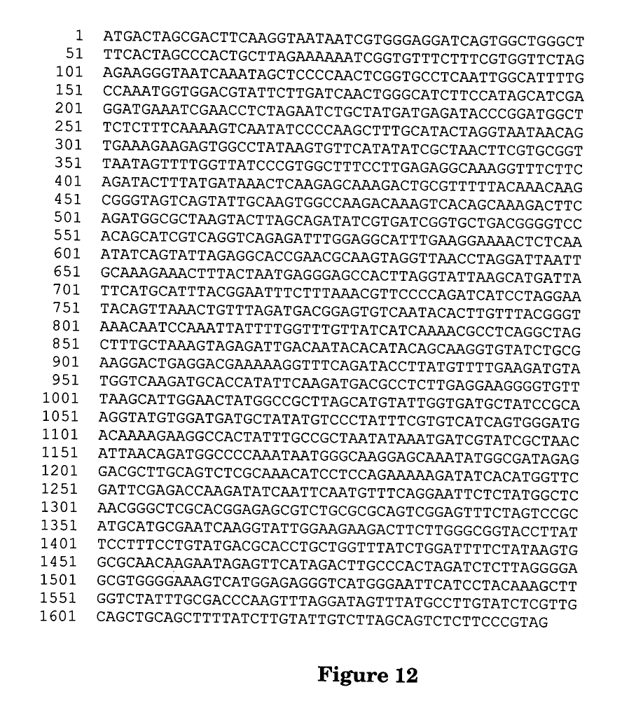

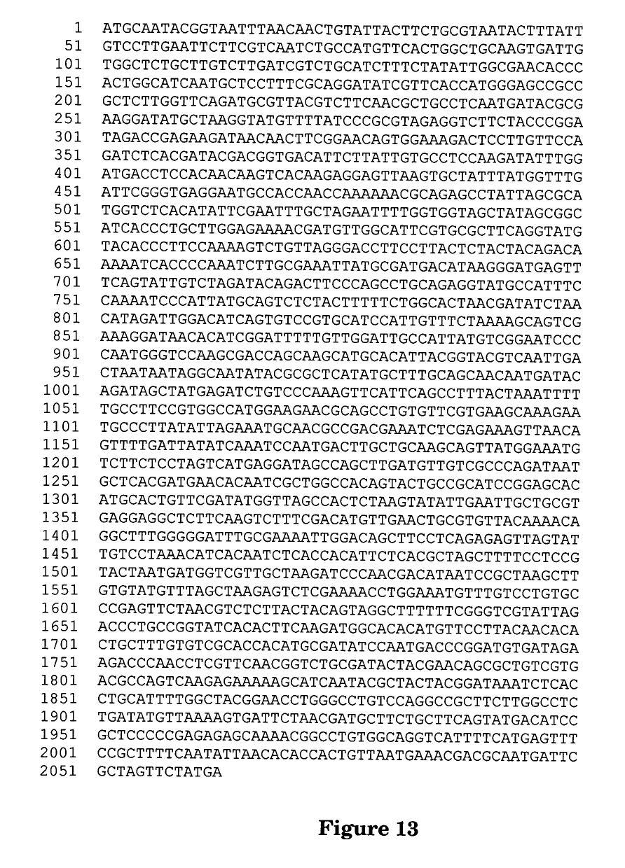

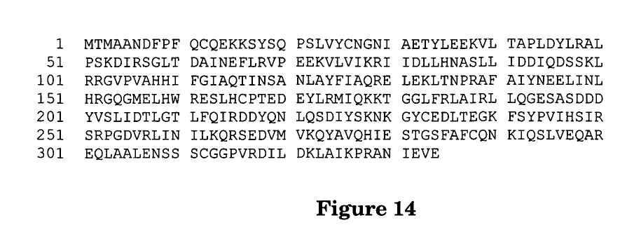

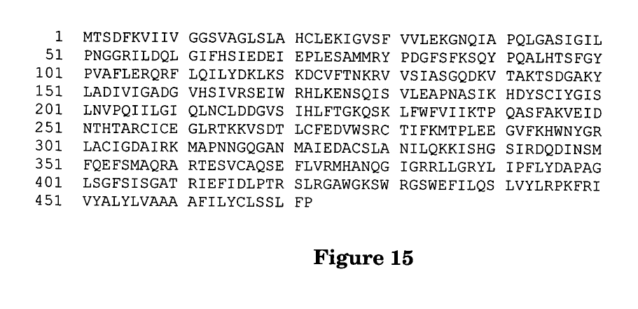

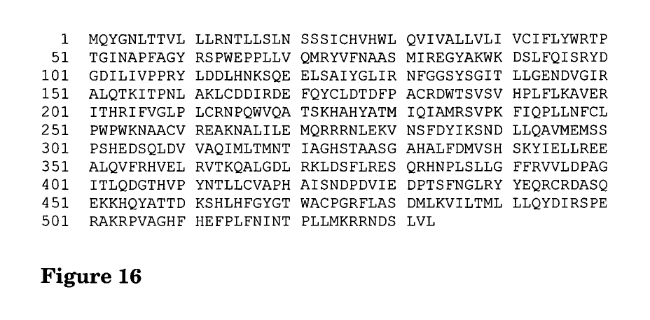

Therefore, ltmG forms a gene cluster with an orthologue of paxM (ltmM) and a cytochrome P450, ltmK, of as yet unknown function in lolitrem biosynthesis. The complete nucleotide sequence of this region is shown in FIG. 10. The corresponding region was sequenced from the E. festucae strain Fl1 and shown to be 99.9% identical to Lp19, at the DNA level, from the start of ltmG to the stop codon of ltmK. The nucleotide sequence of E. festucae ltmG, ltmM and ltmK and the deduced amino acid sequence of the corresponding polypeptides LtmG, LtmM and LtmK are shown in FIGS. 11 to 16, respectively. Comparison of the E. festucae ltmM sequence to N. lolii ltmM shows two base transitions of A→G at base . . . and T→C at base . . . . Only the first transition results in a residue change with a conservative replacement of methionine (in N. lolii ltmM) to valine (in E. festucae ltmM). The promoter region of N. lilii ltmM and E. festucae ltmM have two differences, the first, T→C at base −356 is at a Hindlll site that is absent from E. festucae ltmM and the second is at base −1038 where a GAGA in LP19 has expanded to GAGAGA in Fl1. N. lolii ltmK and E. festucae ltmK are identical in sequence.

The DNA sequence flanking the right-hand end of the ltm gene cluster contains a high AT content (71.2 %) compared to that of the ltm genes at 59.3% AT and, ggsA at 40.9% AT. Blast searches of this flanking region reveal sequence similarity to retrotransposons, however, these sequences are very degenerate and no open reading frames are visible.

### EXAMPLE 4

### Confirmation that ltmM is Essential for Lolitrem B Function by—Deletion of ltmM and Complementation of ltmM Mutant

A gene knockout of ltmM in the E. festucae strain Fl1 was used to confirm that ltmM is essential for lolitrem production. A replacement construct, pCY39, was used in a gene disruption to recombine into the wild-type genome (FIG. 3). An initial PCR screen of 159 hygromycin resistant transformants with primers lol148 and lol135, that amplify both the wild-type ltmM gene (1.6 kb) and the integrating plasmid (1.4 kb) identified replacements of ltmM. Transformants that contain only the integrating plasmid were ‘knockout’ candidates and were screened further. The second PCR screen was with primer sets to the upstream (1012 and 10134: 574 bp), ltmM gene (1017 and lol135: 448 bp), or downstream (lol147 and lol15: 317 bp) regions, where absence of the ltmM gene confirmed the deletion event. Southern analysis was used to distinguish the true knockouts, of which 3.9% (5/159) contained a single integration of the plasmid. During the screen for a homologous recombination event, a transformant, CYFl1-M61, was identified that has a deletion of ltmM and is also deleted beyond ltmG, but the extent of the deletion remains uncharacterised.

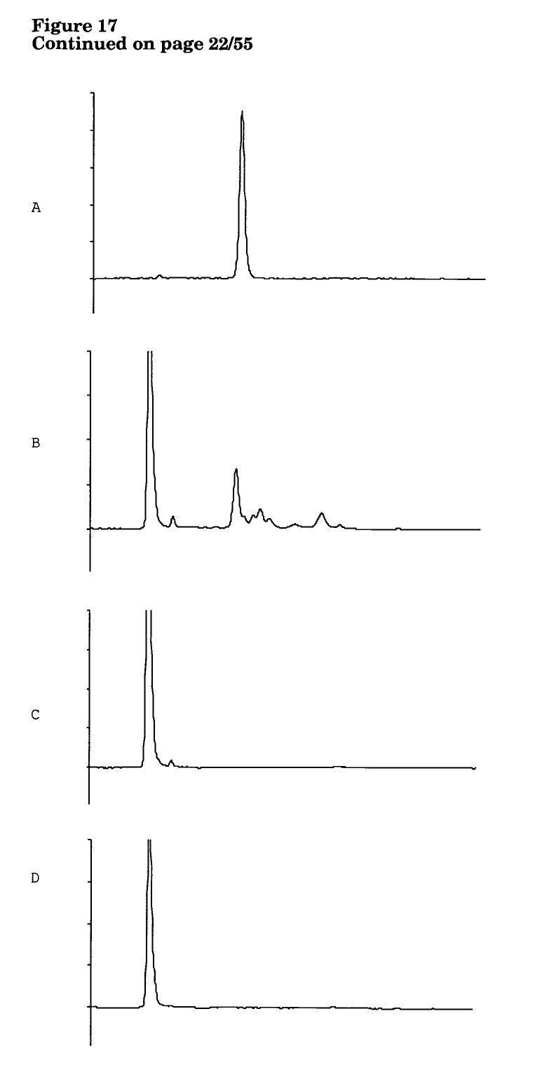

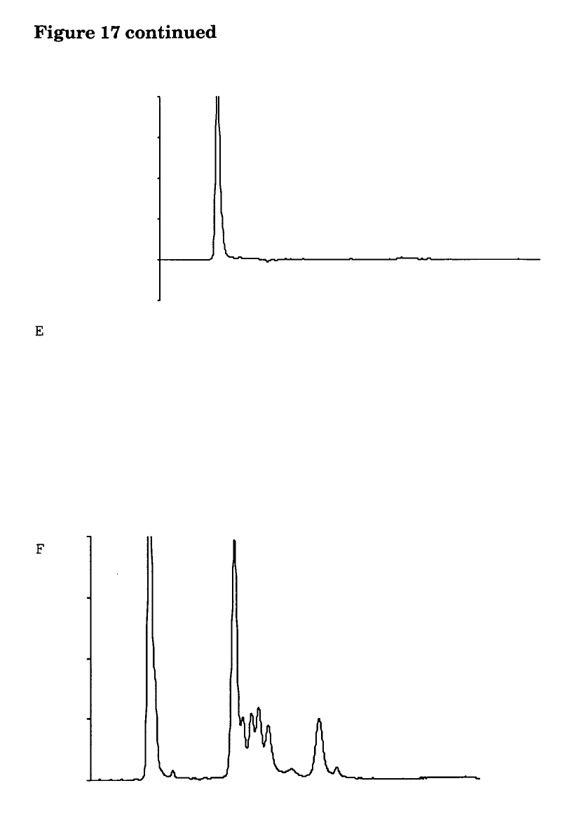

Two independent knockout strains, CYFl1-M28 (PN2303) and CYFl1-M142 (PN2296), the deletion mutant CYFl1-M61 (PN2301), an ectopic mutant CYFl1-M151 (PN2294), and wild-type Fl1 were used to infect endophyte-free perennial ryegrass plants. Each plant was screened for systemic endophyte infection by aniline blue staining confirming normal endophyte associations with the grass. The rate of infection (Table 5) was determined once the plants had established reasonable growth and shows that each strain has a similar infection rate. The endophyte infected plants were grown in a containment green house and were screened for alkaloid production in mid-summer. The alkaloid levels (summarised in Table 5) show that the endophyte strains with a deleted ltmM gene (CYFl1-M28, CYFl1-M61 and CYFl1-M142) are unable to produce lolitrem B (FIG. 17) but the level of ergovaline and peramine production is consistent with wild-type and the ectopic integrant CYFl1-M151. The knockouts (CYFl1-M28 and CYFl1-M142) and deletion mutant (CYFl1-M61) are also devoid of two smaller peaks, that are assumed to be lolitrem A and E, respectively (FIG. 17).

A complementation construct for ltmM, pCYltmM, was made by cloning a 7-kb Xhol fragment containing 2.2 kb of 5′ and 3kb of 3′ ltmM sequences into pll99. Four random integrants of PN2303 containing this construct were infected into plants and shown to synthesize lolitrems.

Plant Inoculation

Two independent knockout strains, CYFl1-M28 and CYFl1-M142, the deletion mutant CYFl1-M61, an ectopic mutant CYFl1-M151, and wild-type Fl1 were used to artificially infect endophyte-free perennial ryegrass plants. Ryegrass cultivar Nui was infected with fungal endophyte according to the procedure of (Latch and Christensen 1985). Four-five weeks after inoculation the plants were checked for systemic endophyte infection by immunoblotting with endophyte antisera and staining pseudostem leaf peels with aniline blue to detect the presence of the endophyte. Plants that were endophyte positive were repotted and allowed to grow under greenhouse conditions. The rate of infection (Table 5) was determined once the plants had established reasonable growth and shows that each strain has a similar infection rate.

Alkaloid Analysis

The endophyte infected plants were grown in a containment green house and were screened for alkaloid production mid-summer. Endophyte infected plant pseudostem material was freeze dried and milled. For lolitrem analysis weighed portions (c. 50 mg) were extracted for 1 hour at ambient temperature with 1 ml of dichloroethane-methanol, 9:1 by volume, in 2 ml polypropylene screw cap vials turning end for end for agitation. The extract was separated by centrifugation and 8 μl portions were examined for lolitrems by normal phase high performance liquid chromatography (Shimadzu LC-10A system) on Alltima silica 5μ 150×4.6 mm columns (Alltech Associates, Deerfield, Ill.). The mobile solvent was dichloromethane-acetonitrile-water, 860:140:1 by volume, with a flow rate of 1 ml/min. Lolitrems were detected by fluorescence (Shimadzu RF-10A, excitation 265 nm, emission 440 nm). Lolitrem B eluted at approximately 4.5 minutes followed by smaller amounts of other lolitrems. The amount of lolitrem B was estimated by comparison of integrated peak areas with external standards of authentic lolitrem B. The detection limit was estimated as <0.1 ppm of lolitrem B.

Ergovaline and peramine were analysed by the method of Spiering et al.(2002). Simplified extraction of ergovaline and peramine for analysis of tissue distribution in endophyte-infected grass tillers. J Agricultural and Food Chem 50:5856-5862.

The alkaloid levels (summarised in Table 5) show that the endophyte strains with a deleted ltmM gene (CYFl1-M28, CYFl1-M61 and CYFl1-M142) are unable to produce lolitrem B (Table 5) but the level of ergovaline and peramine production is consistent with wild-type and the ectopic integrant CYFl1-M151. The knockouts (CYFl1-M28 and CYFl1-M142) and deletion mutant (CYFl1-M61) are also devoid of two smaller peaks, that are assumed to be lolitrem A and E, respectively (Table 5).

TABLE 5
Rates of infection, fungal biomass and alkaloid production

| | | Number of | | | | |
|---|---|---|---|---|---|---|
| | Fungal | plants/ | Infection | Lolitrem | Ergovaline | Peramine |
| Strain | Type¹ | association | Rate² (%) | (ppm) | (ppm) | (ppm) |
| CYFI1M-28 | KO | 5 | 20 | 0 | 0.4-1.3 | 30-40 |
| CYFI1M-61 | Del | 4 | 17 | 0 | 0.7-3.3 | 24-41 |
| CYFI1M-142 | KO | 5 | 17 | 0 | 0.1-2.0 | 14-47 |
| CYFI1M-151 | Ectopic | 5 | 17 | 4.4-16.7 | 0.5-1.2 | 21-55 |
| FI1 | Wt | 4 | 22 | 6.2-12.8 | 0.8-1.5 | 31-66 |
| Endophyte | NA | 3 | NA | 0 | 0 | 0 |
| Free | | | | | | |

¹KO = ItmM knockout, Del = deletion mutant, Wt = Wildtype, NA = Not applicable.
²Infection rates were determined as a percentage of endophyte infected from the surviving plants. The infection rates are low as the endophyte is inserted into young plants at a wound site.

### EXAMPLE 5

### Construction and Sequencing of Suppressive Subtractive Hybridisation Libraries

To identify additional genes involved in the lolitrem biosynthetic pathway an approach of EST sequencing from both N. lolii in vitro culture derived cDNA libraries and from subtracted plant derived cDNA libraries was adopted. ESTs within the libraries derived from N. lolii and with homology to genes from the paxilline biosynthetic pathway are good candidates for orthologous lolitrem biosynthetic genes. It was expected that some genes may be expressed in in vitro cultures but many may only be expressed in planta so the dual approach was taken. The in vitro culture libraries were derived from liquid cultures in both rich and minimal media to increase the chance of identifying ESTs that may only be expressed under starvation conditions and are described in example 6. The subtracted libraries were derived by constructing cDNA from both infected and uninfected perennial ryegrass plants and performing suppressive subtractive hybridization to enrich for fungal cDNAs.

Infected Plant Material

Perennial ryegrass genotypes are genetically complex due to the outbreeding nature of this species. To eliminate plant genotype effects and enable the comparison of infected and uninfected perennial ryegrass plants with identical genetic backgrounds cloned lines of infected Nui were cured of the fungus. The isogenic ryegrass lines infected or uninfected with N. lolii strain LP19 (AR42) were produced as described below. LP19 (AR42) is a novel endophyte from the AgResearch collection and it is known to produce Lolitrem B, Ergovaline and Peramine. LP19 is an endophyte that has been isolated from its parent plant and inoculated into the ryegrass cultivar Nui i.e. it is a novel endophyte.

Positive and negative clones of the above material were produced by taking a positive plant and dividing the tillers up to produce a number of cloned plants. Some of the clones were then treated with a systemic fungicide to eliminate the endophyte.

This was done by striping tillers down and soaking in a 2 g/L solution of Benlate (50% Benomyl w/w) for several hours then planting them in clean river sand saturated with the solution. Pots were watered to weight for several weeks such that the tillers were essentially immersed in fungicide for this period. Plants are potted into commercial potting mix and tillers assayed for endophyte presence. Endophyte free tillers were removed to new pots and tested periodically for endophyte presence to ensure that the fungus has been successfully eliminated. In this way we obtain E+ and E− cloned copies of an individual ryegrass genotype.

Plants were grown in the glasshouse in pots containing commercial potting mix. Plants were dissected in order to provide emerging immature leaf tissue and mature sheath tissue. Material was harvested and frozen immediately at −80C until needed.

Development of Suppressive Subtractive Hybridisation Libraries

RNA was extracted from the harvested plant tissues using the Triazol method (Invitrogen) following the manufacturers recommendations. Messenger RNA was purified from this using mRNA purification kits (Amersham) following the manufacturers recommendations. Messenger RNA (mRNA) was used in subsequent subtractive hybridisations using the Suppressive Subtractive Hybridisation (SSH) kit (Clontech) as per the manufacturer's instructions.

Subtractions were carried out in both a ‘forward’ and ‘reverse’ direction using ‘tester’ and ‘driver’ cDNAs as follows:
Tester equals cDNA from infected plants (Nle+).

Driver equals cDNA from uninfected plants (Nle−).

| Plant line | Leaf tissue | Library |
|---|---|---|
| Nle + M | Mature | Up-regulated |
| Nle − M | Mature | Down-regulated |
| Nle + I | Immature | Up-regulated |
| Nle − I | Immature | Down-regulated |

Subtractions were carried out using tester and driver from both immature and mature tissue and in both directions. Forward subtractions enrich for up-regulated genes and reverse subtractions enrich for down-regulated genes. After the subtraction procedure, cDNAs were ligated into the vector pCR-Topo2.1 (Invitrogen) and transformed into E. coli competent cells following the manufacturers recommendations. 1000 clones from each library were stored as glycerols in 96 well format.

Template Preparation and Library Sequencing

For sequencing template preparation PCR reactions were carried out in 384-well plates using the M13 forward (GTAAAACGACGGCCAG) and Reverse primers (CAGGAAACAGCTATGAC). The Biomek 2000 liquid handling robot was used to transfer 1 μl aliquots from each of 4×96-well plates containing overnight cultures into a conical bottomed 384-well plate (ABGen). PCR products were precipitated using 1 μl of 3M NaoAC (pH 6) and 15 μl of isopropanol and placed at −80° C. for at least one hour before centrifugation at 4K for 1 hr (4° C.). Pellets were washed with 20 μl of 70% ethanol and centrifuged for a further 30 min at 4K (4° C.) before they were air dried and resuspended in 10 μl of sterile MQ water. Products were checked by running 1 μl samples on a 1% agarose gel (1×TAE).

Sequencing reactions were performed in conical bottomed 384-well plates (Applied Biosystems) using the BigDye Terminator Cycle Sequencing Kit (Applied Biosystems). One μl of PCR product was added to 9 μl of sequencing mix (0.8 μl of 2 μM M13 Reverse primer; 0.5 μl Big Dye (Version 3); 3.5 μl ABI dilution buffer (400 mM Tris pH9; 10 mM MgCl₂) and 4.2 μl sterile MQ water) and the plate centrifuged briefly to collect the contents at the bottom of the wells. Cycle sequencing was performed using 40 cycles of 95° C. for 20 sec, 50° C. for 15 sec and 60° C. for 1.5 min (iCycler, Bio-Rad, USA). Sequencing products were precipitated by the addition of 1 μl of 3M NaOAc (pH 4.6), 1 μl sterile MQ water and 23 μl non-denatured 95% ethanol, placed on the bench for 15 min (at RT) and centrifuged at 4K for 30 min (4° C.). Immediately following centrifugation, plates were turned upside down on to several sheets of paper towels and centrifuged at 50×g for 1 min to expel all liquid. Any remaining liquid was removed by briefly spinning the plate in a salad spinner and the pellets resuspended in 10 μl of HiDi™ formamide solution (Applied Biosystems). Sequencing was performed on the ABI 3100 (Applied Biosystems) using a 36 cm array.

### EXAMPLE 6

### Construction of EST Database from in Vitro Cultures

To identify additional genes involved in the lolitrem biosynthetic pathway an approach of EST sequencing from N. lolii in vitro culture derived cDNA libraries was adopted. ESTs within the libraries derived from N. lolii and with homology to genes from the paxilline biosynthetic pathway are good candidates for orthologous lolitrem biosynthetic genes. It was expected that some genes may be expressed in in vitro cultures but many may only be expressed in planta so an in planta approach is described in example 5. The in vitro culture libraries were derived from liquid cultures in both rich and minimal media to increase the chance of identifying ESTs that may only be expressed under starvation conditions.

Culture Conditions

N. lolli strain Lp19 was initially cultured on potato dextrose agar plates. Mycelia from the leading edge of colonies were removed and chopped up finely with a scalpel blade before being transferred to 50 ml potato dextrose broth and incubated for 10 days at 25° C./200 rpm. Mycelia for RNA extraction were harvested under vacuum using a sterile buchner funnel containing two layers of Whatman 3 MM paper. Mycelia were washed three times with sterile MQ water. Dry weight was estimated from the wet weight.

To grow mycelia in minimal media, mycelia from N. lolli strain Lp19 cultures initially grown in complete medium for 14 days were harvested under vacuum using a sterile buchner funnel containing two layers of Whatman 3 MM paper. Mycelia were washed three times with sterile MQ water before transfer to the minimal medium. Two grams of mycelia was used to inoculate 50 ml of Blankenship MM and the cultures incubated for 19 days at 25° C./200 rpm. Mycelia for RNA extraction were harvested under vacuum using a sterile buchner funnel containing two layers of Whatman 3 MM paper. Mycelia were washed three times with sterile MQ water. Dry weight was estimated from the wet weight.

Isolation of Total RNA from Cultures Grown in Complete Medium

Mycelia were ground in liquid nitrogen using a sterile mortar and pestle and the RNA extracted using 1 ml Trizol® Reagent (Invitrogen) per 100 mg of ground mycelia. Samples were mixed well and frozen overnight at −20° C. The following day samples were thawed at RT on an orbital mixer (approx. 1 hr) and centrifuged at 12000×g for 10 min (4° C.) to remove polysaccharides. The supernatant was removed to a fresh tube and 0.2 ml of chloroform added per 1 ml Trizol reagent. Tubes were capped well and shaken vigorously by hand for 15 s and incubated at RT for 2 to 3min. Samples were centrifuged at 12 000×g for 15 min at 4° C. and the supernatant removed with a pipette to a fresh tube. RNA was precipitated using a modified precipitation step that effectively precipitated the RNA while maintaining polysaccharides and proteoglycans in a soluble form. Essentially, 0.25 ml isopropanol was added to the supernatant followed by 0.25 ml of a high salt precipitation solution (0.8M sodium citrate and 1.2M NaCl) per 1 ml of Trizol reagent used for the initial homogenization. The resulting solution was mixed well and the samples incubated at RT for 10 min. Samples were centrifuged at 12000×g for 10 min at 4° C. and the resulting RNA pellet washed once with 75% ethanol (1 ml 75% ethanol per 1 ml Trizol). The sample was mixed by vortexing and centrifuged at 7500×g for 5 min at 4° C.

The RNA pellet was briefly air dried for 5-10 min at RT and dissolved in 1 ml RNase free water (Invitrogen) with 1 μl Protector RNase (Roche) by passing the solution several times through a pipette tip and incubating for 10 min at 55-60° C. RNA purity and concentration were determined by spectrophotometry (A_{260/280}) and by running 3 μl and 6 μl aliquots (containing 1 μl of 10×MOPS running dye (0.2 M MOPS (pH7), 20 mM sodium acetate, 10 mM EDTA (pH8) in a total volume of 10 μl) on a 1% agarose gel in 1×TAE buffer containing ethidium bromide (1 μg/ml). RNA was stored as 10 μl aliquots at −80° C.

Isolation of Total RNA from Cultures Grown in Minimal Medium

Mycelia were ground in liquid nitrogen using a sterile mortar and pestle and the RNA extracted using 1 ml Trizol®) Reagent (Invitrogen) per 100 mg of ground mycelia. Samples were mixed well and frozen overnight at −20° C. The following day samples were thawed at RT on an orbital mixer (approx. 1 hr) and 0.2 ml chloroform added per 1 ml of Trizol reagent. Samples were vigorously shaken by hand for 15 s and incubated at RT for 2-3 min. Samples were centrifuged at 12 000×g for 15 min at 4° C. and the upper aqueous phase removed to a fresh tube. RNA was precipitated using 0.5 ml isopropanol per 1 ml Trizol reagent used for the initial homogenization. Samples were incubated at RT for 10 min and centrifuged at 12 000×g for 10 min at 4° C. The RNA pellet was washed using 1 ml 75% ethanol per 1 ml Trizol reagent used for the initial homogenization. Samples were mixed by vortexing and centrifuged at 7500×g for 5 min at 4° C. The RNA pellet was briefly air dried for 5-10 min at RT and dissolved in 1 ml RNase free water (Invitrogen) with 1 μl Protector RNase (Roche) by passing the solution several times through a pipette tip and incubating for 10 min at 55-60° C. RNA purity and concentration were determined by spectrophotometry (A_{260/280}) and by running 3 μl and 6 μl samples on a 1% agarose gel in 1×TAE buffer containing ethidium bromide. RNA was stored as 10 μl aliquots at −80° C.

Purification of mRNA

mRNA was purified from total RNA using the mRNA Purification Kit (Amersham Pharmacia Biotech) as per the manufacturer's instructions. Each Oligo (dT)-cellulose column had the capacity to bind approximately 25 μg of poly(A)⁺RNA so, assuming that only 2% of the total RNA was polyadenylated, no more than 1.25 mg of total RNA was applied to each column. mRNA was subjected to two rounds of purification and the concentration determined by spectrophotometry (A_{260/280}). Aliquots were stored at −80° C.

First-Strand cDNA Synthesis Using mRNA

Two μl of mRNA was combined with 1 μl SMART IV™ oligonucleotide and 1 μl CDS lll/3′ PCR primer in a sterile thin-walled 0.2 ml PCR tube (Bio-Rad). The contents were mixed, spun briefly in a microfuge and incubated at 72° C. for 2 min. The tube was then cooled on ice for 2 min, spun briefly to collect the contents at the bottom of the tube and the following added:

| | |
|---|---|
| 2 μl | 5X First-Strand Buffer |
| 1 μl | DTT (20 mM) |
| 1 μl | dNTP mix (10 mM) |
| 1 μl | PowerScript ™ Reverse Transcriptase |

Samples were mixed by gentle pipetting, briefly spun to collect the contents and incubated at 42° C. for 1 hr (Bio-Rad iCycler). The tube was placed on ice to terminate first-strand synthesis, 1 μl of sodium hydroxide (25 mM) added and the tube incubated at 68° C. for 30 min. A 3 μl aliquot was removed for cDNA amplification by Primer Extension PCR and the remaining first-strand cDNA stored at −20° C.

First-Strand cDNA Synthesis Using Total RNA

Three μl of freshly-prepared total RNA was combined with 1 μl SMART IV oligonucleotide and 1 μl CDS lll/3′ PCR primer in a sterile thin-walled 0.2 ml PCR tube (Bio-Rad). The contents were mixed, spun briefly in a microfuge and incubated at 72° C. for 2 min. The tube was then cooled on ice for 2 min, spun briefly to collect the contents at the bottom of the tube and the following added:

| | |
|---|---|
| 2 μl | 5X First-Strand Buffer |
| 1 μl | DTT (20 mM) |
| 1 μl | dNTP mix (10 mM) |
| 1 μl | PowerScript ™ Reverse Transcriptase |

Samples were mixed by gentle pipetting, briefly spun to collect the contents and incubated at 42° C. for 1 hr (Bio-Rad iCycler). The tube was placed on ice to terminate first-strand synthesis and a 3 μl aliquot removed for cDNA amplification by Long Distance (LD) PCR. The remaining first-strand cDNA was stored at −20° C.

cDNA Amplification by Primer Extension PCR

The following components were combined in a sterile 0.2 ml thin-walled PCR tube:

| | |
|---|---|
| 11 μl | First Strand cDNA (from section 3.1) |
| 71 μl | sterile MQ water |
| 10 μl | 10X Advantage 2 PCR buffer |
| 2 μl | 50X dNTP mix |
| 2 μl | 5′ PCR primer |
| 2 μl | CDS III/3′ PCR primer |
| 2 μl | 10X Advantage 2 Polymerase mix |

Samples were mixed, briefly spun to collect the contents and amplified by PCR (72° C. for 10 min, 95° C. for 20 s and 3cycles of 95° C. for 5 s, 68° C. for 8 min) using the Bio-Rad iCycler. A 10 μl sample was analysed on a 1.0% agarose gel (1×TAE) alongside 0.1 μg of a 1 kb plus DNA size marker (Invitrogen). The ds cDNA either underwent subsequent proteinase K and Sfi I digestions or was stored at −20° C. until further use.

cDNA Amplification by LD PCR

The following components were combined in a sterile 0.2 ml thin-walled PCR tube (Bio-Rad):

| | |
|---|---|
| 3 μl | First-Strand cDNA (from section 3.2) |
| 79 μl | sterile MQ water |
| 10 μl | Advantage 2 PCR buffer |
| 2 μl | 50X dNTP mix |
| 2 μl | 5′ PCR Primer |
| 2 μl | CDS III/3′ PCR Primer |
| 2 μl | 50X Advantage 2 Polymerase Mix |

Samples were mixed by gently flicking the tube, briefly spun to collect the contents and amplified by PCR (95° C. for 30 s and 26 cycles of 95° C. for 15 s, 68° C. for 6 min) using the Bio-Rad iCycler. The ds cDNA either underwent subsequent proteinase K and Sfi I digestions or was stored at −20° C. until further use.

Four μl of Proteinase K (20 μg/μl) and 5 μl of sterile MQ water were added to 90 μl of amplified ds cDNA, mixed and incubated at 45° C. for 20 min. The reaction was cleaned up using the Qiagen PCR Purification Kit as per the manufacturer's instructions and the cDNA eluted from the column in a total volume of 50 μl.

The following components were added to a fresh 0.2 ml thin-walled PCR tube:

| | |
|---|---|
| 50 μl | cDNA (proteinase K treated) |
| 29 μl | sterile MQ water |
| 10 μl | 10X Sfi I buffer |
| 10 μl | Sfi I restriction enzyme |
| 1 μl | 100X BSA |

Samples were mixed well and incubated at 50° C. for 2 hr.

Following Sfi I digestion, 2 μl of a 1% xylene cyanol solution was added to the tube and the sample mixed well. Sixteen sterile 1.5 ml tubes were labelled and arranged in a rack in order. A CHROMA SPIN-400 column (Clontech) was prepared as per the manufacturer's instructions and the mixture of Sfi I-digested cDNA and xylene cyanol dye carefully applied to the top centre surface of the column matrix. Once the sample was fully absorbed into the matrix, 100 μl of column buffer was also applied to the column and the buffer allowed to drain from the column until there was no liquid remaining above the resin. At this point, the dye layer was several mm into the column.

The rack containing the 1.5 ml collection tubes was placed so that the first tube was directly underneath the column outlet. 600 μl of column buffer was added to the column and single-drop fractions (approximately 35 μl per tube) collected in the labelled tubes. The profile of each fraction was checked by analysing 10 μl samples alongside 0.1 μg of a 1 kb plus DNA standard (Invitrogen) on a 1.1% agarose gel (1×TAE; 150V; 10 min). The gel was stained with ethidium bromide for 15 min, destained in water for 1.5 hr and the peak fractions determined by visualizing the intensity of the bands under UV. The first 3 fractions containing cDNA were collected and pooled. Samples were cleaned up using an Amicon-30 unit (Millipore). The unit was washed twice with sterile MQ water before use as per the manufacturer's instructions. The pooled fractions were applied to the unit and concentrated to 7 μl by centrifugation at 14 000 g for 20 min at room temperature. The Sfi I-digested cDNA was either stored at −20° C. or used immediately in the ligation reaction.

Ligation of Sfi I-Digested cDNA to the A TriplEx2 Vector and Library Packaging

Ligations were optimized using three different ratios of cDNA to phage vector following the manufacturers recommendations. Samples were mixed gently, centrifuged briefly to bring the contents to the bottom of the tube and incubated overnight at 16° C. Ligations (cDNA/ATriplEλ2 Vector) were heat inactivated at 65° C. for 15 min. Packaging reactions (50 μl) were thawed at room temperature and placed on ice. Half of the packaging extract (25 μl) was immediately transferred to a second ice-cold 1.5 ml tube. The entire ligation (7 μl) was added to 25 μl of packaging extract, mixed gently with a pipette and incubated at 30° C. for 90 min. At the end of this incubation, the remaining 25 μl of packaging extract was added to the sample and the reaction incubated for a further 90 min at 30° C. Five hundred μl of 1×Lambda dilution buffer (100 mM NaCl, 10 mM MgSO₄.7H₂O, 35 mM Tris-HCl (pH 7.5), 0.01% gelatin) was added to the sample and mixed by gentle vortexing. Chloroform (25 μl) was also added to prevent bacterial contamination. Packaged libraries were titered following the manufacturers recommendations and stored at 4° C. for up to one month.

Library Amplification

A single, well-isolated colony of XL1-Blue was picked from the primary working plate and used to inoculate 15 ml of LB broth containing MgSO₄ (10 mM) and maltose (0.2%). Cultures were incubated at 37° C. overnight with shaking (140 rpm). Cells were harvested the following day by centrifuging the culture at 5K for 5 min. The supernatant was removed by decanting and the pellet resuspended in 7.5 ml of 10 mM MgSO₄. Enough phage to yield 6-7×10⁴ plaques per 150 mm plate was added to each of 10 tubes containing 500 μl of overnight XL1-Blue culture in a sterile 1.5 ml tube. Phage were allowed to adsorb to the E. coli cells by incubating in a 37° C. water bath for 15 min before adding 4.5 ml of melted (45° C.) LB top agar containing MgSO₄ (10 mM) and maltose (0.2%). Samples were quickly mixed by gentle vortexing and immediately poured on to prewarmed (37° C.) 150 mm LB agar plates containing MgSO₄(10 mM). Plates were cooled for 10 min at room temperature to allow the top agar to harden and incubated at 37° C. for 10.5 hr. Phage were eluted by adding 12.5 ml of 1×Lambda dilution buffer to each plate and the plates stored overnight at 4° C. The following day, the plates were shaken (˜50 rpm) at room temperature for 1 hr and the phage lysates poured into a sterile beaker. Intact cells were lysed by adding 10 ml of chloroform and the phage lysate cleared of cell debris by centrifuging at 5000×g for 10 min in sterile 50 ml polypropylene tubes. The supernatant was collected and stored at 4° C. in sterile universals. For long-term storage, 1 ml aliquots were made containing DMSO to a final concentration of 7% and frozen at −80° C.

Converting A TriplEx2 to pTriplEx

The bacterial host strain E. coli BM25.8 (supE44, thi Δ(lac-proAB) relA1, [F′lacl^{q}ZΔM15, proAB⁺, traD36], hsdR(rₖ₁₂−mₖ₁₂−), (kan^{R})P1 (cam^{R}) λimm434) was supplied as a component of the SMART cDNA Library Construction Kit (Clontech) and stored at −80° C. For large-scale library conversion a single, well-isolated colony of E. coli BM25.8 was picked from the primary working plate and used to inoculate 10 ml of LB broth. Cultures were incubated at 31° C. overnight with shaking (150 rpm). The following day, MgCl₂ (10 mM) was added to the overnight culture of BM25.8. In a sterile 15 ml tube, 200 μl of overnight culture was mixed with 2×10⁶ pfu/ml of amplified λTriplEx2 cDNA library and incubated for 1 hr at 31° C. (without shaking). After the incubation was complete, 500 μl of LB broth was added and the sample incubated for a further 1 hr at 31° C. with shaking (190 rpm). At this point, conversion of the library to plasmid form was complete. The converted cDNA library was diluted 1:100 in LB broth and aliquots (10 μl, 100 μl) were spread on to LB agar plates containing carbenicillin (50 μg/ml). Plates were incubated overnight at 31° C. and the colonies picked for further analysis. The remaining converted library was stored as 1 ml aliquots containing glycerol (to a final concentration of 30%) at −80° C.

PCR Analysis

Individual colonies from converted libraries were inoculated into 100 μl of LB broth containing carbenicillin (50 μg/ml) in round bottomed 96-well plates (Nunc). Plates were incubated overnight at 37° C. Aliquots of 1 μl of each overnight culture were PCR amplified in a total volume of 15 μl using ptriplex2FORWARD (5′-MGCGCGCCATTGTGTTGGTACCC-3′) and ptriplex2REVERSE (5′-CGGCCGCATGCATMGCTTGCTCG-3′) as primers (present in the pTriplEx vector arms) (Kohler et al., 2003). The PCR included 95° C. for 3 min, 95° C. for 60 s, 60° C. for 30 s, 72° C. for 3 min for 30 cycles and a final extension of 72° C. for 15 min (iCycler, Bio-Rad, USA). One μl of each reaction was analysed on a 1% agarose gel alongside 0.25 μg of a 1 kb plus DNA standard (Invitrogen) and stained with ethidium bromide to determine the size and quality of the PCR products.

For sequencing template preparation PCR reactions were carried out in 384-well plates. The Biomek 2000 liquid handling robot was used to transfer 1 μl aliquots from each of 4 x 96-well plates containing overnight cultures into a conical bottomed 384-well plate (ABGen). PCR products were precipitated using 1 μl of 3M NaoAC (pH 6) and 15 μl of isopropanol and placed at −80° C. for at least one hour before centrifugation at 4K for 1 hr (4° C.). Pellets were washed with 20 μl of 70% ethanol and centrifuged for a further 30 min at 4K (4° C.) before they were air dried and resuspended in 10 μl of sterile MQ water. Products were checked by running 1 μl samples on a 1% agarose gel (1×TAE) and further diluted either 1:5 (minimal medium cDNA library) or 1:1 (complete medium cDNA library) in sterile MQ water before sequencing.

Sequencing Reactions

Sequencing reactions were performed in conical bottomed 384-well plates (Applied Biosystems) using the BigDye Terminator Cycle Sequencing Kit (Applied Biosystems). One μl of PCR product was added to 9 μl of sequencing mix (0.8 μl of 2 μM ptriplex2FORWARD primer; 0.5 μl Big Dye (Version 3); 3.5 μl ABI dilution buffer (400 mM Tris pH9; 10 mM MgCl₂) and 4.2 μl sterile MQ water) and the plate centrifuged briefly to collect the contents at the bottom of the wells. Cycle sequencing was performed using 40 cycles of 95° C. for 20 sec, 50° C. for 15 sec and 60° C. for 1.5 min (iCycler, Bio-Rad, USA). Sequencing products were precipitated by the addition of 1 μl of 3M NaOAc (pH 4.6), 1 μl sterile MQ water and 23 μl non-denatured 95% ethanol, placed on the bench for 15 min (at RT) and centrifuged at 4K for 30 min (4° C.). Immediately following centrifugation, plates were turned upside down on to several sheets of paper towels and centrifuged at 50×g for 1 min to expel all liquid. Any remaining liquid was removed by briefly spinning the plate in a salad spinner and the pellets resuspended in 10 μl of HiDi™ formamide solution (Applied Biosystems). Sequencing was performed on the ABI 3730 (Applied Biosystems) using a 50 cm array.

### EXAMPLE 7

### Identification of ESTs Encoding Putative Lolitrem Biosynthetic Genes from EST Sequence Databases

A sequence database was developed containing the 4000 EST sequences derived from the SSH libraries and 6500 ESTs derived from in vitro culture libraries. The database was searched using the BLAST algorithm. Nucleotide sequences were blasted using the BlastX algorithm against the SwissProt database. ESTs with homology to paxilline biosynthetic genes are listed in Table 6. All paxilline orthologs were identified in the Nle+M library.

TABLE 6
Detail of ESTs with Homology to Paxilline Biosynthetic Genes

| | Length | | Paxilline | Blast |
|---|---|---|---|---|
| EST | (bp) | Function | Homolog | Score |
| E07 | 353 | dimethylallyltryptophan | paxD | 5e−02 |
| | | (DMAT) synthase | | |
| DMAT | 532 | dimethylallyltryptophan | paxD | |
| Johnson1 | | (DMAT) synthase | | |
| N17 | 413 | Cytochrome P450 | — | 2e−09 |
| | | monooxygenase | | |
| G13 | 335 | Cytochrome P450 | paxP | 3e−07 |
| | | monooxygenase | | |
| J15 | 639 | Cytochrome P450 | paxP | 8e−34 |
| | | monooxygenase | | |

### EXAMPLE 8

### Predicted Genes in Lolitrem Gene Cluster and Isolation of Two Additional ltm Gene Clusters

Isolation of Lolitrem Biosynthetic Genes

We describe in examples 2 and 3 the molecular cloning and genetic analysis of a set of genes from N. lolii and E. festucae that are proposed to be involved in the biosynthesis of lolitrem and closely related indole-diterpenes. This is the second indole-diterpene gene cluster to be cloned from a filamentous fungus. We recently reported on the isolation of a cluster of genes from P. paxilli required for paxilline biosynthesis (Young et al. 2001). A comparison with the paxilline biosynthesis cluster identifies five functional orthologues, ltmG, ltmM, ltmP, ltmQ and ltmD. In addition we have identified two P450 genes, ltmJ and ltmK, that may also play a role in indole-diterpene biosynthesis in this group of fungi. Three of these genes, ltmG, ltmM and ltmK form a tightly linked cluster.

The first of these genes, ltmG, is clearly identifiable as a GGPP synthase, and is presumed to catalyse the first step in the biosynthesis of lolitrems i.e. the synthesis of GGPP. Interestingly, the two fungal species in which diterpene gene clusters have been analysed, have two copies of GGPP synthase, one proposed to be involved in primary metabolism and one specifically recruited for secondary metabolism (Tudzynski and Hölter 1998; Young et al. 2001). N. lolii and E. festucae also have two copies of a GGPP synthase. One copy is proposed to be required for primary metabolism and the second copy (ltmG) is proposed to be specifically required for indole-diterpene biosynthesis.

Deletions of paxM and paxC in P. paxilli result in mutants with a paxilline-negative phenotype. To date no identifiable indole-diterpene intermediates have been identified in these strains, suggesting that these genes are involved in very early steps in the pathway. Our working model is that PaxM and PaxC are required to catalyse the epoxidation and cyclisation of GGPP and addition of indole-3-glycerol to form the first stable indole-diterpene, possibly paspaline (Parker and Scott 2004). By analogy we propose that LtmM catalyses the same early reaction in lolitrem biosynthesis. In support of this hypothesis we were able to demonstrate that ltmM is required for lolitrem biosynthesis by making a targeted deletion of this gene. Mutants deleted in this gene were unable to synthesize lolitrem B in artificial symbiota with perennial ryegrass. An N. lolii orthologue of paxC, is yet to be identified, but is predicted to also be essential for lolitrem biosynthesis.

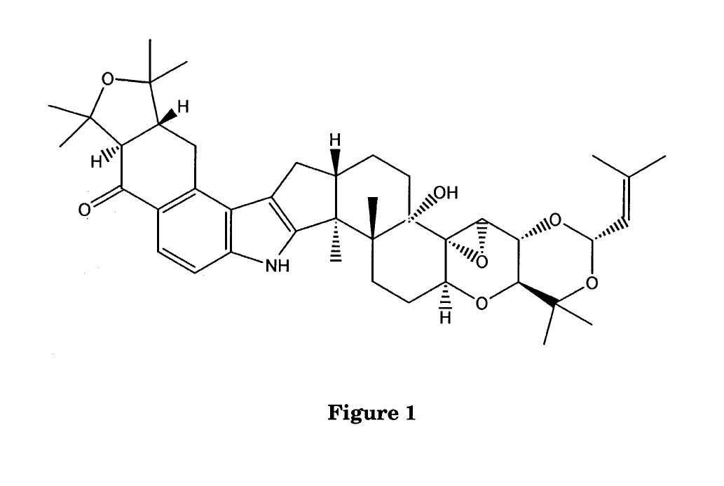

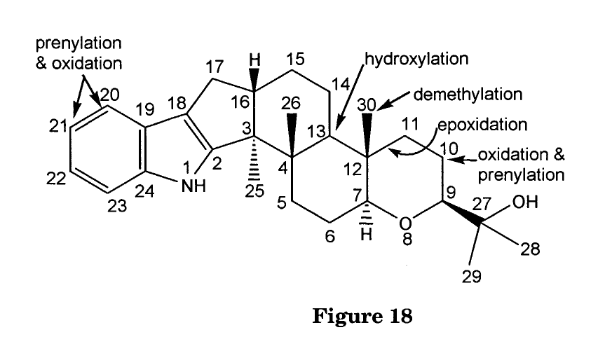

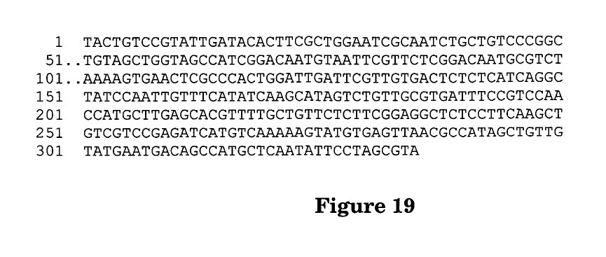

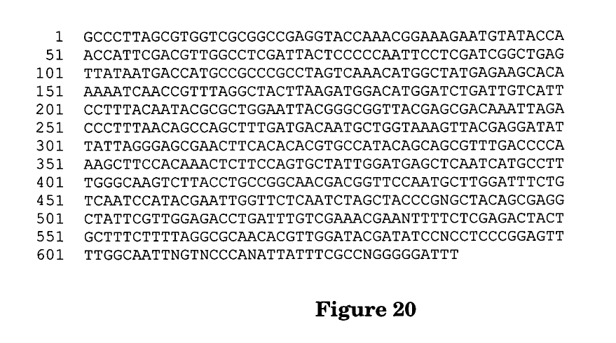

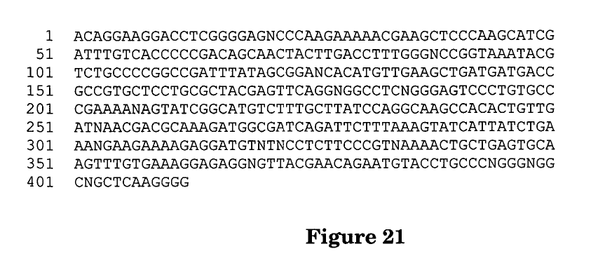

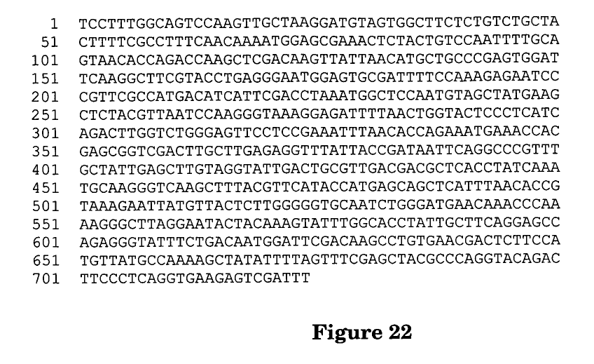

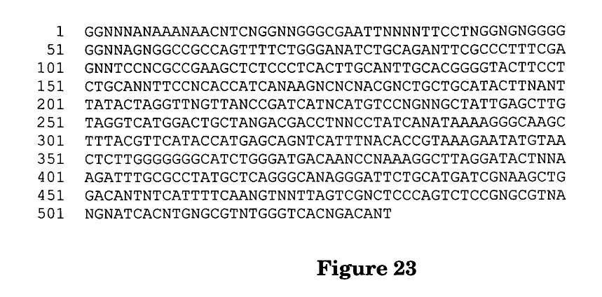

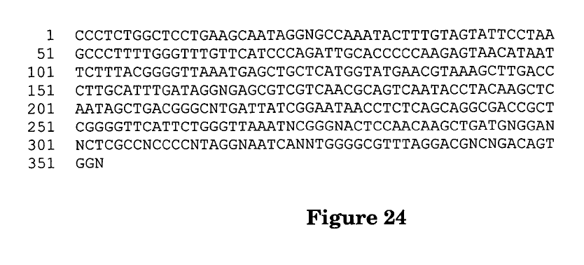

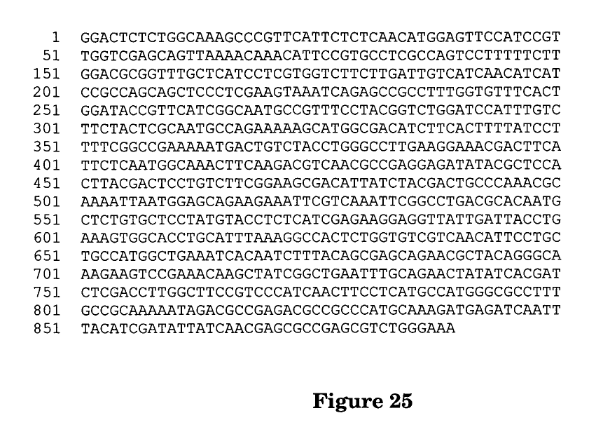

Other genes identified as being necessary for paxilline biosynthesis are paxP and paxQ; which encode cytochrome P450 enzymes. Targeted deletion of paxP and paxQ results in strains that accumulate paspaline and 13-desoxypaxilline, respectively (McMillan et al. 2003). These results suggest that PaxP is required for demethylation of C-12 of paspaline, and possibly hydroxylation of C-10, and PaxQ is required for hydroxylation of C-13, using either PC-M6 or 13-desoxypaxilline as substrates (FIG. 18). Analysis of the structure of lolitrem B (FIG. 1) suggests that similar modifications are required to the paspaline skeleton (FIG. 18) to generate lolitrem B. Orthologues of paxP and paxQ were identified in an EST library generated with template from suppression subtractive hybridization. A further enzyme predicted to be required for lolitrem B biosynthesis is a prenyl transferase to prenylate positions 20 and 21 of the indole ring. A candidate gene for one or both of these prenylations is ltmD, given that the paralogue, dmaW, prenylates position 20, as the first committed step in ergot alkaloid biosynthesis (Wang et al. 2004). One or two additional cytochrome P450 enzymes are predicted to be required for further oxidation and closure of ring A of lolitrem B. Candidates for these functions include ltmJ and ltmK. At least two additional enzymes are required to form an epoxide between C-11 and C-12 of paspaline, and prenylate ring H of lolitrem B. These would be predicted to be an FAD-dependent monooxygenase and a prenyl transferase, respectively. We have yet to identify these genes.

In summary, we predict that up to ten genes are required for the biosynthesis of lolitrem B. Candidate genes identified to date include ltmG, ltmM, ltmK, ltmP, ltmQ, ltmD and ltmJ. Deletion analysis has confirmed that at least ltmM is required for lolitrem B biosynthesis. Further genetic analysis of the genes identified here and adjacent genes will help elucidate the pathway for lolitrem biosynthesis. A comparison with the steps required for paxilline biosynthesis in P. paxilli will elucidate the basic biochemistry and genetics of this important group of secondary metabolites. In Example 7 we describe the isolation of ESTs that may correspond to ltmP, ltmD and ltmJ. In this example we describe a method for isolation of genomics regions containing the additional predicted ltm genes.

Isolation of Additional ltm Genes

The presence of the retrotransposon platforms and the instability of cloned fragments of the regions directly flanking the ltmG, ltmM and ltmK cluster made it difficult to isolate additional flanking sequences. Therefore we used EST sequences with homology to the paxP gene to isolate an orthologue (ltmP). The EST sequences G13, J15 and N17 isolated from endophyte infected ryegrass in Example 7 showed significant homology to paxP (Table 6). In this example we demonstrated that these ESTs corresponded to ltmP and were linked to the cluster containing ltmG, ltmM and ltmK.

The EST sequences G13, J15 and N17 did not align to the cluster containing ltmG, ltmM and ltmK suggesting they were unique. Primers were then designed to regions that were highly conserved to paxP with a consideration on the placement of possible conserved introns between the ltm and pax genes.

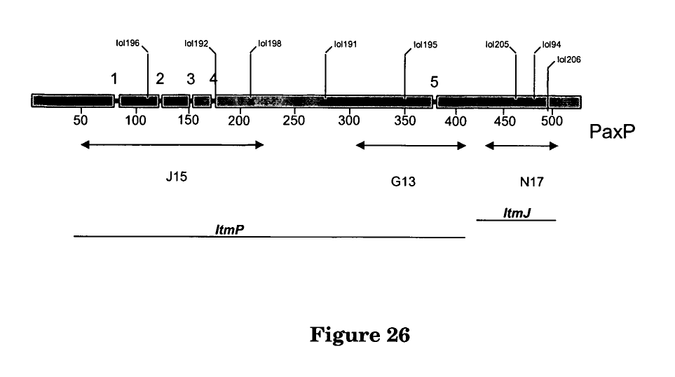

EST sequences with BLASTX matches to paxP aligned into three independent contigs (FIG. 26). Contig 1 contained EST sequence J15, contig 2 contained EST sequences G13, and contig 3 contained EST sequence N17. PCR was performed to test whether these three contigs were part of a single cytochrome P450 monooxygenase gene or were in fact multiple genes. Amplification of LP19 genomic DNA with primers lol192 and lol195 linked contigs 1 and 2 and therefore these two contigs are apart of the same fungal cytochrome P450 monooxygenase gene subsequently named ltmP. The PCR fragment generated from LP19 genomic DNA with primers lol192 and lol195 was sequenced and compared to the EST data for confirmation of the intron. Contig 3 contained the primer binding site for primer lol194 and this primer would not amplify a PCR product from LP19 genomic DNA when paired with primer lol192. This contig was therefore considered an independent cytochrome P450 monooxygenase fragment and was subsequently named ltmJ. Primers, lol205 and lol206, were designed to the contig sequence of ltmJ. These primers amplified a 242 bp fragment from Lp19 genomic DNA and confirmed that ltmJ was of fungal origin.

TABLE 9
Primers Used un this example and not
listed in table 2

| Primer | | |
|---|---|---|
| name | Sequence 5′→3′ | Used for |
| lol191 | CCAAAGGAGGTTTTGAATGTA | ltmP PCR/probe |
| lol192 | TTGGATGAGCTCAATCATGC | ltmP PCR/probe/RT-PCR |
| lol194 | GAACTCGTAGCGCAGGAGCA | ltmJ PCR |
| lol195 | TTCTCTTCGGAGGCTCTCCT | ltmP PCR |
| lol196 | TGGACATGGATCTGATTGTC | ltmP probe |
| lol198 | TGTAGCACGGGTAGCTAGAT | ltmP probe |
| lol199 | TTGCGCATCGTACGCTAGGA | IPCR |
| lol202 | GGATGAAGAAAATCCACGAG | IPCR |
| lol203 | AGACGATCTGTTAGGCCGAT | IPCR |
| lol205 | CCAAGCATCGATTTGTCACC | ltmJ PCR/probe |
| lol206 | AATCTGATCGCCATCTTTGC | ltmJ PCR/probe |
| lol209 | GAATAGCTCAAGACTCAGAA | IPCR |
| lol210 | AAGCTGGCTGTTAAAGGGTC | IPCR |
| lol211 | TATTAGGGAGCGAACTTCAC | IPCR |
| lol213 | AAGAGGGCCGCAATTTCGAT | IPCR |
| lol222 | GCGTGCAACATTAACATTCTC | IPCR |
| lol235 | ATTCCACCATGGCATCTGGAG | ltmC complenetation |
| | CATGGCTCG | |
| lol236 | CTTAAGCGAATTCTACCTTGT | ltmC |
| | GGGTG | probe/complementation |
| lol341 | TTCCGCTTCCGAGTAGACTC | ltmE PCR/RT-PCR/probe |
| lol356 | CCGAGTTTGATGACCTGCTG | ltmE PCR/RT-PCR/probe |
| SP6 | CCATTTAGGTGACACTATAG | Seq |
| T1.1 | GAGAAAATGCGTGAGATTGT | tub2 probe/RT-PCR |
| T1.2 | CTGGTCAACCAGCTCAGGAC | tub2 probe/RT-PCR |

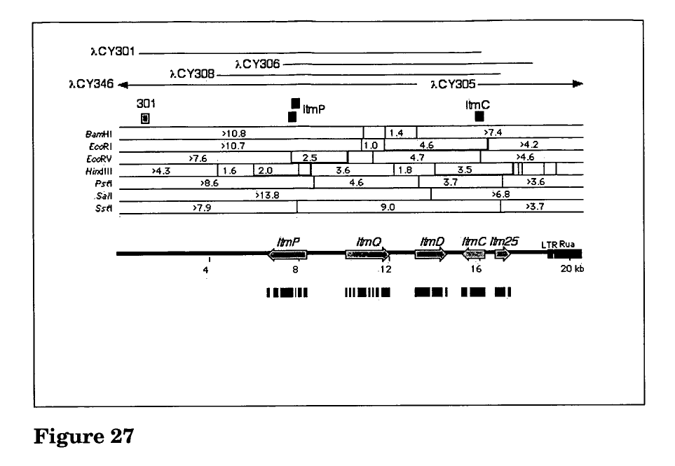

The ltmJ fragment hybridised to the lolitrem producing strains Lp19 and Fl1 (FIG. 27). This fragment hybridised to a ˜18 kb LP19 Sstl fragment, a band of the same size as seen with the ltmP probes suggesting linkage of ltmJ to ltmP. The presence of the three EST fragments, ltmC, ltmP and ltmJ, correlated with strains known to produce indole-diterpenes. None of the fragments hybridised to genomic digests of E8, a lolitrem non-producing strain. This pattern of hybridisation was used to identify the previous ltm cluster containing ltmG, ltmM and ltmK and therefore complete sequence surrounding the genes ltmC, ltmP and ltmJ was obtained.

ltm Cluster 2

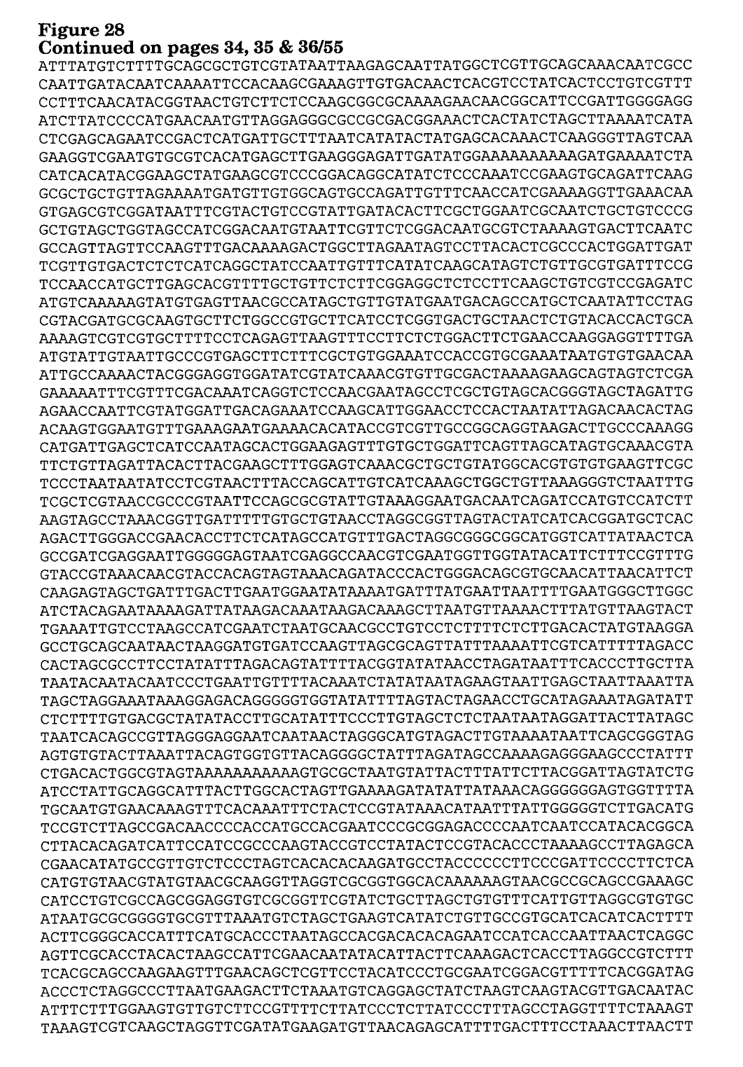

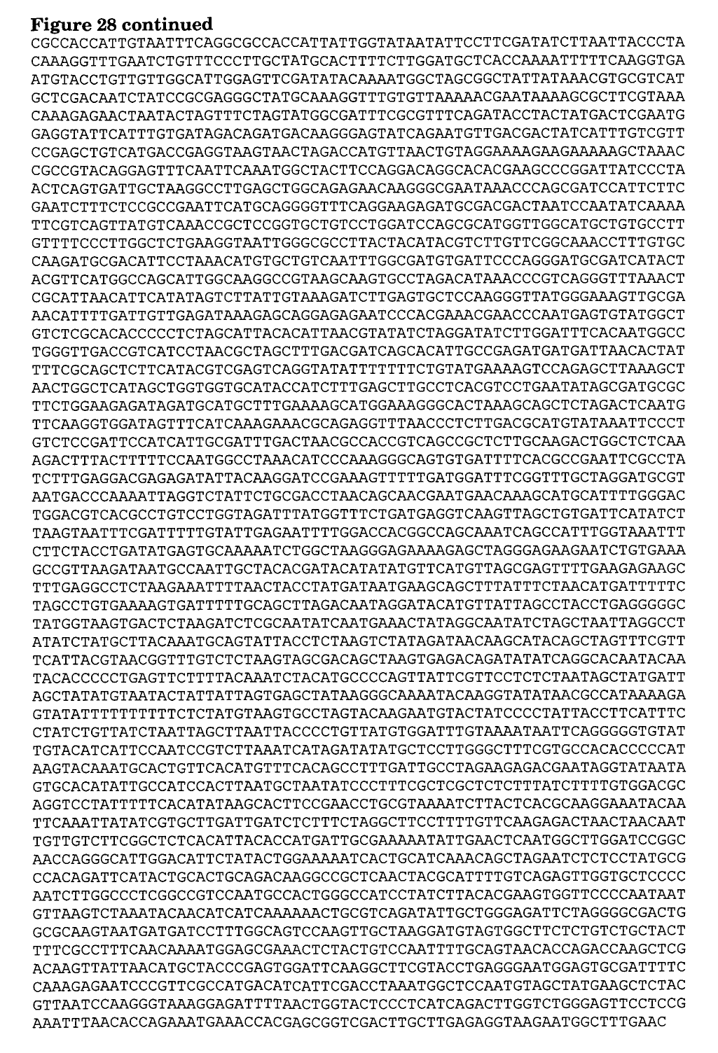

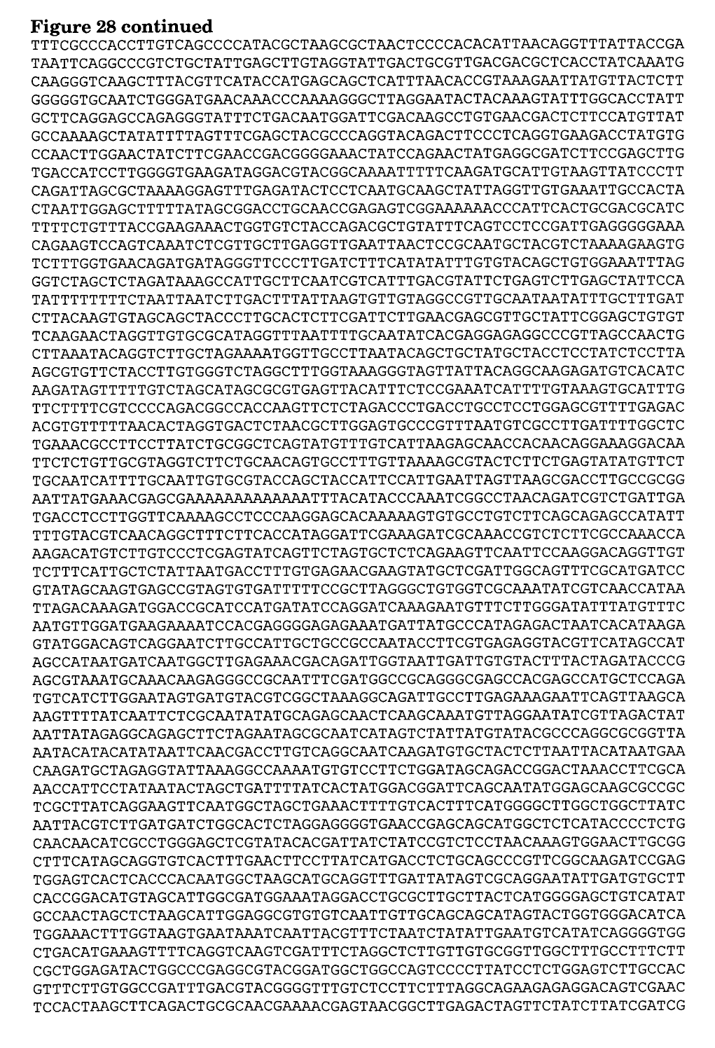

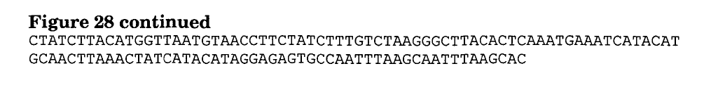

Initially the complete ltmC and ltmP genes were sequenced from LP19 using fragments generated by IPCR with the restriction enzymes EcoRI, EcoRV and Hindlll (FIG. 28). The complete ltmC gene was amplified using IPCR with LP19 Hindlll digested then self-ligated genomic DNA and primers, lol202 and lol203, that were designed to the previously obtained ltmC sequence. The sequence was extended using IPCR with LP19 EcoRI digested then self-ligated genomic DNA and primers, lol213 and lol209. The sequence of the complete ltmP gene was generated using IPCR with Lp19 Hindlll digested then self-ligated genomic DNA using two primer sets of lol198 and lol199, and lol210 and lol211. The sequence was extended further by IPCR using LP19 EcoRV digested then self-ligated genomic DNA with primers lol192 and lol222. Each IPCR fragment was cloned into pGEM-T easy (Promega) and sequenced with primers that were ltmC or ltmP sequence specific or with primers Sp6 and T7.

A LP19 λGEM-12 genomic library was screened with ltmP. LP19 λGEM-12 genomic library filters were screened with the ltmP fragment, amplified with lol191 and lol192, which resulted in the isolation of 25 positive clones. The average insert size of the 35-lambda clones was approximately 13 kb. DNA isolated from the lambda clones was digested with the restriction enzymes BamHl, EcoRI, Hindlll, and Sstl then hybridised with the ltmP fragment to determine clones for sequencing. Lambda clones of interest were sequenced with primers Sp6 and T7 that anneal to the lambda arms and then with sequence specific primers. To facilitate sequencing, fragments from some lambda clones were cloned into the pUC118 vector and sequenced with the forward and reverse primers. A physical map of the overlapping lambda clones (FIG. 27) was determined based on DNA sequence analysis and the hybridisation data from both the lambda clones and the genomic DNA (FIG. 27). Data generated from the physical map of the lambda clones showed that the following; λCY300, λCY307, λCY312, λCY313, λCY315, λCY316, λCY319 and λCY350, contained sequences that were inconsistent with the LP19 genomic map and therefore these sequences were not analysed further.

Nucleotide sequence generated from sequencing lambda clones isolated from the hybridisation with ltmP covered 23.8 kb. Sequence analysis of this region using BLAST algorithms identified three additional genes with significant similarities to pax genes from P. paxilli and therefore formed a second gene cluster called ltm cluster 2 (FIG. 27). The nucleotide sequence of N. lolii ltm cluster 2 is shown in FIG. 28. These genes included, ltmC, ltmP, ltmQ, ltmD, and ltm25, orthologues of paxC, a prenyl transferase, paxP and paxQ, cytochrome P450 monooxygenases, paxD, a prenyl transferase (a dimethylallyl tryptophan synthase-like) and sec25, a gene of unknown function, respectively (FIG. 27). The ltmJ gene was not contained within this sequenced region. The individual sequence analysis of the five genes contained in ltm cluster 2 is explained below and in Tables 7 and 8.

TABLE 3.11
The Itm genes from clusters 2 and 3, intron analysis and comparisons to database sequences

| | | | | | Intron | | | | Top | | | |
|---|---|---|---|---|---|---|---|---|---|---|---|---|
| | | | | | | | | 5′ . . . 3′ Splice | Database | | E | |
| Gene | Putative function | Cluster | size (aa) | kDa | No. | phase | size | sites | match¹ | Species | value | Reference |
| Itm25 | Unknown | 2 | 221 | 24.4 | 1 | 2 | 76 | GTAAGT . . . CAG | sec25² | P. paxilli | 2e-59 | |
| ItmC | Prenyl | 2 | 345 | 39 | 1 | 2 | 77 | GTATGT . . . TAG | paxC | P. paxilli | 1e-59 | Young et |
| | transferase | | | | | | | | | | | al 2001 |
| ItmD | Prenyl | 2 | 439 | 49 | 1 | 2 | 74 | GTAAGA . . . CAG | AN8514 | A. nidulans | 1e-46 | |
| | transferase | | | | | | | | | | | |
| | (dmaW-like) | | | | 1 | 2 | 102 | GTAAGT . . . TAG | | | | |
| ItmQ | P450 | 2 | 537 | 61.4 | 1 | 0 | 59 | GTTTGA . . . AAG | paxQ | P. paxilli | 1e-105 | Young et |
| | monooxygenase | | | | 2 | 0 | 61 | GTTTGT . . . TAG | | | | al 2001 |
| | | | | | 3 | 0 | 59 | GTAAGT . . . CAG | | | | |
| | | | | | 4 | 2 | 60 | GTAATGC . . . TAG | | | | |
| | | | | | 5 | 0 | 52 | GTATGG . . . TAG | | | | |
| | | | | | 6 | 0 | 53 | GTATAT . . . TAG | | | | |
| | | | | | 7 | 1 | 56 | GTATAA . . . CAG | | | | |
| ItmP | P450 | 2 | 498 | 57 | 1 | 0 | 59 | GTGTTC . . . CAG | paxP | P. paxilli | 1e-102 | Young et |
| | monooxygenase | | | | 2 | 1 | 49 | GTAAGT . . . CAG | | | | al 2001 |
| | | | | | 3 | 1 | 60 | GTATGT . . . GAG | | | | |
| | | | | | 4 | 1 | 57 | GTAAGG . . . AAG | | | | |
| | | | | | 5 | 0 | 107 | GTATGT . . . TAG | | | | |
| ItmJ | P450 | 3 | 525 | 60.4 | 1 | 0 | 63 | GTGAGC . . . CAG | AN1598 | A. nidulans | 4e-81 | |
| | monooxygenase | | | | 2 | 0 | 67 | GTGAGG . . . TAG | | | | |
| | | | | | 3 | 0 | 49 | GTAAGG . . . AAG | | | | |
| | | | | | 4 | 1 | 69 | GTATGT . . . TAG | | | | |
| | | | | | 5 | 1 | 61 | GTAAGA . . . CAG | | | | |
| | | | | | 6 | 1 | 58 | GTAAGT . . . TAG | | | | |
| ItmE | Prenyl | 3 | 788 | 87.5 | 1 | 2 | 67 | GTACGT . . . AAG | paxC³ | P. paxilli | 3e-60 | Young et |
| | transferase | | | | | | | | | | | al 2001 |
| | fusion | | | | | | | | | | | |
| | (paxC-paxD) | | | | 2 | 2 | 56 | GTGAGT . . . CAG | AN8514³ | A. nidulans | 7e-56 | |
| | | | | | 3 | 2 | 54 | GTACGT . . . TAG | | | | |
| chsV | Chitin synthase | | 1861 | 206 | 1 | | 82 | GTAGGT . . . TAG | AAF04279 | Blumeria | 0.0 | |
| | (class V) | | | | 2 | | 69 | GTAAGT . . . CAG | | graminis | | |

¹If the top match was to a pax gene, the gene name was used otherwise the gene is indicated by the accession or gene number.
²The top match was determined using the tBLASTn algorithm as the sec25 gene is currently not annotated
³This gene is a hybrid containing a paxC-like domain and a paxD-domain

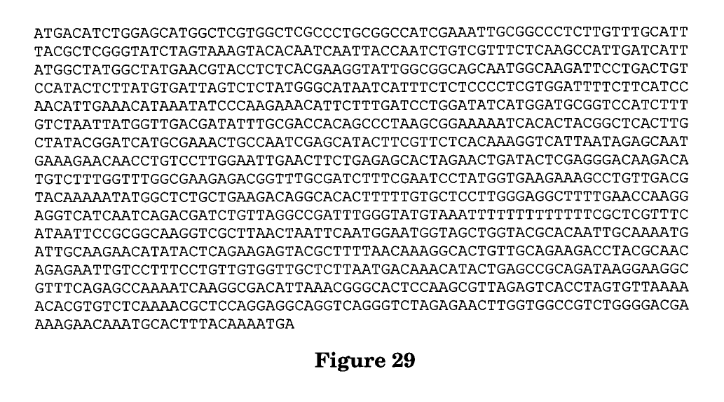

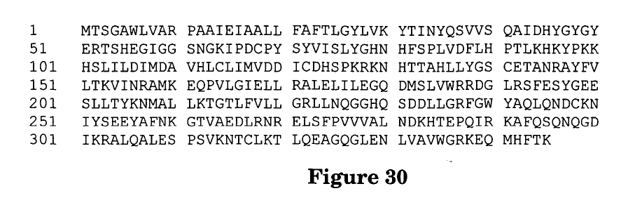

The nucleotide sequence analysis of the complete ltmC gene showed that it contains one intron (FIGS. 27 and 29) and encodes a polypepetide of 345 amino acids. LtmC is classified as a prenyl transferase as it contains the five conserved domains found in other prenyl transferases (Chen et al, 1994). FastA analysis showed that LtmC was more similar to AtmC from A. flavus than PaxC from P. paxilli. The single intron found in ltmC was conserved with placement and phase with the second of the two conserved introns found in P. paxilli paxC (Young et al, 2001) and A. flavus atmC (Zhang et al, 2004).

TABLE 3.12
Sequence identity of the ltm genes to their pax and atm homologues

| gene | Homologue | Species | % Identity | E-value | Analysis programme |
|---|---|---|---|---|---|
| ItmG | ggs1 | P. paxilli | 54.1 | 1e-90 | FASTA/BLASTP |
| | paxG | P. paxilli | 52.6 | | FASTA |
| | paxC | P. paxilli | 31.5 | | FASTA |
| | paxD | P. paxilli | 22.2 | | FASTA |
| | atmG | A. flavus | 59.4 | e-101 | FASTA/BLASTP |
| | atmC | A. flavus | 30.1 | | FASTA |
| | ItmC | N. lolii | 28.4 | | FASTA |
| | ItmD | N. lolii | 28.4 | | FASTA |
| | ItmE | N. lolii | 31.5 | | FASTA |
| ItmM | paxM | P. paxilli | 41 | 7e-96 | BLASTP |
| | atmM | A. flavus | 42.2 | e-100 | BLASTP |
| ItmK | paxP | P. paxilli | 31.3 | 7e-63 | FASTA/BLASTP |
| | paxQ | P. paxilli | 23.4 | | FASTA |
| | ItmJ | N. lolii | 36.8 | | FASTA |
| | ItmP | N. lolii | 28.6 | | FASTA |
| | ItmQ | N. lolii | 25.3 | | FASTA |
| Itm25 | sec25 | P. paxilli | 53.8 | 2e-59 | tBLASTn |
| | FG04594 | Fusarium graminearum | | 4e-46 | BLASTP |
| ItmC | paxC | P. paxilli | 43.3 | 1e-59 | FASTA/BLASTP |
| | paxG | P. paxilli | 28.4 | | FASTA |
| | atmC | A. flavus | 47.7 | 2e-68 | FASTA/BLASTP |
| | atmG | A. flavus | 28.1 | | FASTA |
| | ItmE | N. lolii | 55.8 | | FASTA |
| | ItmG | N. lolii | 28.4 | | FASTA |
| ItmD | paxD | P. paxilli | 24.2 | | FASTA |
| | ItmE | N. lolii | 37.1 | | FASTA |
| | ItmG | N. lolii | 24.2 | | FASTA |
| | dmaW | Neotyphodium LpTG-2 | 22.5 | | FASTA |
| | AN8514 | Aspergillus nidulans | | 1e-46 | BLASTP |
| ItmP | paxP | P. paxilli | 41.3 | e-102 | FASTA/BLASTP |
| | paxQ | P. paxilli | 24.4 | | FASTA |
| | ItmJ | N. lolii | 25 | | FASTA |
| | ItmK | N. lolii | 29.2 | | FASTA |
| | ItmQ | N. lolii | 24.5 | | FASTA |
| ItmQ | paxQ | P. paxilli | 38.1 | e-105 | FASTA/BLASTP |
| | paxP | P. paxilli | 28.7 | | FASTA |
| | ItmJ | N. lolii | 22.2 | | FASTA |
| | ItmK | N. lolii | 25.3 | | FASTA |
| | ItmP | N. lolii | 24.1 | | FASTA |
| ItmJ | paxP | P. paxilli | 29.2 | 1e-49 | FASTA/BLASTP |
| | paxQ | P. paxilli | 23.7 | | FASTA |
| | ItmK | N. lolii | 36.8 | | FASTA |
| | ItmP | N. lolii | 25 | | FASTA |
| | ItmQ | N. lolii | 21.9 | | FASTA |
| | AN1598 | A. nidulans | | 4e-81 | BLASTP |
| ItmE | paxC | P. paxilli | 43.1 | 3e-60 | FASTA/BLASTP |
| | atmC | A. flavus | 49.5 | 7e-71 | FASTA/BLASTP |
| | ItmC | N. lolii | 55.8 | | FASTA |
| | ItmD | N. lolii | 37.1 | | FASTA |
| | ItmG | N. lolii | 32.8 | | FASTA |
| | AN8514 | A. nidulans | | 7e-56 | BLASTP |

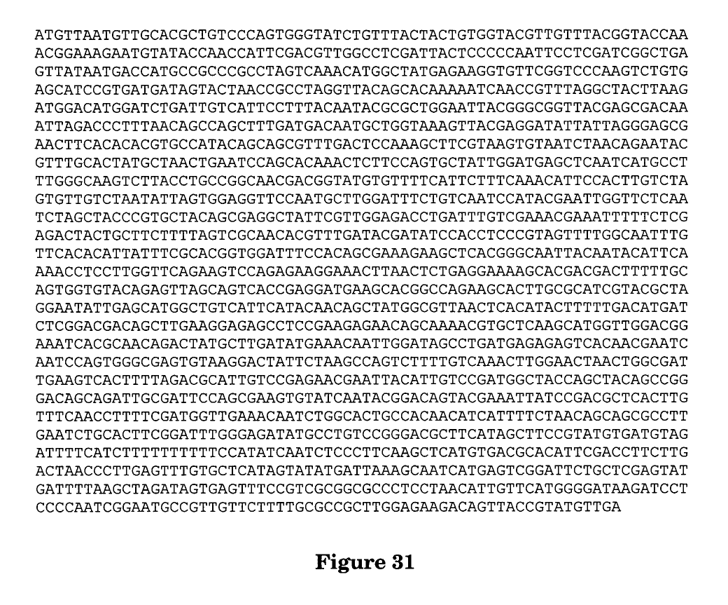

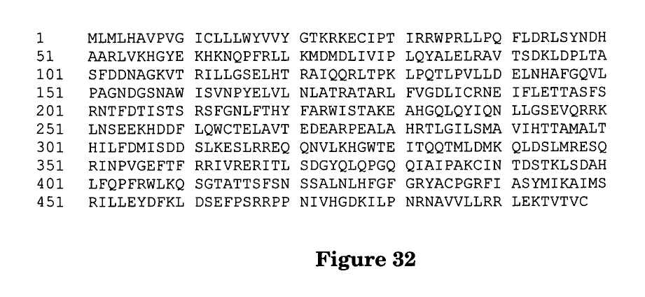

Sequence analysis of the complete ltmP gene, initially identified from EST sequences, showed that it contained five introns (FIGS. 27 and 31) and encodes a polypeptide of 498 amino acids (FIG. 32). LtmP is classified as a cytochrome P450 monooxygenase based on database matches. The placement and phase of four introns, 1, 2, 3 and 4, are conserved with the paxP introns, 1, 3, 4 and 5 (Young et al, 2001); and three introns 1, 3, and 4, conserved with ltmK introns, 1, 4, and 7. LtmP is more similar to PaxP than to PaxQ or ltmK.

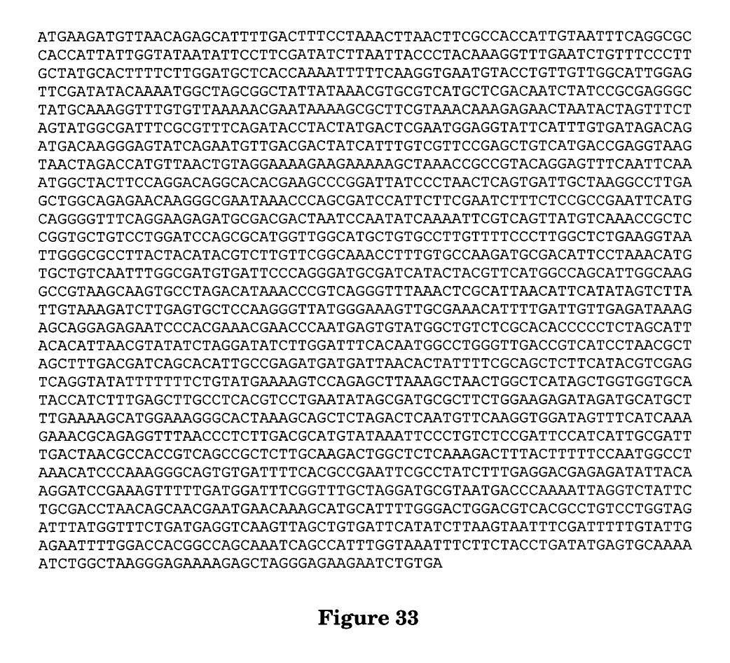

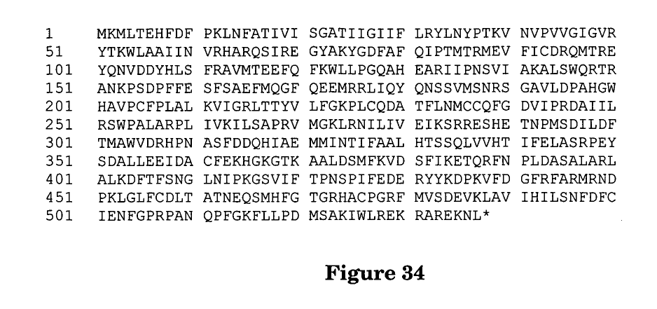

Adjacent to ltmP is ltmQ, a cytochrome P450 monooxygenase gene (FIGS. 28 and 33). The best database match to ltmQ is that of paxQ from P. paxilli and FastA analysis confirmed that LtmQ is more similar to PaxQ than to PaxP (Table 7). The nucleotide sequence of N. lolii ltm Q and deduced amino acid sequence of ltmQ polypeptide shown in FIG. 33 and 34 respectively. The ltmQ gene contains 7 introns (FIGS. 28 and 31) of which six, introns 2 to 7, are conserved in placement and phase with paxQ introns 2, 3, 5, 6, 7 and 8. It appears from ClustalW alignments of the ltm and pax P450 monooxygenases that ltmQ has lost a conserved intron, that is present in the remaining five sequences shown in the alignment, corresponding in position between the current ltmQ introns 3 and 4. The intron boundaries of ltmQ were confirmed by sequence comparison of RT-PCR products amplified using cDNA from endophyte infected plant material and gene specific primers, to the LP19 genomic region.

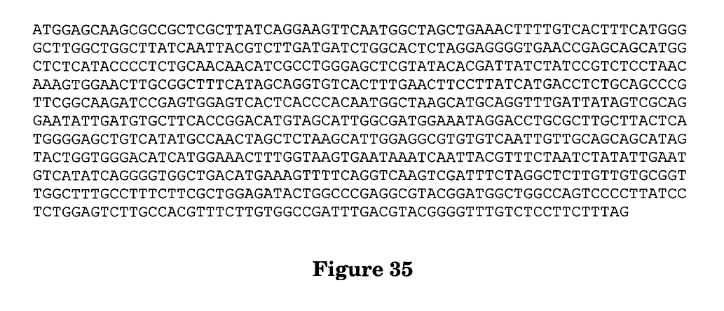

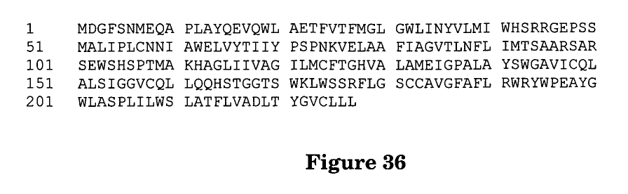

The ltm25 gene has no predicted function and had a best BLASTP match to an uncharacterised gene from F. graminearum FG04594, (accession number EAA72208). Using the tBLASTN algorithm against the public databases the best match was to sec25, a gene recently identified within the P. paxilli pax cluster, but as yet not publicly annotated (Monahan and Scott, unpublished). The ltm25 gene has one intron (FIGS. 27 and 31) that is conserved in placement and phase with the sec25 gene from P. paxilli. The nucleotide sequence of N. lolii ltm Q and deduced amino acid sequence of ltmQ polypeptide shown in FIGS. 35 and 36 respectively.

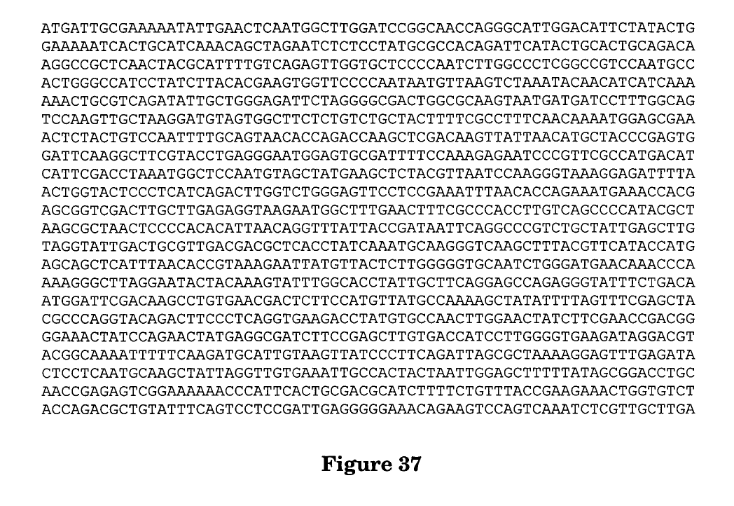

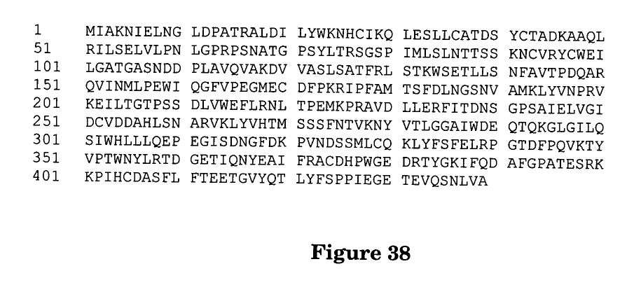

The ltmD gene, had a best BLASTP match to A. nidulans AN8514. The nucleotide sequence of N. lolii ltm D and deduced amino acid sequence of ltmQ polypeptide shown in FIGS. 37 and 38 respectively. FastA analysis was used to compare LtmD to DmaW from Lp1 a Neotyphodium LpTG-2 and PaxD from P. paxilli as the publicly available paxD sequence is not complete (Table 7). This data showed that LtmD is more similar to PaxD than to the DmaW from Neotyphodium LpTG-2. The ltmD gene contains two introns of which the placement and phase of intron 2 is conserved with paxD.

The predicted introns of the five genes contained in ltm cluster 2 were confirmed by sequence comparison of cDNA sequences, generated by RT-PCR using cDNA from endophyte infected plant material, with the genomic sequence. A summary of the intron numbers, intron splice sites and predicted molecular mass in kDa of each gene is shown in Table 8.

Flanking ltm25 is a Rua long terminal repeat (Rua4) and degenerate retrotransposon sequence (FIG. 27). Upstream from ltmP is an AT-rich region that was devoid of obvious open reading frames and no genes were evident from sequence analysis using BLAST searches. Southern analysis with a fragment from this region to EcoRI, Hindlll and Sstl digested DNA showed that this sequence is present in the lolitrem producing strains LP19 and Fl1 but absent from the non-producer E8. Based on Southern analysis there are predicted to be ˜3-5 copies of this sequence contained within the LP19 and Fl1 genomes (data not shown). The presence of AT rich sequences adjacent to ltm25 and ltmP suggested that no additional genes are present at this locus thereby defining the boundaries of ltm cluster 2.

ltm Cluster 3

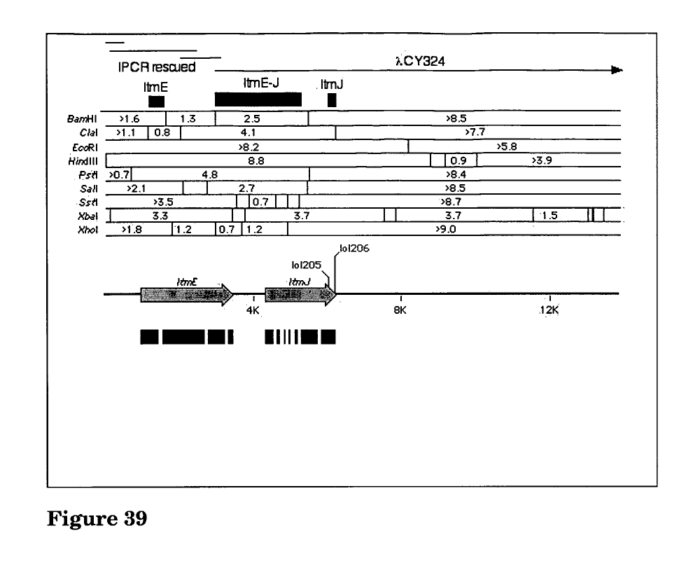

The sequence surrounding ltmJ, a cytochrome P450 monooxygenase initially identified from EST sequence N17, was isolated from the LP19 λGEM-12 genomic library hybridised with the ltmJ fragment, amplified with primers lol205 and lol206. This hybridisation resulted in the isolation of 22 positive clones. Fifteen clones were digested with Hindlll or BamHl and hybridised with the ltmJ fragment to determine clones of interest. Comparison of the restriction enzyme digests and sequencing of these clones, with primers, SP6 and T7, that anneal to the lambda arms, showed that only two identical clones, λCY324 and λCY344, had the correct genomic arrangement based on Southern and PCR analysis. FIG. 39 shows a physical map of the cluster 3 locus. Other lambda clones were rearranged and/or contained unrelated sequences.

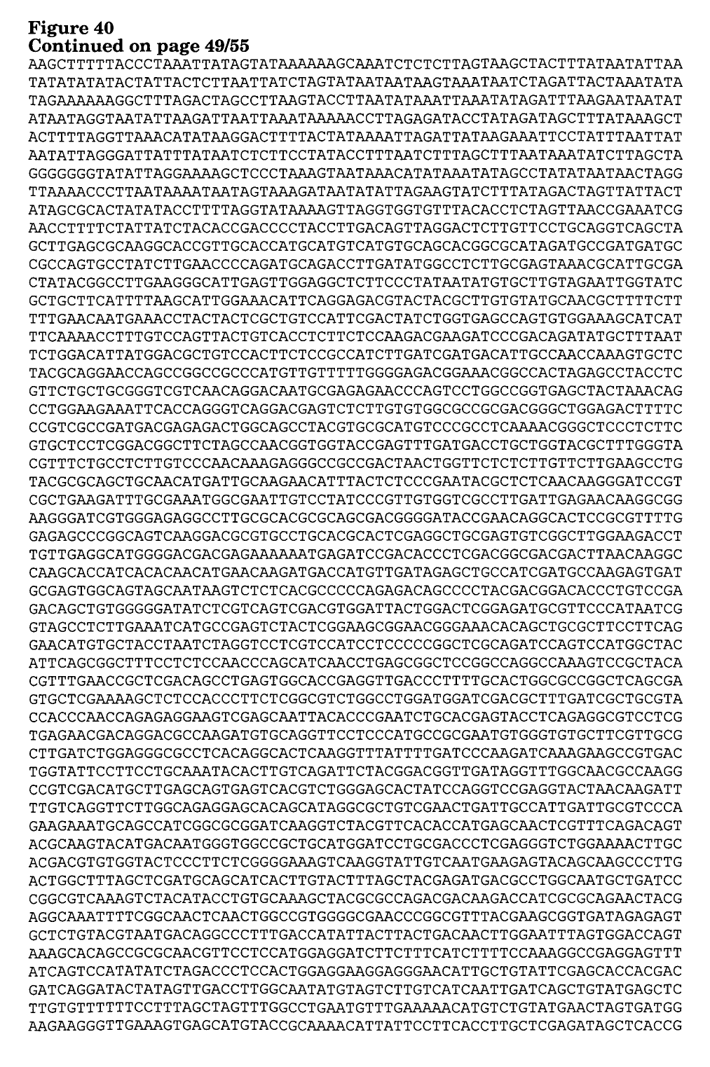

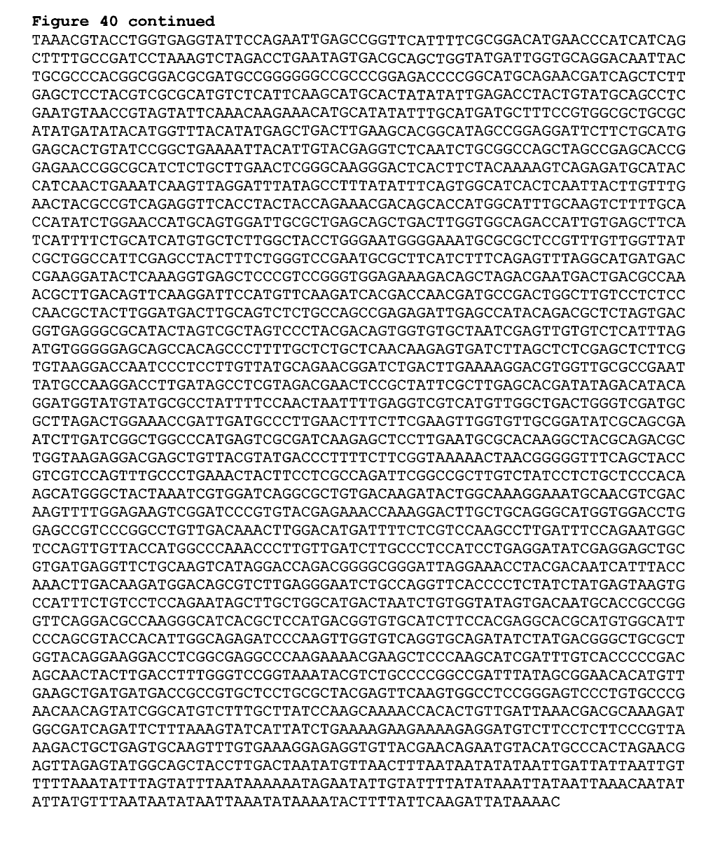

Sequence analysis of λCY346 and λCY324 identified from ltmP and ltmJ hybridisations respectively, were shown to overlap, linking ltm clusters 2 and 3 with a 16-kb AT-rich region separating them. Sequence analysis of this AT-rich region, using the BLASTX analysis of this sequence failed to identify any evidence of potential genes. The strong AT bias of this sequence introduces numerous stop codons strongly suggesting it is non-coding. Additional sequence flanking the left-hand side of λCY324 was extended by IPCR using Clal, Xbal or Hindlll digested then self-ligated LP19 genomic DNA and sequence specific primers. Analysis of ltm cluster 3 sequence (FIG. 40) identified two genes, a cytochrome P450 monooxygenase, ltmJ, and ltmE, a gene that encodes a gene fusion of two prenyl transferases, a ltmC type with a dimethylallyl tryptophan synthase ltmD type (FIG. 28).

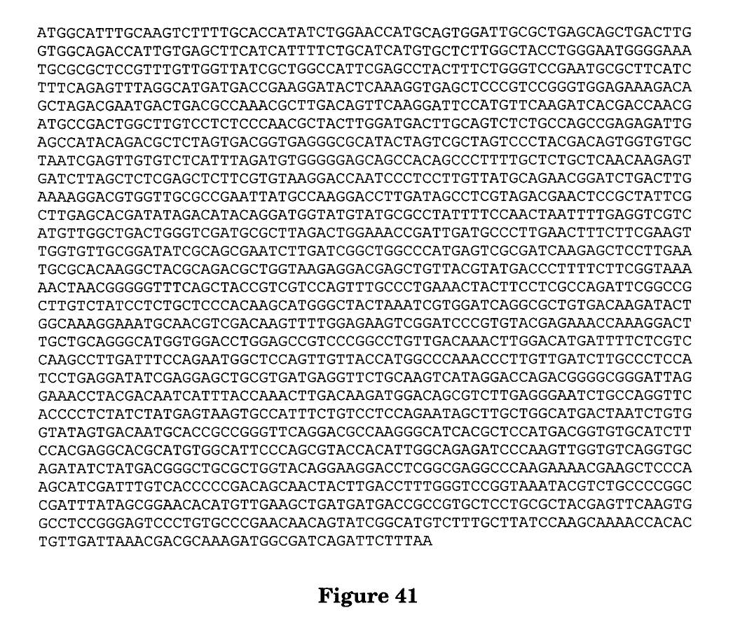

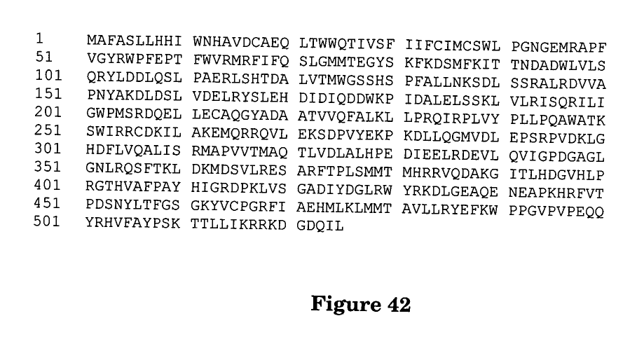

The complete ltmJ gene was contained on λCY234. The nucleotide sequence of N. lolii ltm J and deduced amino acid sequence of ltmQ polypeptide shown in FIG. 41 and 42 respectively. Sequence analysis of ltmJ revealed the presence of six introns of which all are conserved with the introns, 1, 2, 3, 4, 5 and 7, from ltmK located in ltm cluster 1. LtmJ has a best BLASTP match to an A. nidulans AN1598 sequence (Table 8). Of the four N. lolii cytochrome P450 monooxygenase genes identified, LtmJ is most similar to LtmK followed by LtmP then LtmQ (Table 7).

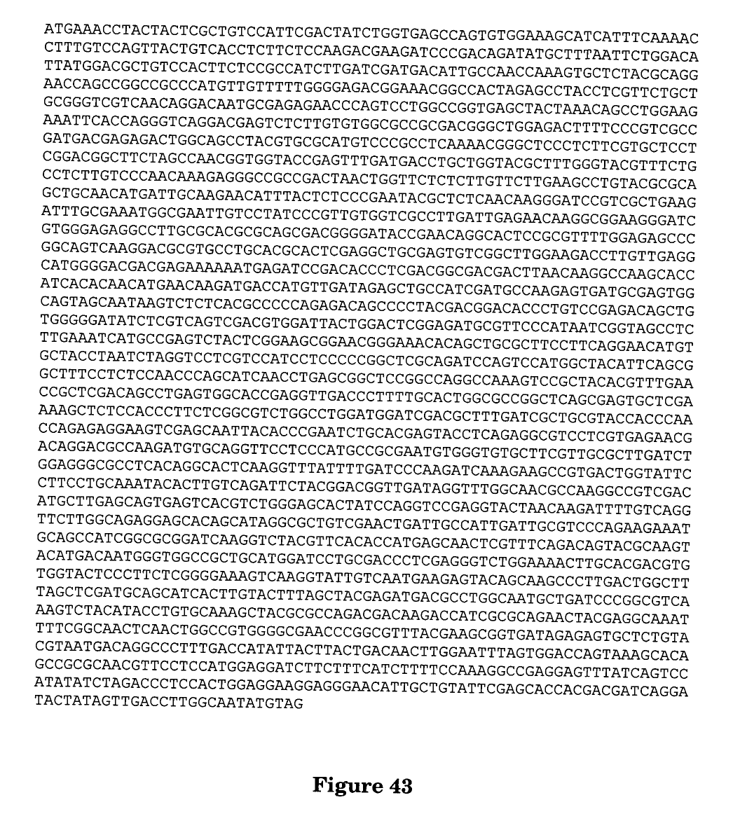

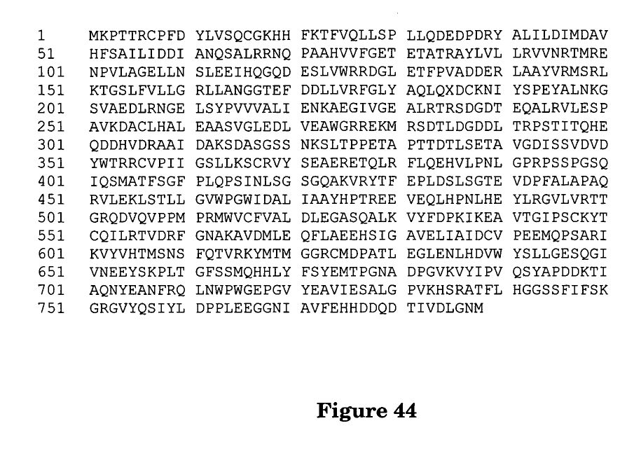

The complete ltmE has significant BLASTP matches to both P. paxilli paxC, and to the A. nidulans gene, AN8514 (Table 7). The nucleotide sequence of N. lolii ltm E and deduced amino acid sequence of ltmQ polypeptide shown in FIGS. 43 and 44 respectively. FastA analysis shows that LtmE is 55.8% identical to LtmC and 37.1% identical to LtmD. The ltmE gene contains 3 introns (FIG. 39) of which intron 1 from the ltmC-like domain is conserved with the placement and phase of ltmC intron, while intron 3, from the ltmD-like domain is possibly conserved with the second ltmD intron.

The library screen with the ltmJ probe isolated two identical clones λCY325 and λCY338 that were rearranged at the T7 end. These clones contained sequence with strong similarity to class V chitin synthase gene, chsV. The complete gene was contained within the clones and sequenced. The gene is approximately 5.7 kb and has two introns (FIG. 39) that are conserved with placement and phase to those found in other fungal chsV genes. The sequence of chsV gene is highly conserved with a significant BLASTP match to Blumeria graminis (accession number AAF04279) (Table 8). However, chsV is not part of ltm cluster 3.

The introns of ltmJ and ltmE from cluster 3 and the chsV from λCY325 and λCY338 were confirmed by RT-PCR using cDNA from endophyte infected plant material. The intron number, intron splice sites and predicted mass in kDa of each gene are summarised in Table 8.

Expression Profiles of the 10 ltm Genes

The expression profiles of the 10-ltm genes, the chsV from λCY325 and the pks adjacent to cluster 1, were characterised in planta and in culture. Previous data showed that the endophyte biomass in planta is approximately 1%. Given that expression of ltmG, ltmM and ltmK were highly up regulated in planta the other genes involved in lolitrem biosynthesis were also expected to follow a similar expression pattern. Random primed cDNA pools were made from mRNA of LP19 infected Nui perennial ryegrass, Fl1 infected meadow fescue, and LP19 and Fl1 grown in liquid culture. The cDNA pools from the two endophyte growth conditions, in planta and in culture were diluted to levels where the endophyte tub2 sequences were amplified to similar levels thereby adjusting the levels of cDNA from endophytes grown in culture to a similar level to that of the endophyte in planta. A dilution series of the cDNA synthesised from mRNA of endophyte grown in culture and cDNA synthesised from mRNA of endophyte infected ryegrass diluted 1/10 were used as templates for the amplification of tub2 with primers T1.1 and T1.2. The expression of the tub2 gene was equivalent between cDNA from endophyte infected ryegrass diluted 1/10 with a 1/2000 or 1/400 cDNA dilution from LP19 or Fl1 culture conditions, respectively.

The expression pattern of each gene was subsequently compared from the cDNA of endophyte infected ryegrass, to that of cDNA from the endophyte alone. The expression of all 10 ltm genes have similar transcript levels indicating that these genes are highly up regulated in planta. No transcript was detected for any of the 10-ltm genes from cDNA of endophytes grown in culture. An additional dilution, 10 fold more concentrated, of cDNA synthesised from the endophyte culture was included in the experiment to unequivocally show that the expression patterns from the culture condition did not contain ltm transcripts. The expression of the chsV is similar to that of tub2 where the gene appears to be constitutively expressed in planta and in culture. No evidence of pks expression is seen in either endophyte infected plant material or in culture.

### EXAMPLE 9

### Functional Analysis of ltmC

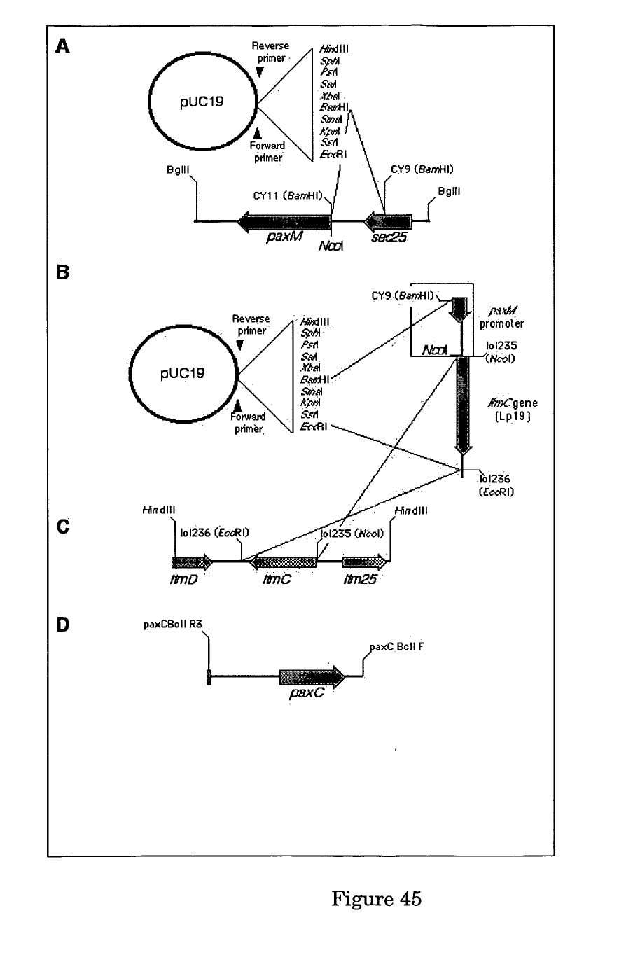

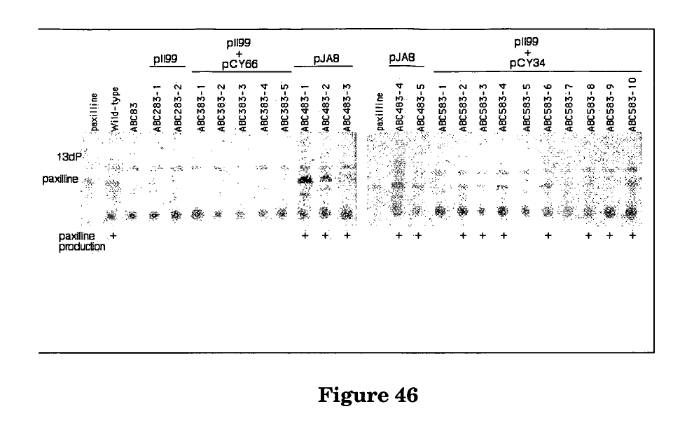

Functional characterisation of ltmC was determined by complementation of the P. paxilli paxC deletion mutant, ABC83. The ABC83 mutant is blocked early in the paxilline biosynthesis pathway and therefore unable to synthesis indole-diterpenoids (data not shown). To express ltmC in the P. paxilli background, the gene was put under the control of the paxM promoter in pPN1851 (FIGS. 45 and 29). The sequences of the LP19 and Fl1 ltmC genes are identical, therefore the ltmC gene was amplified from LP19 genomic DNA using the high fidelity proofreading enzyme, Platinum Pfx (Invitrogen), with primers lol235 and lol236. These primers, incorporate Ncol and EcoRI restriction enzyme recognition sites, respectively. The 1242 bp PCR fragment, containing the ltmC gene and 109-bp of 3′ untranslated region, was digested with Ncol and EcoRI and directionally cloned into pPN1851, resulting in plasmid pCY34 (FIG. 45). The ltmC gene was fused to the paxM promoter at the ATG translational start site using the restriction enzyme Ncol. The translational fusion that results in creating an Ncol site in the ltmC gene caused a single base change where the second codon of ltmC has a conservative replacement of threonine in the wild-type gene, to alanine in the fused gene. A 3.5 kb Hindlll fragment from λCY315 was cloned into a pUC118 vector resulting in plasmid pCY66 (FIG. 45). This 3.5 kb Hindlll fragment contained the complete LP19 ltmC gene under the control of its native promoter. Protoplasts of ABC83 were transformed with pll99 and pJA8, containing an endogenous paxC fragment, or co-transformed with pCY34 and pll99, or pCY66 and pll99, and transformants selected on geneticin. Approximately 5-10 stable P. paxilli ABC83 transformants were colony purified and subsequently screened by TLC analysis for their ability to produce paxilline (FIG. 46).

TLC analysis of the wild-type P. paxilli indole-diterpenoid extraction showed intense green bands that have the same Rf as paxilline, paspaline and 13-desoxypaxilline (FIG. 46). The ABC83 paxC mutant, used for the transformations, was unable to produce any indole-diterpene (FIG. 46). The ABC83 transformants containing pll99 are unable to complement the paxC mutation and are therefore paxilline negative (FIG. 46; samples ABC283-#). The ABC83 transformants co-transformed with plasmids pll99 and pCY66 with the LP19 ltmC gene under the control of the native LP19 promoter are unable to complement the paxC mutation and are paxilline negative (FIG. 46; samples ABC383-#). All five ABC83 transformants containing the endogenous paxC gene on plasmid pJA8 were able to complement the paxC deletion phenotype (FIG. 46; samples ABC483-#). Seven of the 10 transformants containing ltmC under the control of the paxM promoter are able to produce paxilline (FIG. 46; samples ABC583-#). The TLC analysis was confirmed by HPLC analysis. This data confirmed that ltmC is a functional orthologue of paxC.

### EXAMPLE 10

### Methods for Expression of Lolitrem Genes in Transgenic Plants

Once the lolitrem biosynthetic gene cluster is fully characterized it is possible to modify the fungal genes to enable expression in transgenic plants. Fungal genes containing introns will not be correctly spliced in plants so cDNAs for each gene need to be obtained. Those familiar with the art will know it is possible to isolate cDNAs using cDNA synthesis kits such as those described in Example 6. The cDNAs need to be cloned into a vector that contains a plant promoter and terminator sequence. Those familiar with the art know that there are many possible promoter and terminator combinations. A common example is the 35S promoter from Cauliflower Mosaic Virus (Odell et al., 1985). These modified fungal genes can then be transformed into plant species using either the gene gun or agrobacterium. Two methods are described below.

Transformation of Lolium perenne by Microprojectile Bombardment of Embryogenic Callus

It is possible to use perennial ryegrass L. perenne as a model system for monocot plant species. Demonstration of biosynthesis of indole diterpenes in this species can be extrapolated to other monocot species such as wheat, rice and corn.

Materials

florally induced tillers of Lolium perenne

Na-hypochlorite (5% available chlorine)

sterile ddH₂O 100 mm Petri plates containing LP5 medium*

100mm Petri plates containing LP3-OS medium

100mm Petri plates containing LP3 medium

100mm Petri plates containing LP3 medium+200 mg/L Hygromycin (Hm)

100mm Petri plates containing MSK medium+200 mg/L Hm

250 ml culture vessels containing MSO medium+200mg/L

Hygromycin stock solution (50 mg/ml in PDS, sterile)

Procedure

1) Harvest and surface sterilise floral tillers of Lolium perenne in 5% available chlorine Na-hypochlorite for 15 minutes using a Mason jar (or equivalent) under constant agitation.

2) Rinse tillers with autoclaved ddH₂O.

3) Aseptically dissect floral meristems.

4) Culture meristems on callus induction medium LP5 (16-20 explants per plate) and incubate in the dark for four to six weeks.

5) On the day of transformation transfer embryogenic callus material to high osmotic medium LP3-OS. Arrange approximately 4 cm² of calli in the centre of the Petri dish.

6) Incubate calli for 4-6 hours at room temperature.

7) Prepare particles and perform biolistic transformation following the protocol: “Biolistic Transformation of Lolium perenne with the Bio-Rad Particle Delivery System (PDS)”. Plasmids are co-transformed. One plasmid (pAcH1) contains the hygromycin phosphotransferase gene conferring resistance to the antibiotic hygromycin expressed from the rice actin promoter and the second plasmid contains the genetic construct of interest for transformation. Plasmids are mixed in a one to one ratio at 1 μg/μL and simultaneously coated onto the microcarriers.

8) Incubate bombarded calli on high osmotic medium LP3-OS for an additional 12-16 hours (overnight) at 25° C. in the dark.

9) Transfer bombarded calli to LP3 medium and incubate for 48 hours at 25° C. in the dark

10) Plate calli on selection medium (LP3+200 mg/l Hygromycin (Hm)). Incubate at 25° C. in the dark on selection medium for two weeks.

11) Transfer all Hm-resistant callus material to regeneration medium MSK+200 mg/l Hm and incubate for four weeks at 25° C. under a 16 hour photoperiod.

12) Transfer developed shoots to MSO+200 mg/l Hm and incubate for another two to four weeks at 25° C. under 16hour photoperiod.

13) Screen by PCR Hm-resistant plants growing on MSO+200 mg/L Hm.

Microprojectile Bombardment of Lolium perenne with the Bio-Rad Particle Delivery System (PDS-1000/He)

Taken from the PDS-100/He manual. These procedures were developed by Sanford et al. (1992).

Materials and Solutions

Bio-Rad Biolistic® PDS-1000/He Particle Delivery System

Rupture disks (900 PSI)

Macrocarriers

Macrocarrier holders

Microcarriers (1.0 μm)

Stopping screens

Autoclaved 1.5 ml eppendorf tubes

Micropipette tips

Vortex and microfuge

Torque wrench tool

Pen vac

70% Ethanol

Absolute Ethanol

2.5 M CaCl₂

100 mM Spermidine

(A) Microcarrier Preparation

For 120 bombardments using 500 μg per bombardment.

1. In a 1.5 ml microfuge tube, weigh out 60 mg of microparticles.

2. Add 1 ml of 70% ethanol, freshly prepared.

3. Vortex on a platform vortexer for 3-5 minutes.

4. Incubate for 15 minutes.

5. Pellet the microparticles by spinning for 5 seconds in a microfuge.

6. Remove the liquid and discard.

7. Repeat the following steps three times:
a. Add 1 ml of sterile water
b. Vortex for 1 minute
c. Allow the particles to settle for 1 minute
d. Pellet the microparticles by spinning for 2 seconds in a microfuge.
e. Remove the liquid and discard.

8. Add sterile 50% glycerol to bring the microparticle concentration to 60 mg/ml (assume no loss during preparation).

9. Store the microparticles at room temperature for up to 2 weeks.

(B) Coating DNA onto Microcarriers

The following procedure is sufficient for six bombardments; if fewer bombardments are needed, prepare enough microcarriers for three bombardments by reducing all volumes by one half. When removing aliquots of microcarriers, it is important to vortex the tube containing the microcarriers continuously in order to maximise uniform sampling.

1. Vortex the microcarriers prepared in 50% glycerol (60 mg/ml) for 5 minutes on a platform vortexer to resuspend and disrupt agglomerated particles.

2. Remove 50 μl (3 mg) of microcarriers to a 1.5 ml microfuge tube.

3. While vortexing vigorously, add in order:
5 μl DNA (1 μg/μl)
50 μl CaCl₂ (2.5 M)
20 μl spermidine (0.1 M)

4. Continue vortexing for 2-3 minutes

5. Allow the microcarriers to settle for 1 minute

6. Pellet the microcarriers by spinning for 2 seconds in a microfuge

7. Remove the liquid and discard

8. Add 140 μl of 70% ethanol without disturbing the pellet

9. Remove the liquid and discard

10. Add 140 μl of 100% ethanol without disturbing the pellet

11. Remove the liquid and discard

12. Add 48 μl of 100% ethanol

13. Gently resuspend the pellet by tapping the side of the tube several times, and then by vortexing at low speed for 2-3 seconds

14. Remove six 6 μl aliquots of microcarriers and transfer them to the centre of a macrocarrier. An effort is made to remove equal amounts (500 μg) of microcarriers each time and to spread them evenly over the central 1 cm of the macrocarrier using the pipette tip. Desiccate immediately.

C) Bombardment Procedure

1) Open valve of helium cylinder

2) Adjust helium regulator by turning the helium pressure regulator to 200 PSI above chosen rupture disk (e.g. if a 900 PSI rupture disk will be used, the working pressure has to be adjusted to 1100 PSI)

3) Turn on vacuum pump

4) Place 900 psi rupture disk in the rupture disk-retaining cap. Screw on and tighten retaining cap.

5) Place macrocarriers in sterile macrocarrier holder

6) Place stop screen and macrocarrier holder in the launch assembly, tighten screw lid and place below rupture disk-retaining cap. Launch assembly should be set to a Gap distance of ¼ inch and macrocarrier travel distance of 11 mm.

7) Place tissue sample at a target distance of 90 mm.

8) Turn on main switch of PDS

9) Apply vacuum to 27 inches of Hg

10) Hold vacuum and press “fire” button until shot is performed (automatic)

11) Release “fire” button and vent chamber

12) After shooting close valve of helium cylinder and loosen pressure valve

TABLE 7
Compositions of the media used

| Media component | LP3 | LP5 | LP3-OS | MSK | MS0 |
|---|---|---|---|---|---|
| Macro elements (mg/l final concentration) | | | | | |
| KNO₃ | 1900 | 1900 | 1900 | 1900 | 1900 |
| NH₄NO₃ | 1650 | 1650 | 1650 | 1650 | 1650 |
| CaCl₂ × 2H₂O | 440 | 440 | 440 | 440 | 440 |
| MgSO₄ × 2H₂OKH₂PO₄ | 370 | 370 | 370 | 370 | 370 |
| KCl | 170 | 170 | 170 | 170 | 170 |
| Micro elements (mg/l final concentration) | | | | | |
| Na₂EDTA | 37.3 | 37.3 | 37.3 | 37.3 | 37.3 |
| FeSO₄ × 7H₂O | 27.8 | 27.8 | 27.8 | 27.8 | 27.8 |
| H₃BO₃ | 6.2 | 6.2 | 6.2 | 6.2 | 6.2 |
| KI | 0.83 | 0.83 | 0.83 | 0.83 | 0.83 |
| MnSO_{4 × H}₂O | 16.9 | 16.9 | 16.9 | 16.9 | 16.9 |
| ZnSO₄ × 7H₂O | 8.6 | 8.6 | 8.6 | 8.6 | 8.6 |
| CuSO₄ × 5H₂O | 0.025 | 0.025 | 0.025 | 0.025 | 0.025 |
| Na₂MoO₄ × 2H₂O | 0.25 | 0.25 | 0.25 | 0.25 | 0.25 |
| CoCl₂ × 6H₂O | 0.025 | 0.025 | 0.025 | 0.025 | 0.025 |
| Carbohydrates (g/l final concentration) | | | | | |
| Maltose | 30 | 30 | 30 | 30 | 30 |
| D-Mannitol | | | 64 | | |
| Hormones (mg/l final concentration) | | | | | |
| 2,4-D | 3.0 | 5.0 | 3.0 | | |
| Kinetin | | | | 0.2 | |
| Vitamins (mg/l final concentration) | | | | | |
| Pyridoxine HCl | 0.5 | 0.5 | 0.5 | 0.5 | |
| Thiamine HCl | 0.1 | 0.1 | 0.1 | 0.1 | |
| Nicotinic acid | 0.5 | 0.5 | 0.5 | 0.5 | |
| Myo-Inositol | 100 | 100 | 100 | 100 | |
| Other organics (mg/l final concentration) | | | | | |
| Glycine | 2 | 2 | 2 | 2 | 2 |

Culture Media

Weights and volumes required of each individual ingredient are specified in Table 7. Adjust media pH to 5.8 with KOH. The addition of a solidifying agent is required. Use agarose (for LP3, LP5 and LP3-OS) and 0.8% (w/v) Agar for MSO and MSK prior to sterilising. Media LP3, LP5 and MSK are modified from Murashige and Skoog (1962).

Expression of Chimeric Genes in Corn Cells

A chimeric gene comprising a lolitrem cDNA encoding in sense orientation with respect to the promoter that is located 5′ to the cDNA fragment, and a terminator 3′ to the cDNA fragment, can be constructed. The cDNA fragment of this gene may be generated by polymerase chain reaction (PCR) of the cDNA clone using appropriate oligonucleotide primers. Cloning sites can be incorporated into the oligonucleotides to provide proper orientation of the DNA fragment when inserted into the digested vector as described below. Amplification is then performed in a standard PCR reaction. The amplified DNA is then digested with restriction enzymes and fractionated on an agarose gel. The appropriate band can be isolated from the gel and combined with Vector and insert DNA can be ligated at 15° C. overnight, essentially as described (Sambrook). The ligated DNA may then be used to transform E.Coli XL1-Blue (Epicurian Coli XL-1 Blue™, Stratagent). Bacterial transformants can be screened by restriction enzyme digestion of plasmid DNA and limited nucleotide sequence analysis using the dideoxy chain termination method (Sequenase™ DNA sequencing Kit; US Biochemical). The resulting plasmid construct would comprise a chimeric gene encoding in the 5′ to 3′ direction promoter, a cDNA encoding and the 3′ region containg a terminator.

The chimeric gene described above can then be introduced into cells by the following procedure. Immature corn embryos can be dissected from developing caryopses derived from crosses of the inbred corn lines. The embryos are isolated 10 to 11 days after pollination when they are 1.0 to 1.5 mm long. The embryos are then placed with the axis-side facing down and in contact with agarose-solidified N6 medium (Chu et al. (1975) Sci. Sin Peking 18:659-668). The embryos are kept in the dark at 27° C. Friable embryogenic callus consisting of undifferentiated masses of cells with somatic proembryoids and embryoids borne on suspensor structures proliferates from the scutellum of these immature embryos. The embryogenic callus isolated from the primary explant can be cultured on N6 medium and sub-cultured on this medium every 2 to 3 weeks.

The particle bombardment method (Klein et al. (1987) Nature 327:70-73) may be used to transfer genes to the callus culture cells. According to this method, gold particles (1 um in diatmeter) are coated with DNA using the following technique. Ten ug of plasmid DNAs are added to 50 uL of a suspension of gold particles (60mg per mL). Calcium chloride (50 uL of a 2.5 M solution) and spermidine free base (20 uL of a 1.OM solution) are added to the particles. The suspension is vortexed during the addition of these solutions. After 10 minutes, the tubes are briefly centrifuged (5 sec at 15,000 rpm) and the supernatant removed. The particles are resuspended in 200 uL of absolute ethanol, centrifuged again and the supernatant removed. The ethanol rinse is performed again and the particles resuspended in a final volume of 30 uL of ethanol. An aliquot (5 uL) of the DNA-coated gold particles can be placed in the centre of a Kapton™ flying disc (Bio-Rad Labs). The particles are then accelerated into the corn tissue with a Biolistic™ PDS-1000/He (Bio-Rad Instruments Hercules Calif.), using a helium pressure of 1000 psi, a gap distance of 0.5 cm and a flying distance of 1.0 cm.

For bombardment, the embryogenic tissue is placed on filter paper over agarose-solidified N6 medium. The tissue is arranged as a thin lawn and covered a circular area of about 5 cm in diameter. The Petri dish containing the tissue can be placed in the chamber of the PDS-1000/He approximately 8 cm from the stopping screen. The air in the chamber is then evacuated to a vacuum of 28 inches of Hg. The macrocarrier is accelerated with a helium shock wave using a rupture membrane that bursts when the He pressure in the shock tube reaches 1000 psi.

Seven days after bombardement the tissue can be transferred to N6 medium that contains a selection. The tissue continues to grow slowly on this medium. After an additional 2 weeks the tissue can be transferred to fresh N6 medium containing the selection. After 6 weeks, areas of about 1 cm in diameter of actively growing callus can be identified on some of the plates containing the selective medium. These calluses may continue to grow when sub-cultured on the selective medium.

Plants can be regenerated from the transgenic callus by first transferring clusters of tissue to N6 medium supplemented with 0.2 mg per liter of 2,4-D. After two weeks the tissue can be transferred to regeneration medium (Fromm et al., (1990) BioTechnology 8:833-839.

Aspects of the present invention have been described by way of example only and it should be appreciated that modifications and additions may be made thereto without departing from the scope thereof.

### EXAMPLE 11

### Use of Ltm Gene Sequence Information to Characterise Endophyte Strains

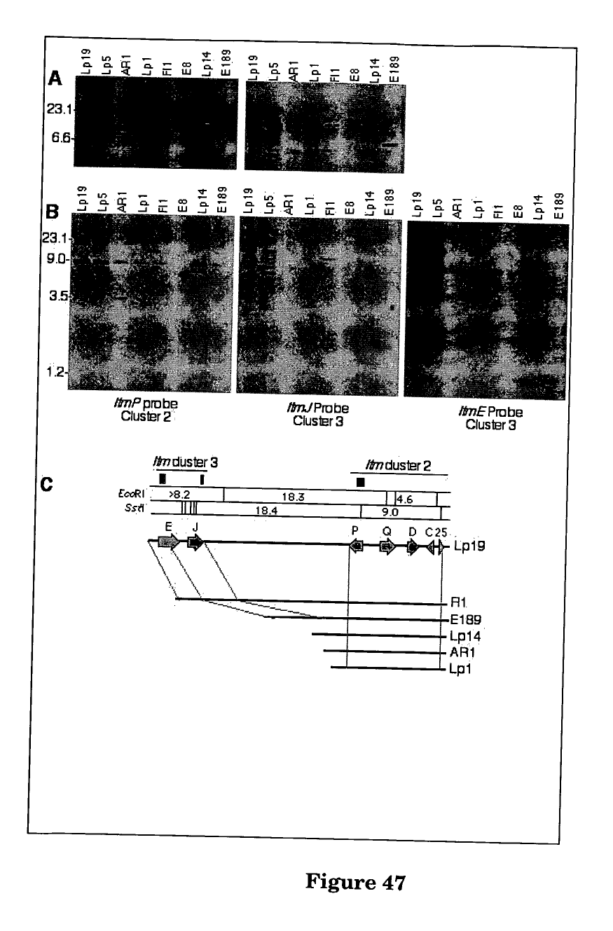

Identification of the ltm gene cluster provides the opportunity to chatacterise endophyte strains genetically and correlate gene information with the chemical phenotype. A number of Neotyphodium and Epichloë strains do not prodice lolitrem B. In this example we demonstrate that strains Lp1, Lp14 and AR1 that are all lolitrem B minus (reference) lack the ltmE and ltmJ genes of cluster 3. Southern analysis (FIGS. 47 A and B) showed that these strains did not hybridise to probes for the ltmJ or ltmE genes. Genes for Cluster 2 were present (FIGS. 3, 27, 39 and 47). This suggests that LtmJ and or LtmE are important for the biosynthesis of lolitrem B. However not all the lolitrem biosynthesis pathway is absent in these strains (Clusters 1 and 2) suggesting that intermediate compounds may be produced.

The lolitrem producers were N. lolii LP19 and Lp5, and E. festucae Fl1. N. lolii Lp14 produces janthitrems, compounds structurally related to the lolitrems. The lolitrem non-producers were N. lolii AR1, Neotyphodium spp. Lp1, and E. typhina E8. The chemotype of E. festucae E189 is unknown. These isolates were screened for the presence of genes ltmP, ltmJ and ltmE as strains had previously been shown by Southern analysis to have differences in this region. Very little nucleotide sequence diversity is found between the asexual N. lolii, Lp19, and sexual E. festucae, Fl1, across the ltm genes in cluster 1, therefore standard hybridisation conditions were used.

The ltmP probe hybridised to seven of the eight strains screened (FIG. 47). E8 is the only strain negative for ltmP hybridisation. The ltmP probe contains a Sstl site and therefore hybridises to two fragments with a 9-kb hybridising band common to the seven strains that contain ltmP. This band in LP19 contains the genes ltmC, ltmD, ltmQ, and partial sequences of ltm25 and ltmP (FIG. 47). Sequence diversity is seen amongst the seven strains that contained ltmP as the bands of the EcoRI digested DNA and the second Sstl hybridising fragment were of varying sizes. Lp5 has two copies of ltmP seen clearly as two hybridising bands in the EcoRI digested DNA.

The exact approach is described as follows: the ltmJ probe hybridised to four, Lp19, Lp5, Fl1 and E189, of the eight strains screened (FIG. 47). E8, Lp1, AR1 and Lp14 are all negative for ltmJ hybridisation. Lp5 contains two copies of ltmJ, one that hybridises to a Sstl fragment the same size as LP19 (˜18 kb) and a second 8.5 kb fragment. The ltmE probe hybridises to the same four strains, Lp19, Lp5, Fl1 and E189, as that of the ltmJ hybridisation (FIG. 47). E8, Lp1, AR1 and Lp14 are all negative for ltmE hybridisation. LP19 and Lp5 have the same sized ltmE hybridising band of ˜20 kb. Fl1 and E189 have ltmE hybridising bands of 9 kb and 1.2 kb, respectively. The AT-rich region between clusters 2 and 3 is smaller in Fl1 and E189 than LP19 based on the sizes of the hybridising bands with the ltmP and ltmJ probes (FIG. 47). Absence of ltmJ and ltmE in Lp1, AR1, Lp14 and E8 correlated with a lolitrem B negative phenotype, suggesting that these two genes are specific for lolitrem biosynthesis. A schematic diagram of the cluster 2 and 3 regions from strains used in the Southern analysis is shown in FIG. 47. Attempts were made by IPCR to isolate the regions from Lp1, Lp14 and AR1 that flank the deletions but this was unsuccessful.

Similar approaches can be adopted to characterise further strains. Chemical analysis can be linked to the presence or absence of specific genes described in this specification. In addition to Southern analysis, a number of approaches could be used to detect specific genes including PCR and/or sequence analysis.

### REFERENCES

Acklin W, Weibel F, Arigoni D (1977) Zur biosynthese von paspalin und verwandten metaboliten aus Claviceps paspali. Chimia 31:63
Altschul S F, Gish W, Miller W, Myers E W, Lipman D J (1990) Basic local alignment search tool. J Mol Biol 215:403-410
Altschul S F, Madden T L, Schaffer M, Zhang J, Zhang Z, Miller W, Lipman D J (1997) Gapped BLAST and PSI-BLAST: a new generation of protein database search programs. Nucl Acids Res 25:3389-3402
Barbato C, Calissano M, Pickford A, Romano N, Sandmann G, Macino G (1996) Mild RIP—an alternative method for in vivo mutagenesis of the albino-3 gene in Neurospora crassa. Mol Gen Genet 252:353-361
Bush L P, Wilkinson H H, Schardl C L (1997) Bioprotective alkaloids of grass-fungal endophyte symbioses. Plant Physiol 114:1-7
Carroll A M, Sweigard J A, Valent B (1994) Improved vectors for selecting resistance to hygromycin. Fungal Genet News 22
Chen A P, Kroon P A, Poulter C D (1994) Isoprenyl diphosphate synthases: protein sequence comparisons, a phylogenetic tree, and predictions of secondary structure. Protein Sci 3:600-607
Cole R J, Cox R H (1981) Handbook of toxic fungal metabolites. Academic Press, London
de Jesus A E, Gorst-Allman C P, Steyn P S, van Heerden F R, Vleggar R, Wessels P L, Hull W E (1983) Tremorogenic mycotoxins from Penicillium crustosum. Biosynthesis of Penitrem A. J. Chem. Soc. Perkin Trans. 1863-1868
Eggink G, Engel H, Vriend G, Terpstra P, Witholt B (1990) Rubredoxin reductase of Pseudomonas oleovorans: structural relationship to other flavoprotein oxidoreductases based on one NAD and two FAD fingerprints. J Mol Biol 212:135-142
Fletcher L R (1999) “Non-toxic” endophytes in ryegrass and their effect on livestock health and production. In: Woodfield D R, Matthew C (eds) Ryegrass endophyte: an essential New Zealand symbiosis. Napier, New Zealand, pp. 133-139
Gatenby W A, Munday-Finch S C, Wilkins A L, Miles C O (1999) Terpendole M, a novel indole-diterpenoid isolated from Lolium perenne infected with the endophytic fungus Neotyphodium lolii. J Agric Food Chem 47:1092-1097
Gloer J B (1995) Antiinsectan natural products from fungal sclerotia. Acc Chem Res 28:343-350
Graham-Lorence S E, Peterson J A (1996) Structural alignments of P450s and extrapolations to the unknown. Methods Enzymol 272:315-326
Higgins D, Thompson J N, Gibson T J, Thompson J D, Higgins D G, Gibson T J (1994) CLUSTAL W: improving the sensitivity of progressive multiple sequence alignment through sequence weighting, position-specific gap penalties and weight matrix choice. Nucl Acids Res 22:4673-4680
Huang X-H, Tomoda H, Nishida H, Masuma R, Omura S (1995) Terpendoles, novel ACAT inhibitors produced by Albophoma yamanashiensis. I. Production, isolation and biological properties. J Antibiotics 48:1-4
Keller N P, Hohn T M (1997) Metabolic pathway gene clusters in filamentous fungi. Fungal Genet Biol 21:17-29
Knaus H-G, McManus O B, Lee S H, Schmalhofer W A, Garcia-Calvo M, Helms L M H, Sanchez M, Giangiacomo K, Reuben J P, Smith A B, Kaczorowski G J, Garcia M L (1994) Tremorgenic indole alkaloids potently inhibit smooth muscle high-conductance calcium-activated channels. Biochemistry 33:5819-5828
Laakso J A, Gloer J B, Wicklow D T, Dowd P F (1992) Sulpinines A-C and secopenitrem B: new antiinsectan metabolites from the sclerotia of Aspergillus sulphurous. J Org Chem 57:2066-2071
Latch G C M, Christensen M J (1985) Artificial infection of grasses with endophytes. Annals of Applied Biology 107:17-24
Laws I, Mantle P G (1989) Experimental constraints in the study of the biosynthesis of indole alkaloids in fungi. J Gen Microbiol 135:2679-2692
Li C, Gloer J B, Wicklow D T, Dowd P F (2002) Thiersinines A and B: novel antiinsectan indole diterpenoids from a new fungicolous Penicillium species (NRRL 28147). Org Lett 4:3095-3098Mantle P G (1987) Secondary metabolites of Penicillium and Acremonium. In: Peberdy J F (ed), Penicillium and Acremonium. Plenum Press, New York, pp. 161-243
Mantle P G, Weedon C M (1994) Biosynthesis and transformation of tremorgenic indole-diterpenoids by Penicillium paxilli and Acremonium lolii. Phytochemistry 36:1209-1217
McMillan L K, Carr R L, Young C A, Astin J W, Lowe R G T, Parker E J, Jameson G B, Finch S C, Miles C O, McManus O B, Schmalhofer W A, Garcia M L, Kaczorowski G J, Goetz M A, Tkacz J S, Scott B (2003) Molecular analysis of two cytochrome P450 monooxygenase genes required for paxilline biosynthesis in Penicillium paxilli and effects of paxilline intermediates on mammalian maxi-K ion channels. Mol Gen Genom 270:9-23
Mende K, Homann V, Tudzynski B (1997) The geranylgeranyl diphosphate synthase gene of Gibberella fujikuroi: isolation and expression. Mol Gen Genet 255:96-105
Munday-Finch S C, Wilkins A L, Miles C O (1996) Isolation of paspaline B, an indole-diterpenoid from Penicillium paxilli. Phytochemisty 41:327-332
Ondeyka J G, Helms G L, Hensens O D, Goetz M A, Zink D L, Tsipouras A, Shoop W L, Slayton L, Dombrowski A W, Polishook J D, Ostlind D A, Tsou N N, Ball R G, Singh S B (1997) Nodulisporic acid A, a novel and potent insecticide from a Nodulisporum sp. Isolation, structure determination and chemical transformations. J Chem Soc Chem Commun 119:8809-8816
Parker E J, Scott DB (2004) Indole-diterpene biosynthesis in ascomycetous fungi. In: An Z (ed), Handbook of Industrial Mycology. Marcel Dekker, New York,
Pearson W R, Lipman D J (1988) Improved tools for biological sequence comparison. Proc Natl Acad Sci USA 85:2444-2448
Popay A J, Hume D E, Baltus J G, Latch G C M, Tapper B A, Lyons T B, Cooper B M, Pennell C G, Eerens J P J, Marshall S L (1999) Field performance of perennial ryegrass (Lolium perenne) infected with toxin-free fungal endophytes (Neotyphodium spp.). In: Sambrook J, Fritsch E F, Maniatis T (1989) Molecular cloning: a laboratory manual. Cold Spring Harbor Laboratory Press, Cold Spring Harbor, N.Y.
Sanger F, Nicklen S, Coulson A R (1977) DNA sequencing with chain-terminating inhibitors. Proc Natl Acad Sci USA 74:5463-5467
Schardl C L (2001) Epichloë festucae and related mutualistic symbionts of grasses. Fungal Genet Biol 33:69-82
Schardl C L (1996) Epichloë species: fungal symbionts of grasses. Ann Rev Phytopathol 34:109-130
Scott B (2001) Epichloë endophytes: symbionts of grasses. Curr Opin Microbiol 4:393-398
Scott D B, Jameson G B, Parker E J (2003) Isoprenoids: gene clusters and biosynthetic puzzles. In: Tkacz J S, Lange L (eds), Advances in fungal biotechnology for industry, agriculture and medicine. Kluwer Academic/Plenum Publishers, New York,
Selala M I, Laekeman G M, Loenders B, Masuka A, Herman A G, Schepens P (1991) In vitro effects of tremorgenic mycotoxins. J Nat Products 54:207-212
Spiering M J, Davies E, Tapper B A, Schmid J, Lane G A (2002) Simplified extraction of ergovaline and peramine for analysis of tissue distribution in endophyte-infected grass tillers. J Agricultural and Food Chem 50:5856-5862.
Steyn P S, Vleggaar R (1985) Tremorgenic mycotoxins. Prog Chem Organic Natural Products 48:1-80
Tudzynski B, Hölter K (1998) Gibberellin biosynthetic pathway in Gibberella fujikuroi: evidence for a gene cluster. Fungal Genet Biol 25:157-170
Vallon O (2000) New sequence motifs in flavoproteins: evidence for common ancestry and tools to predict structure. Proteins: Structure, Function and Genetics 38:95-114
Wang J, Machado C, Panaccione D G, Tsai H-F, Schardl C L (2004) The determinant step in ergot alkaloid biosynthesis by an endophyte of perennial ryegrass. Fungal Genet Biol
Wierenga R K, Terpstra P, Hol W G J (1986) Predictions of the occurrence of the ADP-binding bab-fold in proteins using an amino acid sequence fingerprint. J Mol Biol 187:101-107
Woodfield D R, Matthew C (eds) Ryegrass endophyte: an essential New Zealand symbiosis. Napier, New Zealand, pp. 113-122
Yao Y, Peter A B, Baur R, Sigel E (1989) The tremorgen aflatrem is a positive allosteric modulator of the gamma-aminobutyric acid receptor channel in Xenopus oocytes. Mol Pharmacol 35:319-323
Young C A, McMillan L, Telfer E, Scott B (2001) Molecular cloning and genetic analysis of an indole-diterpene gene cluster from
Penicillium paxilli. Mol Microbiol 39:754-764
Young C, ltoh Y, Johnson R, Garthwaite I, Miles C O, Munday-Finch S C, Scott B (1998) Paxilline-negative mutants of Penicillium paxilli generated by heterologous and homologous plasmid integration. Curr Genet 33:368-377
Choreo and Goodman, Acc. Chem. REs., 1993 26 266-273
Creighton T. E. ‘Proteins Structure and Molecular Properties.’ W H Freeman and Co. 1984.
Freidinger, R. M., Perlow, D. S., Veber, D. F., J. Org. Chem. 1982, 59,104-109
Gallop, M. A., Barrett, R. W., Dower, W. J., Fodor, S. P. A. and Hogan, Jr., J. C. Nature Biotechnology,1997 15 328-330.
Krause, M. H. and S. A. Aaronson (1991) Methods in Enzymology, 200:546-556.
Nagai, U., Sato, K. Tetrahedron Lett. 1985, 26, 647-650.
Olson et al., J. Med. Chem., 1993 36 3039-3049.
Smythe, M. L., von ltzstein, M., J. Am. Chem. Soc. 1994, 116, 2725-2733

1. An isolated nucleic acid molecule having a nucleic acid sequence selected from the group consisting of:
a) SEQ ID NOs 1, 3, 5, 17, 19, 21, 7, 9, 11, 13, 15, 52, 54 and a combination of these sequences;
b) SEQ ID NOs 23, 24 and 25;
c) a functional fragment or variant of the sequences in a) or b); and
d) a complement to the sequences in a), b) or c).

2. An isolated nucleic acid molecule having at least 70% sequence homology to a nucleic acid as claimed in claim 1.

3. An isolated nucleic acid molecule having at least 80% sequence homology to a nucleic said as claimed in claim 1.

4. An isolated nucleic acid molecule having at least 90% sequence homology to a nucleic acid as claimed in claim 1.

5. An isolated nucleic acid molecule having at least 95% sequence homology to a nucleic acid as claimed in claim 1.

6. An isolated nucleic acid molecule having at least 99% sequence homology to a nucleic acid as claimed in claim 1.

7. An isolated polypeptide having an amino acid sequence selected from the group consisting of:
a) SEQ ID NOs 2, 4, 6, 8, 10, 12, 14, 16, 18, 20, 22, 53, 55 and a combination of these sequences; and
b) A functional fragment or variant of the sequences listed in a).

8. An isolated polypeptide molecule having at least 70% sequence homology to a polypeptide as claimed in claim 7.

9. An isolated polypeptide molecule having at least 80% sequence homology to a polypeptide as claimed in claim 7.

10. An isolated polypeptide molecule having at least 90% sequence homology to a polypeptide as claimed in claim 7.

11. An isolated polypeptide molecule having at least 95% sequence homology to a polypeptide as claimed in claim 7.

12. An isolated polypeptide molecule having at least 99% sequence homology to a polypeptide as claimed in claim 7.

13. A primer capable of specifically binding to a nucleic acid molecule selected from the group consisting of SEQ ID NO. 11 or SEQ ID NO. 12.

14. A primer having a nucleotide sequence selected from the group consisting of SEQ I) NOs 26-51.

15. A primer having a nucleotide sequence which comprises at least substantially 15-20 contiguous nucleotides of a nucleic acid molecule selected from the group consisting of: SEQ ID NOs. 1, 3, 5, 17, 19, 21, 7, 9, 11, 15, 15, 52 and 54.

16. A probe capable of specifically binding to a nucleic acid molecule as claimed in claim 1.

17. A method of identifying at least one gene of the lolitrem gene cluster in an endophyte comprising using a probe capable of specifically binding to a nucleic acid molecule as claimed in claim 1, thereby identifying said at least one gene of the lolitrem gene cluster in an endophyte.

18. An isolated nucleic acid molecule which is able to stringently hybridize to a nucleic acid molecule as claimed in claim 1.

19. An isolated nucleic acid molecule as claimed in claim 18 wherein the molecule is a primer.

20. An isolated nucleic acid molecule as claimed in claim 18 wherein the molecule is a probe.

21. A method for identifying mutations in the lolitrem gene cluster of an endophyte exhibiting useful phenotypic traits characterized by the steps of:
a) identifying at least one gene in the lolitrem gene cluster of an endophyte;
b) sequencing the gene(s) identified at a);
c) comparing the sequence at b) to SEQ ID NOs 1, 3, 5, 17, 19, 21, 7, 9, 11, 13, 15, 52 and 54 or a combination of these sequences to ascertain any differences in nucleotide sequence.

22. An endophyte in which at least one of the genes in the lolitrem gene cluster has been mutated or otherwise disrupted to manipulate the indole diterpene biosynthetic pathway.

23. The use of a nucleic acid molecule as claimed in claim 1 to produce an indole diterpene enzyme, intermediate or other chemical compound associated with the indole diterpene biosynthetic pathway.

24. (canceled)

25. A construct which includes a nucleic acid molecule as claimed in claim 1.

26. A host cell which includes a non-endogenous nucleic acid molecule as claimed in claim 1.

27. An endophyte which includes a non-endogenous nucleic acid molecule as claimed on claim 1.

28. A method of catalyzing in vitro or in vivo a reaction involved in the biosynthesis of an idole diterpene comprising adding a polypeptide as claimed in claim 7 to the reaction, thereby catalyzing said reaction.

29. A kit for identifying the lolitrem gene cluster which includes a probe or primer capable of specifically binding to a nucleic acid sequence selected from the group consisting of:
(a) SEQ ID NOS. 1, 3, 5, 7, 9, 11, 13, 15, 17, 19, 21, 23, 24, 25, 52 and 54; and
(b) A functional fragment or variant of the sequence in (a).

30. A kit for identifying the lolitrem gene cluster which specifically includes at least one primer pair selected from the group consisting of SEQ ID NOS. 26, 27, 28, 29, 30, 31, 32, 33, 34, 35, 36, 37, 38, 39, 40, 41, 42 ,43, 44, 45, 46, 47, 48, 49, 50, and 51.

31. A method of manipulating the indole diterpene biosynthetic pathway characterized by the step of altering a nucleic acid as claimed in claim 1 to produce a gene encoding a non-functional polypeptide.

32. A method of manipulating the indole diterpene biosynthetic pathway comprising using a gene produced by the method of claim 31, thereby manipulating said biosynthetic pathway.

33. An expression system which includes a non-endogenous nucleic acid molecule as claimed in claim 1.

34. A method of producing an indole diterpene, enzyme, intermediate or other chemical compound associated with the indole diterpene biosynthetic pathway comprising using an expression system as claimed in claim 33, thereby producing said indole diterpene, enzyme, intermediate or other chemical compound.

35. A method of specifically amplifying a nucleic acid molecule comprising using a primer as claimed in any one of claims 13-15, thereby specifically amplifying said nucleic acid molecule.

36. A plant including a cell which includes a non-endogenous nucleic acid molecule as claimed in claim 1.

37. (canceled)

38. (canceled)

39. (canceled)

40. (canceled)


SEQUENCE LISTING (55 sequences)

SEQ ID NO: 1 - DNA, 1110 bp, Neotyphodium lolii
atgacgatggctgccaatgactttccatttcaatgccaggagaagaaatcatattctcagccaagtctagtctactgcaatggtaacattgcggagacgtatctcgaagaaaaggtatttatactgctcctttataatctcgaatgccacttaaaatttagacaggttttgacagcgccgttggattatttgcgtgccttacctagcaaagatattcgcagtggactgaccgacgccattaatgagttcctgcgtgtcccagaggaaaaggttcttgtcataaagcgtataattgatcttcttcacaatgcatccttactgtaagttcgagattgcataacatagacctagtagattctaactaacagctttagcattgatgatatccaggattcatccaaactgcgacgtggagtccctgtagcccaccacatatttggaatcgcacaaacaataaattcggccaatctagcgtatttcattgcccagagagagcttgagaagcttacgaatcctcgagcatttgctatatataatgaggagctaatcaatctgcatcgtggtcagggtatggagctccattggagagaatcgctccattgccctaccgaagatgagtatctgcgaatgatccaaaagaagacaggcggtctgttccgattggcaatcagactgctgcaaggcgaaagcgctagcgatgacgattatgtctcacttattgatactctcggaaccctgttccagattcgagatgactatcaaaacttacagagtgatatatattctaagaacaaaggctactgtgaggatttaacagagggcaaattctcgtatccggtcatccatagtattcggtcgcgaccaggagatgttcgattaatcaatattttgaaacagcgtagtgaagatgttatggtgaagcaatacgcggtgcaacatatcgaatctacaggaagcttcgcattctgtcaaaataaaattcaatctttggtggagcaagcaagagagcaattggcggctctagaaaatagcagttcatgtggaggccccgttcgcgacatccttgacaagttagcaataaaaccacgggcaaatatagaagtagagtag

SEQ ID NO: 2 - protein, 334 aa, Neotyphodium lolii
MTMAANDFPFQCQEKKSYSQPSLVYCNGNIAETYLEEKVLTAPLDYLRALPSKDIRSGLTDAINEFLRVPEEKVLVIKRIIDLLHNASLLIDDIQDSSKLRRGVPVAHHIFGIAQTINSANLAYFIAQRELEKLTNPRAFAIYNEELINLHRGQGMELHWRESLHCPTEDEYLRMIQKKTGGLFRLAIRLLQGESASDDDYVSLIDTLGTLFQIRDDYQNLQSDIYSKNKGYCEDLTEGKFSYPVIHSIRSRPGDVRLINILKQRSEDVMVKQYAVQHIESTGSFAFCQNKIQSLVEQAREQLAALENSSSCGGPVRDILDKLAIKPRANIEVE

SEQ ID NO: 3 - DNA, 1647 bp, Neotyphodium lolii
atgactagcgacttcaaggtaataatcgtgggaggatcagtggctgggctttcactagcccactgcttagaaaaaatcggtgtttctttcatggttctagagaagggtaatcaaatagctccccaactcggtgcctcaattggcattttgccaaatggtggacgtattcttgatcaactgggcatcttccatagcatcgaggatgaaatcgaacctctagaatctgctatgatgagatacccggatggtttctctttcaaaagtcaatatccccaagctttgcatactaggtaataacagtgaaagaagagtggcctataagtgttcatatatcgctaacttcgtgcggttaatagttttggttatcccgtggctttccttgagaggcaaaggtttcttcagatactttatgataaactcaagagcaaagactgcgtttttacaaacaagcgggtagtcagtattgcaagtggccaagacaaagtcacagcaaagacttcagatggcgctaagtacttagcagatatcgtgatcggtgctgacggggtccacagcatcgtcaggtcagagatttggaggcatttgaaggaaaactctcaaatatcagtattagaggcaccgaacgcaagtaggttaacctaggattaattgcaaagaaactttactaatgagggagccacttaggtattaagcatgattattcatgcatttacggaatttctttaaacgttccccagatcatcctaggaatacagttaaactgtttagatgacggagtgtcaatacacttgtttacgggtaaacaatccaaattattttggtttgttatcatcaaaacgcctcaggctagctttgctaaagtagagattgacaatacacatacagcaaggtgtatctgcgaaggactgaggacgaaaaaggtttcagataccttatgttttgaagatgtatggtcaagatgcaccatattcaagatgacgcctcttgaggaaggggtgtttaagcattggaactatggccgcttagcatgtattggtgatgctatccgcaaggtatgtggatgatgctatatgtccctatttcgtgtcatcagtgggatgacaaaagaaggccactatttgccgctaatataaatgatcgtatcgctaacattaacagatggccccaaataatgggcaaggagcaaatatggcgatagaggacgcttgcagtctcgcaaacatcctccagaaaaagatatcacatggttcgattcgagaccaagatatcaattcaatgtttcaggaattctctatggctcaacgggctcgcacggagagcgtctgcgcgcagtcggagtttctagtccgcatgcatgcgaatcaaggtattggaagaagacttcttgggcggtaccttattcctttcctgtatgacgcacctgctggtttatctggattttctataagtggcgcaacaagaatagagttcatagacttgcccactagatctcttaggggagcgtggggaaagtcatggagagggtcatgggaattcatcctacaaagcttggtctatttgcgacccaagtttaggatagtttatgccttgtatctcgttgcagctgcagcttttatcttgtattgtcttagcagtctcttcccgtag

SEQ ID NO: 4 - protein, 472 aa, Neotyphodium lolii
MTSDFKVIIVGGSVAGLSLAHCLEKIGVSFMVLEKGNQIAPQLGASIGILPNGGRILDQLGIFHSIEDEIEPLESAMMRYPDGFSFKSQYPQALHTSFGYPVAFLERQRFLQILYDKLKSKDCVFTNKRVVSIASGQDKVTAKTSDGAKYLADIVIGADGVHSIVRSEIWRHLKENSQISVLEAPNASIKHDYSCIYGISLNVPQIILGIQLNCLDDGVSIHLFTGKQSKLFWFVIIKTPQASFAKVEIDNTHTARCICEGLRTKKVSDTLCFEDVWSRCTIFKMTPLEEGVFKHWNYGRLACIGDAIRKMAPNNGQGANMAIEDACSLANILQKKISHGSIRDQDINSMFQEFSMAQRARTESVCAQSEFLVRMHANQGIGRRLLGRYLIPFLYDAPAGLSGFSISGATRIEFIDLPTRSLRGAWGKSWRGSWEFILQSLVYLRPKFRIVYALYLVAAAAFILYCLSSLFP

SEQ ID NO: 5 - DNA, 2063 bp, Neotyphodium lolii
atgcaatacggtaatttaacaactgtattacttctgcgtaatactttattgtccttgaattcttcgtcaatctgccatgttcactggctgcaagtgattgtggctctgcttgtcttgatcgtctgcatctttctatattggcgaacacccactggcatcaatgctcctttcgcaggatatcgttcaccatgggagccgccgctcttggttcagatgcgttacgtcttcaacgctgcctcaatgatacgcgaaggatatgctaaggtatgttttatcccgcgtagaggtcttctacccggatagaccgagaagataacaacttcggaacagtggaaagactccttgttccagatctcacgatacgacggtgacattcttattgtgcctccaagatatttggatgacctccacaacaagtcacaagaggagttaagtgctatttatggtttgattcgggtgaggaatgccaccaaccaaaaaacgcagagcctattagcgcatggtctcacatattcgaatttgctagaattttggtggtagctatagcggcatcaccctgcttggagaaaacgatgttggcattcgtgcgcttcaggtatgtacacccttccaaaagtctgttagggaccttccttactctactacagacaaaaatcaccccaaatcttgcgaaattatgcgatgacataagggatgagtttcagtattgtctagatacagacttcccagcctgcagaggtatgccatttccaaaatcccattatgcagtctctactttttctggcactaacgatatctaacatagattggacatcagtgtccgtgcatccattgtttctaaaagcagtcgaaaggataacacatcggatttttgttggattgccattatgtcggaatccccaatgggtccaagcgaccagcaagcatgcacattacggtacgtcaattgactaataataggcaatatacgcgctcatatgctttgcagcaacaatgatacagatagctatgagatctgtcccaaagttcattcagcctttactaaatttttgccttccgtggccatggaagaacgcagcctgtgttcgtgaagcaaagaatgcccttatattagaaatgcaacgccgacgaaatctcgagaaagttaacagttttgattatatcaaatccaatgacttgctgcaagcagttatggaaatgtcttctcctagtcatgaggatagccagcttgatgttgtcgcccagataatgctcacgatgaacacaatcgctggccacagtactgccgcatccggagcacatgcactgttcgatatggttagccactctaagtatattgaattgctgcgtgaggaggctcttcaagtctttcgacatgttgaactgcgtgttacaaaacaggctttgggggatttgcgaaaattggacagcttcctcagagagttagtattgtcctaaacatcacaatctcaccacattctcacgctagcttttcctccgtactaatgatggtcgttgctaagatcccaacgacataatccgctaagcttgtgtatgtttagctaagagtctcgaaaacctggaaatgtttgtcctgtgcccgagttctaacgtctcttactacagtaggcttttttcgggtcgtattagaccctgccggtatcacacttcaagatggcacacatgttccttacaacacactgctttgtgtcgcaccacatgcgatatccaatgacccggatgtgatagaagacccaacctcgttcaacggtctgcgatactacgaacagcgctgtcgtgacgccagtcaagagaaaaagcatcaatacgctactacggataaatctcacctgcattttggctacggaacctgggcctgtccaggccgcttcttggcctctgatatgttaaaagtgattctaacgatgcttctgcttcagtatgacatccgctcccccgagagagcaaaacggcctgtggcaggtcattttcatgagtttccgcttttcaatattaacacaccactgttaatgaaacgacgcaatgattcgctagttctatga

SEQ ID NO: 6 - protein, 533 aa, Neotyphodium lolii
MQYGNLTTVLLLRNTLLSLNSSSICHVHWLQVIVALLVLIVCIFLYWRTPTGINAPFAGYRSPWEPPLLVQMRYVFNAASMIREGYAKWKDSLFQISRYDGDILIVPPRYLDDLHNKSQEELSAIYGLIRNFGGSYSGITLLGENDVGIRALQTKITPNLAKLCDDIRDEFQYCLDTDFPACRDWTSVSVHPLFLKAVERITHRIFVGLPLCRNPQWVQATSKHAHYATMIQIAMRSVPKFIQPLLNFCLPWPWKNAACVREAKNALILEMQRRRNLEKVNSFDYIKSNDLLQAVMEMSSPSHEDSQLDVVAQIMLTMNTIAGHSTAASGAHALFDMVSHSKYIELLREEALQVFRHVELRVTKQALGDLRKLDSFLRESQRHNPLSLLGFFRVVLDPAGITLQDGTHVPYNTLLCVAPHAISNDPDVIEDPTSFNGLRYYEQRCRDASQEKKHQYATTDKSHLHFGYGTWACPGRFLASDMLKVILTMLLLQYDIRSPERAKRPVAGHFHEFPLFNINTPLLMKRRNDSLVL

SEQ ID NO: 7 - DNA, 1115 bp, Neotyphodium lolii
atgacatctggagcatggctcgtggctcgccctgcggccatcgaaattgcggccctcttgtttgcatttacgctcgggtatctagtaaagtacacaatcaattaccaatctgtcgtttctcaagccattgatcattatggctatggctatgaacgtacctctcacgaaggtattggcggcagcaatggcaagattcctgactgtccatactcttatgtgattagtctctatgggcataatcatttctctcccctcgtggattttcttcatccaacattgaaacataaatatcccaagaaacattctttgatcctggatatcatggatgcggtccatctttgtctaattatggttgacgatatttgcgaccacagccctaagcggaaaaatcacactacggctcacttgctatacggatcatgcgaaactgccaatcgagcatacttcgttctcacaaaggtcattaatagagcaatgaaagaacaacctgtccttggaattgaacttctgagagcactagaactgatactcgagggacaagacatgtctttggtttggcgaagagacggtttgcgatctttcgaatcctatggtgaagaaagcctgttgacgtacaaaaatatggctctgctgaagacaggcacactttttgtgctccttgggaggcttttgaaccaaggaggtcatcaatcagacgatctgttaggccgatttgggtatgtaaatttttttttttttcgctcgtttcataattccgcggcaaggtcgcttaactaattcaatggaatggtagctggtacgcacaattgcaaaatgattgcaagaacatatactcagaagagtacgcttttaacaaaggcactgttgcagaagacctacgcaacagagaattgtcctttcctgttgtggttgctcttaatgacaaacatactgagccgcagataaggaaggcgtttcagagccaaaatcaaggcgacattaaacgggcactccaagcgttagagtcacctagtgttaaaaacacgtgtctcaaaacgctccaggaggcaggtcagggtctagagaacttggtggccgtctggggacgaaaagaacaaatgcactttacaaaatga

SEQ ID NO: 8 - protein, 345 aa, Neotyphodium lolii
MTSGAWLVARPAAIEIAALLFAFTLGYLVKYTINYQSVVSQAIDHYGYGYERTSHEGIGGSNGKIPDCPYSYVISLYGHNHFSPLVDFLHPTLKHKYPKKHSLILDIMDAVHLCLIMVDDICDHSPKRKNHTTAHLLYGSCETANRAYFVLTKVINRAMKEQPVLGIELLRALELILEGQDMSLVWRRDGLRSFESYGEESLLTYKNMALLKTGTLFVLLGRLLNQGGHQSDDLLGRFGWYAQLQNDCKNIYSEEYAFNKGTVAEDLRNRELSFPVVVALNDKHTEPQIRKAFQSQNQGDIKRALQALESPSVKNTCLKTLQEAGQGLENLVAVWGRKEQMHFTK

SEQ ID NO: 9 - DNA, 1829 bp, Neotyphodium lolii
atgttaatgttgcacgctgtcccagtgggtatctgtttactactgtggtacgttgtttacggtaccaaacggaaagaatgtataccaaccattcgacgttggcctcgattactcccccaattcctcgatcggctgagttataatgaccatgccgcccgcctagtcaaacatggctatgagaaggtgttcggtcccaagtctgtgagcatccgtgatgatagtactaaccgcctaggttacagcacaaaaatcaaccgtttaggctacttaagatggacatggatctgattgtcattcctttacaatacgcgctggaattacgggcggttacgagcgacaaattagaccctttaacagccagctttgatgacaatgctggtaaagttacgaggatattattagggagcgaacttcacacacgtgccatacagcagcgtttgactccaaagcttcgtaagtgtaatctaacagaatacgtttgcactatgctaactgaatccagcacaaactcttccagtgctattggatgagctcaatcatgcctttgggcaagtcttacctgccggcaacgacggtatgtgttttcattctttcaaacattccacttgtctagtgttgtctaatattagtggaggttccaatgcttggatttctgtcaatccatacgaattggttctcaatctagctacccgtgctacagcgaggctattcgttggagacctgatttgtcgaaacgaaatttttctcgagactactgcttcttttagtcgcaacacgtttgatacgatatccacctcccgtagttttggcaatttgttcacacattatttcgcacggtggatttccacagcgaaagaagctcacgggcaattacaatacattcaaaacctccttggttcagaagtccagagaaggaaacttaactctgaggaaaagcacgacgactttttgcagtggtgtacagagttagcagtcaccgaggatgaagcacggccagaagcacttgcgcatcgtacgctaggaatattgagcatggctgtcattcatacaacagctatggcgttaactcacatactttttgacatgatctcggacgacagcttgaaggagagcctccgaagagaacagcaaaacgtgctcaagcatggttggacggaaatcacgcaacagactatgcttgatatgaaacaattggatagcctgatgagagagtcacaacgaatcaatccagtgggcgagtgtaaggactattctaagccagtcttttgtcaaacttggaactaactggcgattgaagtcacttttagacgcattgtccgagaacgaattacattgtccgatggctaccagctacagccgggacagcagattgcgattccagcgaagtgtatcaatacggacagtacgaaattatccgacgctcacttgtttcaaccttttcgatggttgaaacaatctggcactgccacaacatcattttctaacagcagcgccttgaatctgcacttcggatttgggagatatgcctgtccgggacgcttcatagcttccgtatgtgatgtagattttcatcttttttttttccatatcaatctcccttcaagctcatgtgacgcacattcgaccttcttgactaacccttgagtttgtgctcatagtatatgattaaagcaatcatgagtcggattctgctcgagtatgattttaagctagatagtgagtttccgtcgcggcgccctcctaacattgttcatggggataagatcctccccaatcggaatgccgttgttcttttgcgccgcttggagaagacagttaccgtatgttga

SEQ ID NO: 10 - protein, 498 aa, Neotyphodium lolii
MLMLHAVPVGICLLLWYVVYGTKRKECIPTIRRWPRLLPQFLDRLSYNDHAARLVKHGYEKHKNQPFRLLKMDMDLIVIPLQYALELRAVTSDKLDPLTASFDDNAGKVTRILLGSELHTRAIQQRLTPKLPQTLPVLLDELNHAFGQVLPAGNDGSNAWISVNPYELVLNLATRATARLFVGDLICRNEIFLETTASFSRNTFDTISTSRSFGNLFTHYFARWISTAKEAHGQLQYIQNLLGSEVQRRKLNSEEKHDDFLQWCTELAVTEDEARPEALAHRTLGILSMAVIHTTAMALTHILFDMISDDSLKESLRREQQNVLKHGWTEITQQTMLDMKQLDSLMRESQRINPVGEFTFRRIVRERITLSDGYQLQPGQQIAIPAKCINTDSTKLSDAHLFQPFRWLKQSGTATTSFSNSSALNLHFGFGRYACPGRFIASYMIKAIMSRILLEYDFKLDSEFPSRRPPNIVHGDKILPNRNAVVLLRRLEKTVTVC

SEQ ID NO: 11 - DNA, 1945 bp, Neotyphodium lolii
atggcatttgcaagtcttttgcaccatatctggaaccatgcagtggattgcgctgagcagctgacttggtggcagaccattgtgagcttcatcattttctgcatcatgtgctcttggctacctgggaatggggaaatgcgcgctccgtttgttggttatcgctggccattcgagcctactttctgggtccgaatgcgcttcatctttcagagtttaggcatgatgaccgaaggatactcaaaggtgagctcccgtccgggtggagaaagacagctagacgaatgactgacgccaaacgcttgacagttcaaggattccatgttcaagatcacgaccaacgatgccgactggcttgtcctctcccaacgctacttggatgacttgcagtctctgccagccgagagattgagccatacagacgctctagtgacggtgagggcgcatactagtcgctagtccctacgacagtggtgtgctaatcgagttgtgtctcatttagatgtgggggagcagccacagcccttttgctctgctcaacaagagtgatcttagctctcgagctcttcgtgtaaggaccaatccctccttgttatgcagaacggatctgacttgaaaaggacgtggttgcgccgaattatgccaaggaccttgatagcctcgtagacgaactccgctattcgcttgagcacgatatagacatacaggatggtatgtatgcgcctattttccaactaattttgaggtcgtcatgttggctgactgggtcgatgcgcttagactggaaaccgattgatgcccttgaactttcttcgaagttggtgttgcggatatcgcagcgaatcttgatcggctggcccatgagtcgcgatcaagagctccttgaatgcgcacaaggctacgcagacgctggtaagaggacgagctgttacgtatgacccttttcttcggtaaaaactaacgggggtttcagctaccgtcgtccagtttgccctgaaactacttcctcgccagattcggccgcttgtctatcctctgctcccacaagcatgggctactaaatcgtggatcaggcgctgtgacaagatactggcaaaggaaatgcaacgtcgacaagttttggagaagtcggatcccgtgtacgagaaaccaaaggacttgctgcagggcatggtggacctggagccgtcccggcctgttgacaaacttggacatgattttctcgtccaagccttgatttccagaatggctccagttgttaccatggcccaaacccttgttgatcttgccctccatcctgaggatatcgaggagctgcgtgatgaggttctgcaagtcataggaccagacggggcgggattaggaaacctacgacaatcatttaccaaacttgacaagatggacagcgtcttgagggaatctgccaggttcacccctctatctatgagtaagtgccatttctgtcctccagaatagcttgctggcatgactaatctgtggtatagtgacaatgcaccgccgggttcaggacgccaagggcatcacgctccatgacggtgtgcatcttccacgaggcacgcatgtggcattcccagcgtaccacattggcagagatcccaagttggtgtcaggtgcagatatctatgacgggctgcgctggtacaggaaggacctcggcgaggcccaagaaaacgaagctcccaagcatcgatttgtcacccccgacagcaactacttgacctttgggtccggtaaatacgtctgccccggccgatttatagcggaacacatgttgaagctgatgatgaccgccgtgctcctgcgctacgagttcaagtggcctccgggagtccctgtgcccgaacaacagtatcggcatgtctttgcttatccaagcaaaaccacactgttgattaaacgacgcaaagatggcgatcagattctttaa

SEQ ID NO: 12 - protein, 525 aa, Neotyphodium lolii
MAFASLLHHIWNHAVDCAEQLTWWQTIVSFIIFCIMCSWLPGNGEMRAPFVGYRWPFEPTFWVRMRFIFQSLGMMTEGYSKFKDSMFKITTNDADWLVLSQRYLDDLQSLPAERLSHTDALVTMWGSSHSPFALLNKSDLSSRALRDVVAPNYAKDLDSLVDELRYSLEHDIDIQDDWKPIDALELSSKLVLRISQRILIGWPMSRDQELLECAQGYADAATVVQFALKLLPRQIRPLVYPLLPQAWATKSWIRRCDKILAKEMQRRQVLEKSDPVYEKPKDLLQGMVDLEPSRPVDKLGHDFLVQALISRMAPVVTMAQTLVDLALHPEDIEELRDEVLQVIGPDGAGLGNLRQSFTKLDKMDSVLRESARFTPLSMMTMHRRVQDAKGITLHDGVHLPRGTHVAFPAYHIGRDPKLVSGADIYDGLRWYRKDLGEAQENEAPKHRFVTPDSNYLTFGSGKYVCPGRFIAEHMLKLMMTAVLLRYEFKWPPGVPVPEQQYRHVFAYPSKTTLLIKRRKDGDQIL

SEQ ID NO: 13 - DNA, 2014 bp, Neotyphodium lolii
atgaagatgttaacagagcattttgactttcctaaacttaacttcgccaccattgtaatttcaggcgccaccattattggtataatattccttcgatatcttaattaccctacaaaggtttgaatctgtttcccttgctatgcacttttcttggatgctcaccaaaatttttcaaggtgaatgtacctgttgttggcattggagttcgatatacaaaatggctagcggctattataaacgtgcgtcatgctcgacaatctatccgcgagggctatgcaaaggtttgtgttaaaaacgaataaaagcgcttcgtaaacaaagagaactaatactagtttctagtatggcgatttcgcgtttcagatacctactatgactcgaatggaggtattcatttgtgatagacagatgacaagggagtatcagaatgttgacgactatcatttgtcgttccgagctgtcatgaccgaggtaagtaactagaccatgttaactgtaggaaaagaagaaaaagctaaaccgccgtacaggagtttcaattcaaatggctacttccaggacaggcacacgaagcccggattatccctaactcagtgattgctaaggccttgagctggcagagaacaagggcgaataaacccagcgatccattcttcgaatctttctccgccgaattcatgcaggggtttcaggaagagatgcgacgactaatccaatatcaaaattcgtcagttatgtcaaaccgctccggtgctgtcctggatccagcgcatggttggcatgctgtgccttgttttcccttggctctgaaggtaattgggcgccttactacatacgtcttgttcggcaaacctttgtgccaagatgcgacattcctaaacatgtgctgtcaatttggcgatgtgattcccagggatgcgatcatactacgttcatggccagcattggcaaggccgtaagcaagtgcctagacataaacccgtcagggtttaaactcgcattaacattcatatagtcttattgtaaagatcttgagtgctccaagggttatgggaaagttgcgaaacattttgattgttgagataaagagcaggagagaatcccacgaaacgaacccaatgagtgtatggctgtctcgcacaccccctctagcattacacattaacgtatatctaggatatcttggatttcacaatggcctgggttgaccgtcatcctaacgctagctttgacgatcagcacattgccgagatgatgattaacactattttcgcagctcttcatacgtcgagtcaggtatatttttttctgtatgaaaagtccagagcttaaagctaactggctcatagctggtggtgcataccatctttgagcttgcctcacgtcctgaatatagcgatgcgcttctggaagagatagatgcatgctttgaaaagcatggaaagggcactaaagcagctctagactcaatgttcaaggtggatagtttcatcaaagaaacgcagaggtttaaccctcttgacgcatgtataaattccctgtctccgattccatcattgcgatttgactaacgccaccgtcagccgctcttgcaagactggctctcaaagactttactttttccaatggcctaaacatcccaaagggcagtgtgattttcacgccgaattcgcctatctttgaggacgagagatattacaaggatccgaaagtttttgatggatttcggtttgctaggatgcgtaatgacccaaaattaggtctattctgcgacctaacagcaacgaatgaacaaagcatgcattttgggactggacgtcacgcctgtcctggtagatttatggtttctgatgaggtcaagttagctgtgattcatatcttaagtaatttcgatttttgtattgagaattttggaccacggccagcaaatcagccatttggtaaatttcttctacctgatatgagtgcaaaaatctggctaagggagaaaagagctagggagaagaatctgtga

SEQ ID NO: 14 - protein, 537 aa, Neotyphodium lolii
MKMLTEHFDFPKLNFATIVISGATIIGIIFLRYLNYPTKVNVPVVGIGVRYTKWLAAIINVRHARQSIREGYAKYGDFAFQIPTMTRMEVFICDRQMTREYQNVDDYHLSFRAVMTEEFQFKWLLPGQAHEARIIPNSVIAKALSWQRTRANKPSDPFFESFSAEFMQGFQEEMRRLIQYQNSSVMSNRSGAVLDPAHGWHAVPCFPLALKVIGRLTTYVLFGKPLCQDATFLNMCCQFGDVIPRDAIILRSWPALARPLIVKILSAPRVMGKLRNILIVEIKSRRESHETNPMSDILDFTMAWVDRHPNASFDDQHIAEMMINTIFAALHTSSQLVVHTIFELASRPEYSDALLEEIDACFEKHGKGTKAALDSMFKVDSFIKETQRFNPLDASALARLALKDFTFSNGLNIPKGSVIFTPNSPIFEDERYYKDPKVFDGFRFARMRNDPKLGLFCDLTATNEQSMHFGTGRHACPGRFMVSDEVKLAVIHILSNFDFCIENFGPRPANQPFGKFLLPDMSAKIWLREKRAREKNL

SEQ ID NO: 15 - DNA, 1496 bp, Neotyphodium lolii
atgattgcgaaaaatattgaactcaatggcttggatccggcaaccagggcattggacattctatactggaaaaatcactgcatcaaacagctagaatctctcctatgcgccacagattcatactgcactgcagacaaggccgctcaactacgcattttgtcagagttggtgctccccaatcttggccctcggccgtccaatgccactgggccatcctatcttacacgaagtggttccccaataatgttaagtctaaatacaacatcatcaaaaaactgcgtcagatattgctgggagattctaggggcgactggcgcaagtaatgatgatcctttggcagtccaagttgctaaggatgtagtggcttctctgtctgctacttttcgcctttcaacaaaatggagcgaaactctactgtccaattttgcagtaacaccagaccaagctcgacaagttattaacatgctacccgagtggattcaaggcttcgtacctgagggaatggagtgcgattttccaaagagaatcccgttcgccatgacatcattcgacctaaatggctccaatgtagctatgaagctctacgttaatccaagggtaaaggagattttaactggtactccctcatcagacttggtctgggagttcctccgaaatttaacaccagaaatgaaaccacgagcggtcgacttgcttgagaggtaagaatggctttgaactttcgcccaccttgtcagccccatacgctaagcgctaactccccacacattaacaggtttattaccgataattcaggcccgtctgctattgagcttgtaggtattgactgcgttgacgacgctcacctatcaaatgcaagggtcaagctttacgttcataccatgagcagctcatttaacaccgtaaagaattatgttactcttgggggtgcaatctgggatgaacaaacccaaaagggcttaggaatactacaaagtatttggcacctattgcttcaggagccagagggtatttctgacaatggattcgacaagcctgtgaacgactcttccatgttatgccaaaagctatattttagtttcgagctacgcccaggtacagacttccctcaggtgaagacctatgtgccaacttggaactatcttcgaaccgacggggaaactatccagaactatgaggcgatcttccgagcttgtgaccatccttggggtgaagataggacgtacggcaaaatttttcaagatgcattgtaagttatcccttcagattagcgctaaaaggagtttgagatactcctcaatgcaagctattaggttgtgaaattgccactactaattggagctttttatagcggacctgcaaccgagagtcggaaaaaacccattcactgcgacgcatcttttctgtttaccgaagaaactggtgtctaccagacgctgtatttcagtcctccgattgagggggaaacagaagtccagtcaaatctcgttgcttga

SEQ ID NO: 16 - protein, 439 aa, Neotyphodium lolii
MIAKNIELNGLDPATRALDILYWKNHCIKQLESLLCATDSYCTADKAAQLRILSELVLPNLGPRPSNATGPSYLTRSGSPIMLSLNTTSSKNCVRYCWEILGATGASNDDPLAVQVAKDVVASLSATFRLSTKWSETLLSNFAVTPDQARQVINMLPEWIQGFVPEGMECDFPKRIPFAMTSFDLNGSNVAMKLYVNPRVKEILTGTPSSDLVWEFLRNLTPEMKPRAVDLLERFITDNSGPSAIELVGIDCVDDAHLSNARVKLYVHTMSSSFNTVKNYVTLGGAIWDEQTQKGLGILQSIWHLLLQEPEGISDNGFDKPVNDSSMLCQKLYFSFELRPGTDFPQVKTYVPTWNYLRTDGETIQNYEAIFRACDHPWGEDRTYGKIFQDAFGPATESRKKPIHCDASFLFTEETGVYQTLYFSPPIEGETEVQSNLVA

SEQ ID NO: 17 - DNA, 1110 bp, Epichloe festucae
atgacgatggctgccaatgactttccatttcaatgccaggagaagaaatcatattctcagccaagtctagtctactgcaatggtaacattgcggagacgtatctcgaagaaaaggtatttatactgctcctttataatctcgaatgccacttaaaatttagacaggttttgacagcgccgttggattatttgcgtgccttacctagcaaagatattcgcagtggactgaccgacgccattaatgagttcctgcgtgtcccagaggaaaaggttcttgtcataaagcgtataattgatcttcttcacaatgcatccttactgtaagttcgagattgcataacatagacctagtagattctaactaacagctttagcattgatgatatccaggattcatctaaactgcgacgtggagtccctgtagcccaccacatatttggaatcgcacaaacaataaattcggccaatctagcgtatttcattgcccagagagagcttgagaagcttacgaatcctcgagcatttgctatatataatgaggagctaatcaatctgcatcgtggtcagggtatggagctccattggagagaatcgctccattgccctaccgaagatgagtatctgcgaatgatccaaaagaagacaggcggtctgttccgattggcaatcagactgctgcaaggcgaaagcgctagcgatgacgattatgtctcacttattgatactctcggaaccctgttccagattcgagatgactatcaaaacttacagagtgatatatattctaagaacaaaggctactgtgaggatttaacagagggcaaattctcgtatccggtcatccatagtattcggtcgcgaccaggagatgttcgattaatcaatattttgaaacagcgtagtgaagatgttatggtgaagcaatacgcggtgcaacatatcgaatctacaggaagcttcgcattctgtcaaaataaaattcaatctttggtggagcaagcaagagagcaattggcggctctagaaaatagcagttcatgtggaggccccgttcgcgacatccttgacaagttagcaataaaaccacgggcaaatatagaagtagagtag

SEQ ID NO: 18 - protein, 334 aa, Epichloe festucae; identical to SEQ ID NO: 2

SEQ ID NO: 19 - DNA, 1647 bp, Epichloe festucae
atgactagcgacttcaaggtaataatcgtgggaggatcagtggctgggctttcactagcccactgcttagaaaaaatcggtgtttctttcgtggttctagagaagggtaatcaaatagctccccaactcggtgcctcaattggcattttgccaaatggtggacgtattcttgatcaactgggcatcttccatagcatcgaggatgaaatcgaacctctagaatctgctatgatgagatacccggatggcttctctttcaaaagtcaatatccccaagctttgcatactaggtaataacagtgaaagaagagtggcctataagtgttcatatatcgctaacttcgtgcggttaatagttttggttatcccgtggctttccttgagaggcaaaggtttcttcagatactttatgataaactcaagagcaaagactgcgtttttacaaacaagcgggtagtcagtattgcaagtggccaagacaaagtcacagcaaagacttcagatggcgctaagtacttagcagatatcgtgatcggtgctgacggggtccacagcatcgtcaggtcagagatttggaggcatttgaaggaaaactctcaaatatcagtattagaggcaccgaacgcaagtaggttaacctaggattaattgcaaagaaactttactaatgagggagccacttaggtattaagcatgattattcatgcatttacggaatttctttaaacgttccccagatcatcctaggaatacagttaaactgtttagatgacggagtgtcaatacacttgtttacgggtaaacaatccaaattattttggtttgttatcatcaaaacgcctcaggctagctttgctaaagtagagattgacaatacacatacagcaaggtgtatctgcgaaggactgaggacgaaaaaggtttcagataccttatgttttgaagatgtatggtcaagatgcaccatattcaagatgacgcctcttgaggaaggggtgtttaagcattggaactatggccgcttagcatgtattggtgatgctatccgcaaggtatgtggatgatgctatatgtccctatttcgtgtcatcagtgggatgacaaaagaaggccactatttgccgctaatataaatgatcgtatcgctaacattaacagatggccccaaataatgggcaaggagcaaatatggcgatagaggacgcttgcagtctcgcaaacatcctccagaaaaagatatcacatggttcgattcgagaccaagatatcaattcaatgtttcaggaattctctatggctcaacgggctcgcacggagagcgtctgcgcgcagtcggagtttctagtccgcatgcatgcgaatcaaggtattggaagaagacttcttgggcggtaccttattcctttcctgtatgacgcacctgctggtttatctggattttctataagtggcgcaacaagaatagagttcatagacttgcccactagatctcttaggggagcgtggggaaagtcatggagagggtcatgggaattcatcctacaaagcttggtctatttgcgacccaagtttaggatagtttatgccttgtatctcgttgcagctgcagcttttatcttgtattgtcttagcagtctcttcccgtag

SEQ ID NO: 20 - protein, 472 aa, Epichloe festucae
MTSDFKVIIVGGSVAGLSLAHCLEKIGVSFVVLEKGNQIAPQLGASIGILPNGGRILDQLGIFHSIEDEIEPLESAMMRYPDGFSFKSQYPQALHTSFGYPVAFLERQRFLQILYDKLKSKDCVFTNKRVVSIASGQDKVTAKTSDGAKYLADIVIGADGVHSIVRSEIWRHLKENSQISVLEAPNASIKHDYSCIYGISLNVPQIILGIQLNCLDDGVSIHLFTGKQSKLFWFVIIKTPQASFAKVEIDNTHTARCICEGLRTKKVSDTLCFEDVWSRCTIFKMTPLEEGVFKHWNYGRLACIGDAIRKMAPNNGQGANMAIEDACSLANILQKKISHGSIRDQDINSMFQEFSMAQRARTESVCAQSEFLVRMHANQGIGRRLLGRYLIPFLYDAPAGLSGFSISGATRIEFIDLPTRSLRGAWGKSWRGSWEFILQSLVYLRPKFRIVYALYLVAAAAFILYCLSSLFP

SEQ ID NO: 21 - DNA, 2063 bp, Epichloe festucae; identical to SEQ ID NO: 5

SEQ ID NO: 22 - protein, 533 aa, Epichloe festucae; identical to SEQ ID NO: 6

SEQ ID NO: 23 - DNA, 11400 bp, Neotyphodium lolii
aatggactagaaagtacatttgttatacagtgctatctccttaggctcagtctaccttgtgggtcagtgcaggccccacaggccccctgccacaaggttagtaaccgcgcaagcacgcgaaagtgtagcgtagtaaattatataggaaaaattagcagtatattaattattagcctatctatatataagtaaatatacctttaattcacttctatttaattggatatagaccctagttaacgtgacttcacaaggtgaactaagtccaagaagatagaggtaattgcagtgagatccacaggtcttgtcaggggacggcaatgtatgcatatatcgtgaaatcaatgctagcggcattgaatcaatgacttctgtagctagcgataatagcagcgatagaagcctctagaatctatatagacagtattaagtaaactctccacctgtatccacagctaacttacatacacctagccctgtcttgagtgcttttagaagactatgctaacttagatcacaccctaagtgccaatgtctcccaattagccgcgaagagagaacttatcgcaaggaagtgataaggctataacatccaacaggttacttaaagacaacaggctaggaatcaattatagtagcaatcaaaactagatcctgtattctataacaagaagttaaatcccccctagactatctgtctatctttagttatactttggttttgctttgttgtcttatgcctacattcctaaaagatctttatgacgatggctgccaatgactttccatttcaatgccaggagaagaaatcatattctcagccaagtctagtctactgcaatggtaacattgcggagacgtatctcgaagaaaaggtatttatactgctcctttataatctcgaatgccacttaaaatttagacaggttttgacagcgccgttggattatttgcgtgccttacctagcaaagatattcgcagtggactgaccgacgccattaatgagttcctgcgtgtcccagaggaaaaggttcttgtcataaagcgtataattgatcttcttcacaatgcatccttactgtaagttcgagattgcataacatagacctagtagattctaactaacagctttagcattgatgatatccaggattcatccaaactgcgacgtggagtccctgtagcccaccacatatttggaatcgcacaaacaataaattcggccaatctagcgtatttcattgcccagagagagcttgagaagcttacgaatcctcgagcatttgctatatataatgaggagctaatcaatctgcatcgtggtcagggtatggagctccattggagagaatcgctccattgccctaccgaagatgagtatctgcgaatgatccaaaagaagacaggcggtctgttccgattggcaatcagactgctgcaaggcgaaagcgctagcgatgacgattatgtctcacttattgatactctcggaaccctgttccagattcgagatgactatcaaaacttacagagtgatatatattctaagaacaaaggctactgtgaggatttaacagagggcaaattctcgtatccggtcatccatagtattcggtcgcgaccaggagatgttcgattaatcaatattttgaaacagcgtagtgaagatgttatggtgaagcaatacgcggtgcaacatatcgaatctacaggaagcttcgcattctgtcaaaataaaattcaatctttggtggagcaagcaagagagcaattggcggctctagaaaatagcagttcatgtggaggccccgttcgcgacatccttgacaagttagcaataaaaccacgggcaaatatagaagtagagtagttgacattaagaacattgcgataaaagacacttttactatactcgactagttttaaaactatgtgtgagattaagacgtcttcaggtactcaaagtgtggaagtatgtcacgcagaaaagagctaacattgctctcagcttcctcactatttagtttcaccaagagcatccttcatagagacatttgcggctgtgattttcgtttacgtcatgttgttaaacattgttgtatggtatctttgcttaggagtagacatccattttctctcactctactcttagagatcgtcaagtgtcacatacatttctgagaactaggactttgcatagaatatgcatcggttaggtgtttgcgtagagagtacgtgtgtctgaggttagccattgcgcttcgtttgcggtttacaatggggcaaggcttaaagctttttaaagccacggtgaccactactgcaggtgcattctttttttagtcgtaaaacactaagttttttttactagttataatagacttttctttccttcttcccttctcgtagataaacccaattgaagaattaatataaagtgtattcttaatcctagccttatccctaaatatatatatatatattgtatactctagctagctctatgtagggctagttctagtactgcctctagttagttaaaagggaaaacccttaaataagaagaaaaatccctttatattttgtcaggcgaaaacaaccacccgaaaacgacggatttgacgatgacactaacaacaaagctaacgaatttgacgatattagcaattgaacctagatatcgggatctaggtctgcgaggtttccgatccacgcctaggattcaagctagggggtagggtctttttctaataataggttattttattaattaaacaatccaagcctaaggcaacgaagggagagtaaagtttctatttaaagggagggaatctaggggttttatctagctaggaggtcacatgactagggatccgatgtggccgaattgatctgacaagccaatagatctgacgaagccaaggtctaggggcccgaggtcttgtgagagaggtctcgagaggtcacaatgctagccacacaatatctatcaatatatgaatatattatattatatgatttaccctagatagcaatttatgccattaaccagtactcctgccgtgatgttgctttgtagtaggaaaaccatactaggttgctaattatctagataactagataactagttagttgcctagttagaactcgtatctcaaatccctgttacgtatctctctacccgcagtcctttttagatcttgttattgagtctcgtagaagtagcacatccgcgctacctgcagctggaccagctatgagactgacaaaaaacatccttaccataactcgtaagctcaagtgtttattttctgcttcaagtgcttgagaaaatagccccacggtcaagaaaaatccacttgatgtaccagtcatctcattaatctgtctgagtctagcatgtcgtgcagcgatctcggaacacggaaactgcgagcaatcgggtacaccaaggaggctattccctatatgaaagggagcagtggcgtctctgtgaaggagagtcgccacgatcgctaccataaaaatgccaatgtggcttataccagtgcaccagaaaatagtccttaggaaagccttctcttgcctcctcggccacgctgttactaatttctcggcacgatattgatttaggatccacagtgaaaagacgggaaaggcagtggaaagtccaactgtgtaagagagatagcctagtgcggccaaacttcttcaaaaagtaagcatagtcagtgagtcagagttaacagggaatcacatactcaaacttgcggaggaatgcgccatgcggtacggtctcatgcagaattatcaaaatgagcccaaccagctgagcaatgtaaagcattaggtgaagccaaaaccaaggcccattatcccaaatggactgcatcgacgcaacagcgcgaaacccgaaccatggtgatgtggttccatagcttaatgtagcatccgaagaatcaatgaactgtaatgggcagggaaagtcaatgatcggatatccttcccgtgacttccatattacgccggctaaacaaaagaaaccctgcagagagataaagatccaatcacttcgcgacatagggaaaaatagaggaaaactgataataactttaggtccagtttcatgcaatattgggaaaggccagaagcataatccgtacaatcgtcatgatatcgtcaaagcgagactaagctgtttctttataggggctgagaaatcttggcaataggaaaccggaagaatgccgagtgcgactgacgcaaagaattggcttgagcacccgaccccctctccatccctaacccgtgtcgtcattatctttcggcaatagatatggcgtttcatttcactgtaacatacagattactccgtatttatgtaataatacaccctattacatgtaatattacacgtagggagggggtgattaggaagcgtgcggatgatacgtagaactactatataattaactactccgtatagatagctagtattagttattgtaaaggtaggggtcaatatagatgattaaaagcgttcaatttagtcaattagaggtgcagacagcacctgagttttgtacctaaaaggtacatagtgcgctatagtaatgactagtttacggaggtacttctaatacattgtatccactcgttgtcttagagagagttttatcctagtcaatgcgcgctgcctcatacatcctaggctttaagggagctctccctgacagttattgcagctaccttagctacattcaggggtgctatttacgcataagggtgtgcttaataaacacacccctgtcaatacccaagccacaataaagacagtttttgtctttgtgcagattcgtgaatcctactaaagcttacagacacatgcaataccactaataaaatattgatttggagttgttttggaggtggattttagtataggactataaccactctcctatcttacatcagaataaacccaatttttgtggtctagacaaaacgtaatgctaagcaaaaagtggagagcttgcaaaagccagagagaagacatggcgccataactaaattgatccttgtatatctgatgcagttgccactgcgtgagagataaagcaagttaatcgattagtatccgatcaaaacttttcgttctaggaaagctttatttcgcacacatcaatgttcttggaatgctaacccgaatcgcaattatctgaaaccatgactagcgacttcaaggtaataatcgtgggaggatcagtggctgggctttcactagcccactgcttagaaaaaatcggtgtttctttcatggttctagagaagggtaatcaaatagctccccaactcggtgcctcaattggcattttgccaaatggtggacgtattcttgatcaactgggcatcttccatagcatcgaggatgaaatcgaacctctagaatctgctatgatgagatacccggatggtttctctttcaaaagtcaatatccccaagctttgcatactaggtaataacagtgaaagaagagtggcctataagtgttcatatatcgctaacttcgtgcggttaatagttttggttatcccgtggctttccttgagaggcaaaggtttcttcagatactttatgataaactcaagagcaaagactgcgtttttacaaacaagcgggtagtcagtattgcaagtggccaagacaaagtcacagcaaagacttcagatggcgctaagtacttagcagatatcgtgatcggtgctgacggggtccacagcatcgtcaggtcagagatttggaggcatttgaaggaaaactctcaaatatcagtattagaggcaccgaacgcaagtaggttaacctaggattaattgcaaagaaactttactaatgagggagccacttaggtattaagcatgattattcatgcatttacggaatttctttaaacgttccccagatcatcctaggaatacagttaaactgtttagatgacggagtgtcaatacacttgtttacgggtaaacaatccaaattattttggtttgttatcatcaaaacgcctcaggctagctttgctaaagtagagattgacaatacacatacagcaaggtgtatctgcgaaggactgaggacgaaaaaggtttcagataccttatgttttgaagatgtatggtcaagatgcaccatattcaagatgacgcctcttgaggaaggggtgtttaagcattggaactatggccgcttagcatgtattggtgatgctatccgcaaggtatgtggatgatgctatatgtccctatttcgtgtcatcagtgggatgacaaaagaaggccactatttgccgctaatataaatgatcgtatcgctaacattaacagatggccccaaataatgggcaaggagcaaatatggcgatagaggacgcttgcagtctcgcaaacatcctccagaaaaagatatcacatggttcgattcgagaccaagatatcaattcaatgtttcaggaattctctatggctcaacgggctcgcacggagagcgtctgcgcgcagtcggagtttctagtccgcatgcatgcgaatcaaggtattggaagaagacttcttgggcggtaccttattcctttcctgtatgacgcacctgctggtttatctggattttctataagtggcgcaacaagaatagagttcatagacttgcccactagatctcttaggggagcgtggggaaagtcatggagagggtcatgggaattcatcctacaaagcttggtctatttgcgacccaagtttaggatagtttatgccttgtatctcgttgcagctgcagcttttatcttgtattgtcttagcagtctcttcccgtagcaaggaacaactgtcgaaaatggccttaatctggaaaagctaatgcggcgatgaaggcaggcagaactcaaaaacagacaagcaatgaccctcatattgttaaatgctagttgttacataacttcatgtgattcgaggtgaaactatattaacccattttccaactaggagaaaaatgtgttatagaaaagtaagcaaatagctagtaagaatataataaaaagctagacatgaacttatatttccaacagcaagacctaggtatatagtaactaaaaggtattacgaacctaacatatactaatagtatataatagagtagcttatgtagaaatataagtaaagaaatagcaaataggtaaggaattaataaacctaataggccatagtagcaccatttagactaaacacaatatagttagctatagttatgtagtcataactaagaattcaattaagtaaacacttagtaagatagtaataagttactatagagaatatagagtctatatccttatccttgttcatagtgtctataagctcctagagctattctagaatagcaaaacgattagcaaaattgccctcaagtgtaagaatagcctagtgtaaaaaccatagcgttaagaaactataagactagtaaaaaaaagggagacttgtagtcttgcaggtattgcctctcttattacactagatatagcgctttaaagtttagtcttagctagagtagaaattaaaacctaatggaaactcaagttgatttatagtaatatagccttaataaggggttttttttaaagtccgtgtacttagtatgtaaataacacatatagctacacttttcaaaggaaattgtagttatattagtggtaaaacggtggtaaatagaagggttaaagagggtatgaactaagcttaaaaaaaccctaggaaagaaactaggtttatagggagaaaaacctaatcaggcaatagggaactgcaagtaaatgttagagataggatacttacaaaataaagggctaggaaaactttagatcctttagataattaagcagctagctagctatgggatagctatgtgtttataaagcaaggtatttagcaaagactacttatactatatatagtaaattagagtttaagacctttacacacctactcctaggtagtatctttctagtagtaactacgaatcttagccttcaatctattcattaccctataaccgaagttataacaaatccttaaatttttaataagtattaatctatacttaacacatataagtactatatttatcaagtattaattaacactataaaggttataaatataaattctacttataaaaaggaaatatatcttctttaaaataagggctaattaattaatttaatgacgcatgaaaatattattgttataaaggaaaaggggggattatttactaccccttaagttatataatcatgcgttgttagaaatattaaagcttctagtgtaaaataaaagctaagtgcaactaagtgtaattaaaagcactaggcttataacctataagatagtggaaaaagtaataataataaattcagctatctaagctctttatatacgtggtataataaggctatataacgagagcaaaagacagtctttaccctaagtgacaaggtctcgtaattagccgcgaagagggaaagcatcgcgatgaaagtgatgcctaagatgtgaggctgctacatctaacagatcagacccttcgtctcctcagaacacgcggtttgaaaagttctacctctagcaactcctcgcaccaagctgtttctacatgctcttaccgcaatctaaactgaaacccaaaattcacctcgcacatagcccctaatccgcaattgctttaacatgcaatacggtaatttaacaactgtattacttctgcgtaatactttattgtccttgaattcttcgtcaatctgccatgttcactggctgcaagtgattgtggctctgcttgtcttgatcgtctgcatctttctatattggcgaacacccactggcatcaatgctcctttcgcaggatatcgttcaccatgggagccgccgctcttggttcagatgcgttacgtcttcaacgctgcctcaatgatacgcgaaggatatgctaaggtatgttttatcccgcgtagaggtcttctacccggatagaccgagaagataacaacttcggaacagtggaaagactccttgttccagatctcacgatacgacggtgacattcttattgtgcctccaagatatttggatgacctccacaacaagtcacaagaggagttaagtgctatttatggtttgattcgggtgaggaatgccaccaaccaaaaaacgcagagcctattagcgcatggtctcacatattcgaatttgctagaattttggtggtagctatagcggcatcaccctgcttggagaaaacgatgttggcattcgtgcgcttcaggtatgtacacccttccaaaagtctgttagggaccttccttactctactacagacaaaaatcaccccaaatcttgcgaaattatgcgatgacataagggatgagtttcagtattgtctagatacagacttcccagcctgcagaggtatgccatttccaaaatcccattatgcagtctctactttttctggcactaacgatatctaacatagattggacatcagtgtccgtgcatccattgtttctaaaagcagtcgaaaggataacacatcggatttttgttggattgccattatgtcggaatccccaatgggtccaagcgaccagcaagcatgcacattacggtacgtcaattgactaataataggcaatatacgcgctcatatgctttgcagcaacaatgatacagatagctatgagatctgtcccaaagttcattcagcctttactaaatttttgccttccgtggccatggaagaacgcagcctgtgttcgtgaagcaaagaatgcccttatattagaaatgcaacgccgacgaaatctcgagaaagttaacagttttgattatatcaaatccaatgacttgctgcaagcagttatggaaatgtcttctcctagtcatgaggatagccagcttgatgttgtcgcccagataatgctcacgatgaacacaatcgctggccacagtactgccgcatccggagcacatgcactgttcgatatggttagccactctaagtatattgaattgctgcgtgaggaggctcttcaagtctttcgacatgttgaactgcgtgttacaaaacaggctttgggggatttgcgaaaattggacagcttcctcagagagttagtattgtcctaaacatcacaatctcaccacattctcacgctagcttttcctccgtactaatgatggtcgttgctaagatcccaacgacataatccgctaagcttgtgtatgtttagctaagagtctcgaaaacctggaaatgtttgtcctgtgcccgagttctaacgtctcttactacagtaggcttttttcgggtcgtattagaccctgccggtatcacacttcaagatggcacacatgttccttacaacacactgctttgtgtcgcaccacatgcgatatccaatgacccggatgtgatagaagacccaacctcgttcaacggtctgcgatactacgaacagcgctgtcgtgacgccagtcaagagaaaaagcatcaatacgctactacggataaatctcacctgcattttggctacggaacctgggcctgtccaggccgcttcttggcctctgatatgttaaaagtgattctaacgatgcttctgcttcagtatgacatccgctcccccgagagagcaaaacggcctgtggcaggtcattttcatgagtttccgcttttcaatattaacacaccactgttaatgaaacgacgcaatgattcgctagttctatgatttattgtgactttcgttagcatattacatagtgcgaaacttaatctagaaaactagagaatgaatatctttggcactgtcatgcatgcacgccttaacatcatattcatttatattattactaatggcctagatcttatttacttagtgaaactaggggaacacatcactttctttgtcctagtgtggttttaaatgttattctttgcgtacatttccatatagcagcccgtttagtaaccgtattcaccttgcctaacaatcgttttctaataacacgctaagggcaacaagtgacaagtgtttagtaattagtaagcagtttaggttagggggagcaaggtagtgtaagcgcagggcgtgcggtttattataatagaaaagaatatagtattagggttaacactagaaaaatccccctagcttattaagtaaggaaatagattagataattatagtagtaatatttatagaatcgctctagctagcttaagtagtaattaaccatcatcattacctaatcattttggtactattacaggcctttccgtacagcca

SEQ ID NO: 24 - DNA, 11202 bp, Neotyphodium lolii
atttatgtcttttgcagcgctgtcgtataattaagagcaattatggctcgttgcagcaaacaatcgcccaattgatacaatcaaaattccacaagcgaaagttgtgacaactcacgtcctatcactcctgtcgtttcctttcaacatacggtaactgtcttctccaagcggcgcaaaagaacaacggcattccgattggggaggatcttatccccatgaacaatgttaggagggcgccgcgacggaaactcactatctagcttaaaatcatactcgagcagaatccgactcatgattgctttaatcatatactatgagcacaaactcaagggttagtcaagaaggtcgaatgtgcgtcacatgagcttgaagggagattgatatggaaaaaaaaaagatgaaaatctacatcacatacggaagctatgaagcgtcccggacaggcatatctcccaaatccgaagtgcagattcaaggcgctgctgttagaaaatgatgttgtggcagtgccagattgtttcaaccatcgaaaaggttgaaacaagtgagcgtcggataatttcgtactgtccgtattgatacacttcgctggaatcgcaatctgctgtcccggctgtagctggtagccatcggacaatgtaattcgttctcggacaatgcgtctaaaagtgacttcaatcgccagttagttccaagtttgacaaaagactggcttagaatagtccttacactcgcccactggattgattcgttgtgactctctcatcaggctatccaattgtttcatatcaagcatagtctgttgcgtgatttccgtccaaccatgcttgagcacgttttgctgttctcttcggaggctctccttcaagctgtcgtccgagatcatgtcaaaaagtatgtgagttaacgccatagctgttgtatgaatgacagccatgctcaatattcctagcgtacgatgcgcaagtgcttctggccgtgcttcatcctcggtgactgctaactctgtacaccactgcaaaaagtcgtcgtgcttttcctcagagttaagtttccttctctggacttctgaaccaaggaggttttgaatgtattgtaattgcccgtgagcttctttcgctgtggaaatccaccgtgcgaaataatgtgtgaacaaattgccaaaactacgggaggtggatatcgtatcaaacgtgttgcgactaaaagaagcagtagtctcgagaaaaatttcgtttcgacaaatcaggtctccaacgaatagcctcgctgtagcacgggtagctagattgagaaccaattcgtatggattgacagaaatccaagcattggaacctccactaatattagacaacactagacaagtggaatgtttgaaagaatgaaaacacataccgtcgttgccggcaggtaagacttgcccaaaggcatgattgagctcatccaatagcactggaagagtttgtgctggattcagttagcatagtgcaaacgtattctgttagattacacttacgaagctttggagtcaaacgctgctgtatggcacgtgtgtgaagttcgctccctaataatatcctcgtaactttaccagcattgtcatcaaagctggctgttaaagggtctaatttgtcgctcgtaaccgcccgtaattccagcgcgtattgtaaaggaatgacaatcagatccatgtccatcttaagtagcctaaacggttgatttttgtgctgtaacctaggcggttagtactatcatcacggatgctcacagacttgggaccgaacaccttctcatagccatgtttgactaggcgggcggcatggtcattataactcagccgatcgaggaattgggggagtaatcgaggccaacgtcgaatggttggtatacattctttccgtttggtaccgtaaacaacgtaccacagtagtaaacagatacccactgggacagcgtgcaacattaacattctcaagagtagctgatttgacttgaatggaatataaaatgatttatgaattaattttgaatgggcttggcatctacagaataaaagattataagacaaataagacaaagcttaatgttaaaactttatgttaagtacttgaaattgtcctaagccatcgaatctaatgcaacgcctgtcctcttttctcttgacactatgtaaggagcctgcagcaataactaaggatgtgatccaagttagcgcagttatttaaaattcgtcatttttagacccactagcgccttcctatatttagacagtattttacggtatataacctagataatttcacccttgcttataatacaatacaatccctgaattgttttacaaatctatataatagaagtaattgagctaattaaattatagctaggaaataaaggagacagggggtggtatattttagtactagaacctgcatagaaatagatattctcttttgtgacgctatataccttgcatatttcccttgtagctctctaataataggattacttatagctaatcacagccgttagggaggaatcaataactagggcatgtagacttgtaaaataattcagcgggtagagtgtgtacttaaattacagtggtgttacaggggctatttagatagccaaaagagggaagccctatttctgacactggcgtagtaaaaaaaaaaagtgcgctaatgtattactttattcttacggattagtatctgatcctattgcaggcatttacttggcactagttgaaaagatatattataaacaggggggagtggttttatgcaatgtgaacaaagtttcacaaatttctactccgtataaacataatttattgggggtcttgacatgtccgtcttagccgacaaccccaccatgccacgaatcccgcggagaccccaatcaatccatacacggcacttacacagatcattccatccgcccaagtaccgtcctatactccgtacaccctaaaagccttagagcacgaacatatgccgttgtctccctagtcacacacaagatgcctaccccccttcccgattccccttctcacatgtgtaacgtatgtaacgcaaggttaggtcgcggtggcacaaaaaagtaacgccgcagccgaaagccatcctgtcgccagcggaggtgtcgcggttcgtatctgcttagctgtgtttcattgttaggcgtgtgcataatgcgcggggtgcgtttaaatgtctagctgaagtcatatctgttgccgtgcatcacatcacttttacttcgggcaccatttcatgcaccctaatagccacgacacacagaatccatcaccaattaactcaggcagttcgcacctacactaagccattcgaacaatatacattacttcaaagactcaccttaggccgtcttttcacgcagccaagaagtttgaacagctcgttcctacatccctgcgaatcggacgtttttcacggatagaccctctaggcccttaatgaagacttctaaatgtcaggagctatctaagtcaagtacgttgacaatacatttctttggaagtgttgtcttccgttttcttatccctcttatccctttagcctaggttttctaaagttaaagtcgtcaagctaggttcgatatgaagatgttaacagagcattttgactttcctaaacttaacttcgccaccattgtaatttcaggcgccaccattattggtataatattccttcgatatcttaattaccctacaaaggtttgaatctgtttcccttgctatgcacttttcttggatgctcaccaaaatttttcaaggtgaatgtacctgttgttggcattggagttcgatatacaaaatggctagcggctattataaacgtgcgtcatgctcgacaatctatccgcgagggctatgcaaaggtttgtgttaaaaacgaataaaagcgcttcgtaaacaaagagaactaatactagtttctagtatggcgatttcgcgtttcagatacctactatgactcgaatggaggtattcatttgtgatagacagatgacaagggagtatcagaatgttgacgactatcatttgtcgttccgagctgtcatgaccgaggtaagtaactagaccatgttaactgtaggaaaagaagaaaaagctaaaccgccgtacaggagtttcaattcaaatggctacttccaggacaggcacacgaagcccggattatccctaactcagtgattgctaaggccttgagctggcagagaacaagggcgaataaacccagcgatccattcttcgaatctttctccgccgaattcatgcaggggtttcaggaagagatgcgacgactaatccaatatcaaaattcgtcagttatgtcaaaccgctccggtgctgtcctggatccagcgcatggttggcatgctgtgccttgttttcccttggctctgaaggtaattgggcgccttactacatacgtcttgttcggcaaacctttgtgccaagatgcgacattcctaaacatgtgctgtcaatttggcgatgtgattcccagggatgcgatcatactacgttcatggccagcattggcaaggccgtaagcaagtgcctagacataaacccgtcagggtttaaactcgcattaacattcatatagtcttattgtaaagatcttgagtgctccaagggttatgggaaagttgcgaaacattttgattgttgagataaagagcaggagagaatcccacgaaacgaacccaatgagtgtatggctgtctcgcacaccccctctagcattacacattaacgtatatctaggatatcttggatttcacaatggcctgggttgaccgtcatcctaacgctagctttgacgatcagcacattgccgagatgatgattaacactattttcgcagctcttcatacgtcgagtcaggtatatttttttctgtatgaaaagtccagagcttaaagctaactggctcatagctggtggtgcataccatctttgagcttgcctcacgtcctgaatatagcgatgcgcttctggaagagatagatgcatgctttgaaaagcatggaaagggcactaaagcagctctagactcaatgttcaaggtggatagtttcatcaaagaaacgcagaggtttaaccctcttgacgcatgtataaattccctgtctccgattccatcattgcgatttgactaacgccaccgtcagccgctcttgcaagactggctctcaaagactttactttttccaatggcctaaacatcccaaagggcagtgtgattttcacgccgaattcgcctatctttgaggacgagagatattacaaggatccgaaagtttttgatggatttcggtttgctaggatgcgtaatgacccaaaattaggtctattctgcgacctaacagcaacgaatgaacaaagcatgcattttgggactggacgtcacgcctgtcctggtagatttatggtttctgatgaggtcaagttagctgtgattcatatcttaagtaatttcgatttttgtattgagaattttggaccacggccagcaaatcagccatttggtaaatttcttctacctgatatgagtgcaaaaatctggctaagggagaaaagagctagggagaagaatctgtgaaagccgttaagataatgccaattgctacacgatacatatatgttcatgttagcgagttttgaagagaagctttgaggcctctaagaaattttaactacctatgataatgaagcagctttatttctaacatgatttttctagcctgtgaaaagtgatttttgcagcttagacaataggatacatgttattagcctacctgagggggctatggtaagtgactctaagatctcgcaatatcaatgaaactataggcaatatctagctaattaggcctatatctatgcttacaaatgcagtattacctctaagtctatagataacaagcatacagctagtttcgtttcattacgtaacggtttgtctctaagtagcgacagctaagtgagacagatatatcaggcacaatacaatacaccccctgagttcttttacaaatctacatgccccagttattcgttcctctctaatagctatgattagctatatgtaatactattattagtgagctataagggcaaaatacaaggtatataacgccataaaagagtatattttttttttctctatgtaagtgcctagtacaagaatgtactatcccctattaccttcatttcctatctgttatctaattagcttaattacccctgttatgtggatttgtaaaataattcagggggtgtattgtacatcattccaatccgtcttaaatcatagatatatgctccttgggctttcgtgccacacccccataagtacaaatgcactgttcacatgtttcacagcctttgattgcctagaagagacgaataggtataatagtgcacatattgccatccacttaatgctaatatccctttcgctcgctctctttatcttttgtggacgcaggtcctatttttcacatataagcacttccgaacctgcgtaaaatcttactcacgcaaggaaatacaattcaaattatatcgtgcttgattgatctctttctaggcttccttttgttcaagagactaactaacaattgttgtcttcggctctcacattacaccatgattgcgaaaaatattgaactcaatggcttggatccggcaaccagggcattggacattctatactggaaaaatcactgcatcaaacagctagaatctctcctatgcgccacagattcatactgcactgcagacaaggccgctcaactacgcattttgtcagagttggtgctccccaatcttggccctcggccgtccaatgccactgggccatcctatcttacacgaagtggttccccaataatgttaagtctaaatacaacatcatcaaaaaactgcgtcagatattgctgggagattctaggggcgactggcgcaagtaatgatgatcctttggcagtccaagttgctaaggatgtagtggcttctctgtctgctacttttcgcctttcaacaaaatggagcgaaactctactgtccaattttgcagtaacaccagaccaagctcgacaagttattaacatgctacccgagtggattcaaggcttcgtacctgagggaatggagtgcgattttccaaagagaatcccgttcgccatgacatcattcgacctaaatggctccaatgtagctatgaagctctacgttaatccaagggtaaaggagattttaactggtactccctcatcagacttggtctgggagttcctccgaaatttaacaccagaaatgaaaccacgagcggtcgacttgcttgagaggtaagaatggctttgaactttcgcccaccttgtcagccccatacgctaagcgctaactccccacacattaacaggtttattaccgataattcaggcccgtctgctattgagcttgtaggtattgactgcgttgacgacgctcacctatcaaatgcaagggtcaagctttacgttcataccatgagcagctcatttaacaccgtaaagaattatgttactcttgggggtgcaatctgggatgaacaaacccaaaagggcttaggaatactacaaagtatttggcacctattgcttcaggagccagagggtatttctgacaatggattcgacaagcctgtgaacgactcttccatgttatgccaaaagctatattttagtttcgagctacgcccaggtacagacttccctcaggtgaagacctatgtgccaacttggaactatcttcgaaccgacggggaaactatccagaactatgaggcgatcttccgagcttgtgaccatccttggggtgaagataggacgtacggcaaaatttttcaagatgcattgtaagttatcccttcagattagcgctaaaaggagtttgagatactcctcaatgcaagctattaggttgtgaaattgccactactaattggagctttttatagcggacctgcaaccgagagtcggaaaaaacccattcactgcgacgcatcttttctgtttaccgaagaaactggtgtctaccagacgctgtatttcagtcctccgattgagggggaaacagaagtccagtcaaatctcgttgcttgaggttgaattaactccgcaatgctacgtctaaaagaagtgtctttggtgaacagatgatagggttcccttgatctttcatatatttgtgtacagctgtggaaatttagggtctagctctagataaagccattgcttcaatcgtcatttgacgtattctgagtcttgagctattccatattttttttctaattaatcttgactttattaagtgttgtaggccgttgcaataatatttgctttgatcttacaagtgtagcagctacccttgcactcttcgattcttgaacgagcgttgctattcggagctgtgttcaagaactaggttgtgcgcataggtttaattttgcaatatcacgaggagaggcccgttagccaactgcttaaatacaggtcttgctagaaaatggttgccttaatacagctgctatgctacctcctatctccttaagcgtgttctaccttgtgggtctaggctttggtaaagggtagttattacaggcaagagatgtcacatcaagatagtttttgtctagcatagcgcgtgagttacatttctccgaaatcattttgtaaagtgcatttgttcttttcgtccccagacggccaccaagttctctagaccctgacctgcctcctggagcgttttgagacacgtgtttttaacactaggtgactctaacgcttggagtgcccgtttaatgtcgccttgattttggctctgaaacgccttccttatctgcggctcagtatgtttgtcattaagagcaaccacaacaggaaaggacaattctctgttgcgtaggtcttctgcaacagtgcctttgttaaaagcgtactcttctgagtatatgttcttgcaatcattttgcaattgtgcgtaccagctaccattccattgaattagttaagcgaccttgccgcggaattatgaaacgagcgaaaaaaaaaaaaatttacatacccaaatcggcctaacagatcgtctgattgatgacctccttggttcaaaagcctcccaaggagcacaaaaagtgtgcctgtcttcagcagagccatatttttgtacgtcaacaggctttcttcaccataggattcgaaagatcgcaaaccgtctcttcgccaaaccaaagacatgtcttgtccctcgagtatcagttctagtgctctcagaagttcaattccaaggacaggttgttctttcattgctctattaatgacctttgtgagaacgaagtatgctcgattggcagtttcgcatgatccgtatagcaagtgagccgtagtgtgatttttccgcttagggctgtggtcgcaaatatcgtcaaccataattagacaaagatggaccgcatccatgatatccaggatcaaagaatgtttcttgggatatttatgtttcaatgttggatgaagaaaatccacgaggggagagaaatgattatgcccatagagactaatcacataagagtatggacagtcaggaatcttgccattgctgccgccaataccttcgtgagaggtacgttcatagccatagccataatgatcaatggcttgagaaacgacagattggtaattgattgtgtactttactagatacccgagcgtaaatgcaaacaagagggccgcaatttcgatggccgcagggcgagccacgagccatgctccagatgtcatcttggaatagtgatgtacgtcggctaaaggcagattgccttgagaaagaattcagttaagcaaagttttatcaattctcgcaatatatgcagagcaactcaagcaaatgttaggaatatcgttagactataattatagaggcagagcttctagaatagcgcaatcatagtctattatgtatacgcccaggcgcggttaaatacatacatataattcaacgaccttgtcaggcaatcaagatgtgctactcttaattacataatgaacaagatgctagaggtattaaaggccaaaatgtgtccttctggatagcagaccggactaaaccttcgcaaaccattcctataatactagctgattttatcactatggacggattcagcaatatggagcaagcgccgctcgcttatcaggaagttcaatggctagctgaaacttttgtcactttcatggggcttggctggcttatcaattacgtcttgatgatctggcactctaggaggggtgaaccgagcagcatggctctcatacccctctgcaacaacatcgcctgggagctcgtatacacgattatctatccgtctcctaacaaagtggaacttgcggctttcatagcaggtgtcactttgaacttccttatcatgacctctgcagcccgttcggcaagatccgagtggagtcactcacccacaatggctaagcatgcaggtttgattatagtcgcaggaatattgatgtgcttcaccggacatgtagcattggcgatggaaataggacctgcgcttgcttactcatggggagctgtcatatgccaactagctctaagcattggaggcgtgtgtcaattgttgcagcagcatagtactggtgggacatcatggaaactttggtaagtgaataaatcaattacgtttctaatctatattgaatgtcatatcaggggtggctgacatgaaagttttcaggtcaagtcgatttctaggctcttgttgtgcggttggctttgcctttcttcgctggagatactggcccgaggcgtacggatggctggccagtccccttatcctctggagtcttgccacgtttcttgtggccgatttgacgtacggggtttgtctccttctttaggcagaagagaggacagtcgaactccactaagcttcagactgcgcaacgaaaacgagtaacggcttgagactagttctatcttatcgatcgctatcttacatggttaatgtaaccttctatctttgtctaagggcttacactcaaatgaaatcatacatgcaacttaaactatcatacataggagagtgccaatttaagcaatttaagcac

SEQ ID NO: 25 - DNA, 6583 bp, Neotyphodium lolii
aagctttttaccctaaattatagtataaaaaagcaaatctctcttagtaagctactttataatattaatatatatatactattactcttaattatctagtataataataagtaaataatctagattactaaatatatagaaaaaaggctttagactagccttaagtaccttaatataaattaaatatagatttaagaataatatataataggtaatattaagattaattaaataaaaaccttagagatacctatagatagctttataaagctacttttaggttaaacatataaggacttttactataaaattagattataagaaattcctatttaattataatattagggattatttataatctcttcctatacctttaatctttagctttaataaatatcttagctagggggggtatattaggaaaagctccctaaagtaataaacatataaatatagcctatataataactaggttaaaacccttaataaaataatagtaaagataatatattagaagtatctttatagactagttattactatagcgcactatataccttttaggtataaaagttaggtggtgtttacacctctagttaaccgaaatcgaaccttttctattatctacaccgacccctaccttgacagttaggactcttgttcctgcaggtcagctagcttgagcgcaaggcaccgttgcaccatgcatgtcatgtgcagcacggcgcatagatgccgatgatgccgccagtgcctatcttgaaccccagatgcagaccttgatatggcctcttgcgagtaaacgcattgcgactatacggccttgaagggcattgagttggaggctcttccctataatatgtgcttgtagaattggtatcgctgcttcattttaagcattggaaacattcaggagacgtactacgcttgtgtatgcaacgcttttctttttgaacaatgaaacctactactcgctgtccattcgactatctggtgagccagtgtggaaagcatcatttcaaaacctttgtccagttactgtcacctcttctccaagacgaagatcccgacagatatgctttaattctggacattatggacgctgtccacttctccgccatcttgatcgatgacattgccaaccaaagtgctctacgcaggaaccagccggccgcccatgttgtttttggggagacggaaacggccactagagcctacctcgttctgctgcgggtcgtcaacaggacaatgcgagagaacccagtcctggccggtgagctactaaacagcctggaagaaattcaccagggtcaggacgagtctcttgtgtggcgccgcgacgggctggagacttttcccgtcgccgatgacgagagactggcagcctacgtgcgcatgtcccgcctcaaaacgggctccctcttcgtgctcctcggacggcttctagccaacggtggtaccgagtttgatgacctgctggtacgctttgggtacgtttctgcctcttgtcccaacaaagagggccgccgactaactggttctctcttgttcttgaagcctgtacgcgcagctgcaacatgattgcaagaacatttactctcccgaatacgctctcaacaagggatccgtcgctgaagatttgcgaaatggcgaattgtcctatcccgttgtggtcgccttgattgagaacaaggcggaagggatcgtgggagaggccttgcgcacgcgcagcgacggggataccgaacaggcactccgcgttttggagagcccggcagtcaaggacgcgtgcctgcacgcactcgaggctgcgagtgtcggcttggaagaccttgttgaggcatggggacgacgagaaaaaatgagatccgacaccctcgacggcgacgacttaacaaggccaagcaccatcacacaacatgaacaagatgaccatgttgatagagctgccatcgatgccaagagtgatgcgagtggcagtagcaataagtctctcacgcccccagagacagcccctacgacggacaccctgtccgagacagctgtgggggatatctcgtcagtcgacgtggattactggactcggagatgcgttcccataatcggtagcctcttgaaatcatgccgagtctactcggaagcggaacgggaaacacagctgcgcttccttcaggaacatgtgctacctaatctaggtcctcgtccatcctcccccggctcgcagatccagtccatggctacattcagcggctttcctctccaacccagcatcaacctgagcggctccggccaggccaaagtccgctacacgtttgaaccgctcgacagcctgagtggcaccgaggttgacccttttgcactggcgccggctcagcgagtgctcgaaaagctctccacccttctcggcgtctggcctggatggatcgacgctttgatcgctgcgtaccacccaaccagagaggaagtcgagcaattacacccgaatctgcacgagtacctcagaggcgtcctcgtgagaacgacaggacgccaagatgtgcaggttcctcccatgccgcgaatgtgggtgtgcttcgttgcgcttgatctggagggcgcctcacaggcactcaaggtttattttgatcccaagatcaaagaagccgtgactggtattccttcctgcaaatacacttgtcagattctacggacggttgataggtttggcaacgccaaggccgtcgacatgcttgagcagtgagtcacgtctgggagcactatccaggtccgaggtactaacaagattttgtcaggttcttggcagaggagcacagcataggcgctgtcgaactgattgccattgattgcgtcccagaagaaatgcagccatcggcgcggatcaaggtctacgttcacaccatgagcaactcgtttcagacagtacgcaagtacatgacaatgggtggccgctgcatggatcctgcgaccctcgagggtctggaaaacttgcacgacgtgtggtactcccttctcggggaaagtcaaggtattgtcaatgaagagtacagcaagcccttgactggctttagctcgatgcagcatcacttgtactttagctacgagatgacgcctggcaatgctgatcccggcgtcaaagtctacatacctgtgcaaagctacgcgccagacgacaagaccatcgcgcagaactacgaggcaaattttcggcaactcaactggccgtggggcgaacccggcgtttacgaagcggtgatagagagtgctctgtacgtaatgacaggccctttgaccatattacttactgacaacttggaatttagtggaccagtaaagcacagccgcgcaacgttcctccatggaggatcttctttcatcttttccaaaggccgaggagtttatcagtccatatatctagaccctccactggaggaaggagggaacattgctgtattcgagcaccacgacgatcaggatactatagttgaccttggcaatatgtagtcttgtcatcaattgatcagctgtatgagctcttgtgttttttcctttagctagtttggcctgaatgtttgaaaaacatgtctgtatgaactagtgatggaagaagggttgaaagtgagcatgtaccgcaaaacattattccttcaccttgctcgagatagctcaccgtaaacgtacctggtgaggtattccagaattgagccggttcattttcgcggacatgaacccatcatcagcttttgccgatcctaaagtctagacctgaatagtgacgcagctggtatgattggtgcaggacaattactgcgcccacggcggacgcgatgccggggggccgcccggagaccccggcatgcagaacgatcagctcttgagctcctacgtcgcgcatgtctcattcaagcatgcactatatattgagacctactgtatgcagcctcgaatgtaaccgtagtattcaaacaagaaacatgcatatatttgcatgatgctttccgtggcgctgcgcatatgatatacatggtttacatatgagctgacttgaagcacggcatagccggaggattcttctgcatggagcactgtatccggctgaaaattacattgtacgaggtctcaatctgcggccagctagccgagcaccggagaaccggcgcatctctgcttgaactcgggcaagggactcacttctacaaaagtcagagatgcataccatcaactgaaatcaagttaggatttatagcctttatatttcagtggcatcactcaattacttgtttgaactacgccgtcagaggttcacctactaccagaaacgacagcaccatggcatttgcaagtcttttgcaccatatctggaaccatgcagtggattgcgctgagcagctgacttggtggcagaccattgtgagcttcatcattttctgcatcatgtgctcttggctacctgggaatggggaaatgcgcgctccgtttgttggttatcgctggccattcgagcctactttctgggtccgaatgcgcttcatctttcagagtttaggcatgatgaccgaaggatactcaaaggtgagctcccgtccgggtggagaaagacagctagacgaatgactgacgccaaacgcttgacagttcaaggattccatgttcaagatcacgaccaacgatgccgactggcttgtcctctcccaacgctacttggatgacttgcagtctctgccagccgagagattgagccatacagacgctctagtgacggtgagggcgcatactagtcgctagtccctacgacagtggtgtgctaatcgagttgtgtctcatttagatgtgggggagcagccacagcccttttgctctgctcaacaagagtgatcttagctctcgagctcttcgtgtaaggaccaatccctccttgttatgcagaacggatctgacttgaaaaggacgtggttgcgccgaattatgccaaggaccttgatagcctcgtagacgaactccgctattcgcttgagcacgatatagacatacaggatggtatgtatgcgcctattttccaactaattttgaggtcgtcatgttggctgactgggtcgatgcgcttagactggaaaccgattgatgcccttgaactttcttcgaagttggtgttgcggatatcgcagcgaatcttgatcggctggcccatgagtcgcgatcaagagctccttgaatgcgcacaaggctacgcagacgctggtaagaggacgagctgttacgtatgacccttttcttcggtaaaaactaacgggggtttcagctaccgtcgtccagtttgccctgaaactacttcctcgccagattcggccgcttgtctatcctctgctcccacaagcatgggctactaaatcgtggatcaggcgctgtgacaagatactggcaaaggaaatgcaacgtcgacaagttttggagaagtcggatcccgtgtacgagaaaccaaaggacttgctgcagggcatggtggacctggagccgtcccggcctgttgacaaacttggacatgattttctcgtccaagccttgatttccagaatggctccagttgttaccatggcccaaacccttgttgatcttgccctccatcctgaggatatcgaggagctgcgtgatgaggttctgcaagtcataggaccagacggggcgggattaggaaacctacgacaatcatttaccaaacttgacaagatggacagcgtcttgagggaatctgccaggttcacccctctatctatgagtaagtgccatttctgtcctccagaatagcttgctggcatgactaatctgtggtatagtgacaatgcaccgccgggttcaggacgccaagggcatcacgctccatgacggtgtgcatcttccacgaggcacgcatgtggcattcccagcgtaccacattggcagagatcccaagttggtgtcaggtgcagatatctatgacgggctgcgctggtacaggaaggacctcggcgaggcccaagaaaacgaagctcccaagcatcgatttgtcacccccgacagcaactacttgacctttgggtccggtaaatacgtctgccccggccgatttatagcggaacacatgttgaagctgatgatgaccgccgtgctcctgcgctacgagttcaagtggcctccgggagtccctgtgcccgaacaacagtatcggcatgtctttgcttatccaagcaaaaccacactgttgattaaacgacgcaaagatggcgatcagattctttaaagtatcattatctgaaaagaagaaaagaggatgtcttcctcttcccgttaaagactgctgagtgcaagtttgtgaaaggagaggtgttacgaacagaatgtacatgcccactagaacgagttagagtatggcagctaccttgactaatatgttaactttaataatatataattgattattaattgtttttaaatatttagtatttaataaaaaatagaatattgtattttatataaattataattaaacaatatattatgtttaataatataattaaatataaaatacttttattcaagattataaaac

SEQ ID NO: 26 - DNA, 20 bp, Neotyphodium lolii; Epichloe festucae; Epichloe typhina;
  misc_feature (3)..(3): y is t or c
  misc_feature (4)..(4): m is a or c
  misc_feature (6)..(6): n is a, c, g, or t
  misc_feature (12)..(12): r is g or a
caymgnggtcarggtatgga

SEQ ID NO: 27 - DNA, 23 bp, Neotyphodium lolii; Epichloe festucae; Epichloe typhina;
  misc_feature (6)..(6): r is g or a
  misc_feature (15)..(15): n is a, c, g, or t
  misc_feature (17)..(17): k is g or t
  misc_feature (21)..(21): y is t or c
ttcatrtagtcgtcncktatytg

SEQ ID NO: 28 - DNA, 23 bp, Neotyphodium lolii; Epichloe festucae; Epichloe typhina;
  misc_feature (9)..(9): y is t or c
  misc_feature (12)..(12): n is a, c, g, or t
  misc_feature (15)..(15): s is g or c
  misc_feature (17)..(17): r is g or a
  misc_feature (21)..(21): y is t or c
aactttccytcngtsargtcytc

SEQ ID NO: 29 - DNA, 24 bp, Neotyphodium lolii; Epichloe festucae; Epichloe typhina;
gcttggatccgatattgaaggagc

SEQ ID NO: 30 - DNA, 24 bp, Neotyphodium lolii; Epichloe festucae; Epichloe typhina;
ttggatccggttcccggtcggcat

SEQ ID NO: 31 - DNA, 18 bp, Neotyphodium lolii; Epichloe festucae; Epichloe typhina;
tggatcattcgcagatac

SEQ ID NO: 32 - DNA, 18 bp, Neotyphodium lolii; Epichloe festucae; Epichloe typhina;
gtgtgagattaagacgtc

SEQ ID NO: 33 - DNA, 18 bp, Neotyphodium lolii; Epichloe festucae; Epichloe typhina;
accgacgccattaatgag

SEQ ID NO: 34 - DNA, 18 bp, Neotyphodium lolii; Epichloe festucae; Epichloe typhina;
actgggcatcttccatag

SEQ ID NO: 35 - DNA, 18 bp, Neotyphodium lolii; Epichloe festucae; Epichloe typhina;
attagaggcaccgaacgc

SEQ ID NO: 36 - DNA, 18 bp, Neotyphodium lolii; Epichloe festucae; Epichloe typhina;
atcaagctggctatcctc

SEQ ID NO: 37 - DNA, 18 bp, Neotyphodium lolii; Epichloe festucae; Epichloe typhina;
aaataatgggcaaggagc

SEQ ID NO: 38 - DNA, 19 bp, Neotyphodium lolii; Epichloe festucae; Epichloe typhina;
tgggaattttggaaatggc

SEQ ID NO: 39 - DNA, 18 bp, Neotyphodium lolii; Epichloe festucae; Epichloe typhina;
gctccttgcccattattt

SEQ ID NO: 40 - DNA, 18 bp, Neotyphodium lolii; Epichloe festucae; Epichloe typhina;
gtcttgatcgtctgcatc

SEQ ID NO: 41 - DNA, 18 bp, Neotyphodium lolii; Epichloe festucae; Epichloe typhina;
tgtccgtgcatccattgt

SEQ ID NO: 42 - DNA, 18 bp, Neotyphodium lolii; Epichloe festucae; Epichloe typhina;
catagagctagctagagt

SEQ ID NO: 43 - DNA, 18 bp, Neotyphodium lolii; Epichloe festucae; Epichloe typhina;
gttcggtgcctctaatac

SEQ ID NO: 44 - DNA, 18 bp, Neotyphodium lolii; Epichloe festucae; Epichloe typhina;
gaggatagccagcttgat

SEQ ID NO: 45 - DNA, 24 bp, Neotyphodium lolii; Epichloe festucae; Epichloe typhina;
gattggtaccttgaagtcgctagt

SEQ ID NO: 46 - DNA, 25 bp, Neotyphodium lolii; Epichloe festucae; Epichloe typhina;
gtagggtacctctagtactgcctct

SEQ ID NO: 47 - DNA, 18 bp, Neotyphodium lolii; Epichloe festucae; Epichloe typhina;
tagcgaatcattgcgtcg

SEQ ID NO: 48 - DNA, 20 bp, Neotyphodium lolii; Epichloe festucae; Epichloe typhina;
atggctgccaatgactttcc

SEQ ID NO: 49 - DNA, 20 bp, Neotyphodium lolii; Epichloe festucae; Epichloe typhina;
aggccattttcgacagttgt

SEQ ID NO: 50 - DNA, 20 bp, Neotyphodium lolii; Epichloe festucae; Epichloe typhina;
ccagcaagcatgcacattac

SEQ ID NO: 51 - DNA, 20 bp, Neotyphodium lolii; Epichloe festucae; Epichloe typhina;
tgcgtgagagataaagcaag

SEQ ID NO: 52 - DNA, 2544 bp, Neotyphodium lolii
atgaaacctactactcgctgtccattcgactatctggtgagccagtgtggaaagcatcatttcaaaacctttgtccagttactgtcacctcttctccaagacgaagatcccgacagatatgctttaattctggacattatggacgctgtccacttctccgccatcttgatcgatgacattgccaaccaaagtgctctacgcaggaaccagccggccgcccatgttgtttttggggagacggaaacggccactagagcctacctcgttctgctgcgggtcgtcaacaggacaatgcgagagaacccagtcctggccggtgagctactaaacagcctggaagaaattcaccagggtcaggacgagtctcttgtgtggcgccgcgacgggctggagacttttcccgtcgccgatgacgagagactggcagcctacgtgcgcatgtcccgcctcaaaacgggctccctcttcgtgctcctcggacggcttctagccaacggtggtaccgagtttgatgacctgctggtacgctttgggtacgtttctgcctcttgtcccaacaaagagggccgccgactaactggttctctcttgttcttgaagcctgtacgcgcagctgcaacatgattgcaagaacatttactctcccgaatacgctctcaacaagggatccgtcgctgaagatttgcgaaatggcgaattgtcctatcccgttgtggtcgccttgattgagaacaaggcggaagggatcgtgggagaggccttgcgcacgcgcagcgacggggataccgaacaggcactccgcgttttggagagcccggcagtcaaggacgcgtgcctgcacgcactcgaggctgcgagtgtcggcttggaagaccttgttgaggcatggggacgacgagaaaaaatgagatccgacaccctcgacggcgacgacttaacaaggccaagcaccatcacacaacatgaacaagatgaccatgttgatagagctgccatcgatgccaagagtgatgcgagtggcagtagcaataagtctctcacgcccccagagacagcccctacgacggacaccctgtccgagacagctgtgggggatatctcgtcagtcgacgtggattactggactcggagatgcgttcccataatcggtagcctcttgaaatcatgccgagtctactcggaagcggaacgggaaacacagctgcgcttccttcaggaacatgtgctacctaatctaggtcctcgtccatcctcccccggctcgcagatccagtccatggctacattcagcggctttcctctccaacccagcatcaacctgagcggctccggccaggccaaagtccgctacacgtttgaaccgctcgacagcctgagtggcaccgaggttgacccttttgcactggcgccggctcagcgagtgctcgaaaagctctccacccttctcggcgtctggcctggatggatcgacgctttgatcgctgcgtaccacccaaccagagaggaagtcgagcaattacacccgaatctgcacgagtacctcagaggcgtcctcgtgagaacgacaggacgccaagatgtgcaggttcctcccatgccgcgaatgtgggtgtgcttcgttgcgcttgatctggagggcgcctcacaggcactcaaggtttattttgatcccaagatcaaagaagccgtgactggtattccttcctgcaaatacacttgtcagattctacggacggttgataggtttggcaacgccaaggccgtcgacatgcttgagcagtgagtcacgtctgggagcactatccaggtccgaggtactaacaagattttgtcaggttcttggcagaggagcacagcataggcgctgtcgaactgattgccattgattgcgtcccagaagaaatgcagccatcggcgcggatcaaggtctacgttcacaccatgagcaactcgtttcagacagtacgcaagtacatgacaatgggtggccgctgcatggatcctgcgaccctcgagggtctggaaaacttgcacgacgtgtggtactcccttctcggggaaagtcaaggtattgtcaatgaagagtacagcaagcccttgactggctttagctcgatgcagcatcacttgtactttagctacgagatgacgcctggcaatgctgatcccggcgtcaaagtctacatacctgtgcaaagctacgcgccagacgacaagaccatcgcgcagaactacgaggcaaattttcggcaactcaactggccgtggggcgaacccggcgtttacgaagcggtgatagagagtgctctgtacgtaatgacaggccctttgaccatattacttactgacaacttggaatttagtggaccagtaaagcacagccgcgcaacgttcctccatggaggatcttctttcatcttttccaaaggccgaggagtttatcagtccatatatctagaccctccactggaggaaggagggaacattgctgtattcgagcaccacgacgatcaggatactatagttgaccttggcaatatgtag

SEQ ID NO: 53 - protein, 788 aa, Neotyphodium lolii
  misc_feature (185)..(185): Xaa can be any naturally occurring amino acid
MKPTTRCPFDYLVSQCGKHHFKTFVQLLSPLLQDEDPDRYALILDIMDAVHFSAILIDDIANQSALRRNQPAAHVVFGETETATRAYLVLLRVVNRTMRENPVLAGELLNSLEEIHQGQDESLVWRRDGLETFPVADDERLAAYVRMSRLKTGSLFVLLGRLLANGGTEFDDLLVRFGLYAQLQXDCKNIYSPEYALNKGSVAEDLRNGELSYPVVVALIENKAEGIVGEALRTRSDGDTEQALRVLESPAVKDACLHALEAASVGLEDLVEAWGRREKMRSDTLDGDDLTRPSTITQHEQDDHVDRAAIDAKSDASGSSNKSLTPPETAPTTDTLSETAVGDISSVDVDYWTRRCVPIIGSLLKSCRVYSEAERETQLRFLQEHVLPNLGPRPSSPGSQIQSMATFSGFPLQPSINLSGSGQAKVRYTFEPLDSLSGTEVDPFALAPAQRVLEKLSTLLGVWPGWIDALIAAYHPTREEVEQLHPNLHEYLRGVLVRTTGRQDVQVPPMPRMWVCFVALDLEGASQALKVYFDPKIKEAVTGIPSCKYTCQILRTVDRFGNAKAVDMLEQFLAEEHSIGAVELIAIDCVPEEMQPSARIKVYVHTMSNSFQTVRKYMTMGGRCMDPATLEGLENLHDVWYSLLGESQGIVNEEYSKPLTGFSSMQHHLYFSYEMTPGNADPGVKVYIPVQSYAPDDKTIAQNYEANFRQLNWPWGEPGVYEAVIESALGPVKHSRATFLHGGSSFIFSKGRGVYQSIYLDPPLEEGGNIAVFEHHDDQDTIVDLGNM

SEQ ID NO: 54 - DNA, 742 bp, Neotyphodium lolii
atggagcaagcgccgctcgcttatcaggaagttcaatggctagctgaaacttttgtcactttcatggggcttggctggcttatcaattacgtcttgatgatctggcactctaggaggggtgaaccgagcagcatggctctcatacccctctgcaacaacatcgcctgggagctcgtatacacgattatctatccgtctcctaacaaagtggaacttgcggctttcatagcaggtgtcactttgaacttccttatcatgacctctgcagcccgttcggcaagatccgagtggagtcactcacccacaatggctaagcatgcaggtttgattatagtcgcaggaatattgatgtgcttcaccggacatgtagcattggcgatggaaataggacctgcgcttgcttactcatggggagctgtcatatgccaactagctctaagcattggaggcgtgtgtcaattgttgcagcagcatagtactggtgggacatcatggaaactttggtaagtgaataaatcaattacgtttctaatctatattgaatgtcatatcaggggtggctgacatgaaagttttcaggtcaagtcgatttctaggctcttgttgtgcggttggctttgcctttcttcgctggagatactggcccgaggcgtacggatggctggccagtccccttatcctctggagtcttgccacgtttcttgtggccgatttgacgtacggggtttgtctccttctttag

SEQ ID NO: 55 - protein, 227 aa, Neotyphodium lolii
MDGFSNMEQAPLAYQEVQWLAETFVTFMGLGWLINYVLMIWHSRRGEPSSMALIPLCNNIAWELVYTIIYPSPNKVELAAFIAGVTLNFLIMTSAARSARSEWSHSPTMAKHAGLIIVAGILMCFTGHVALAMEIGPALAYSWGAVICQLALSIGGVCQLLQQHSTGGTSWKLWSSRFLGSCCAVGFAFLRWRYWPEAYGWLASPLILWSLATFLVADLTYGVCLLL